(19) United States
(12) Patent Application Publication
(10) Pub. No.: US 2026/0053514 A1
(43) Pub. Date: Feb. 26, 2026

(54) CATHETER SYSTEM FOR TREATING THROMBOEMBOLIC DISEASE HAVING DEEP PULSATILE SUCTION WITH DIFFERENTIAL FLOW AND METHOD OF USING SAME

(71) Applicant: Imperative Care, Inc., Campbell, CA (US)

(72) Inventors: Michael Buck, Menlo Park, CA (US); Julia Fox, San Carlos, CA (US); James Jacobs, Danville, CA (US)

(21) Appl. No.: 19/311,964

(22) Filed: Aug. 27, 2025

Related U.S. Application Data
(63) Continuation-in-part of application No. PCT/US2024/018161, filed on Mar. 1, 2024.
(60) Provisional application No. 63/557,391, filed on Feb. 23, 2024.

(51) Int. Cl. A61B 17/22; A61B 17/00; A61M 1/00; A61M 39/24

(52) U.S. Cl. CPC A61B 17/22; A61B 17/00234; A61M 1/75; A61M 39/24; A61B 2017/00238; A61B 2017/00561; A61B 2017/22079; A61B 2217/005; A61M 2202/0014; A61M 2202/06; A61M 2205/3334; A61M 2205/3344; A61M 2210/12

(57) ABSTRACT

A vacuum aspiration system and methods of use are disclosed. The system and method can be configured to provide deep pulsatile suction to a catheter body to more efficiently aspirate a clot material from a patient. In some embodiments, a deep pulse can include a rapid increase in flow rate of the fluid through the catheter body to a sustained high flow rate range for a short period of time followed by a rapid decrease in the flow rate to a low or negligible flow rate level to minimize blood loss without any changes to the suction pressure being applied to the catheter.

### CROSS-REFERENCE TO RELATED APPLICATIONS

This application is a continuation-in-part application of International Patent Application No. PCT/US2024/018161 filed Mar. 1, 2024, which claims the priority benefit under 35 U.S.C. § 119(e) of U.S. Provisional Patent Application No. 63/557,391, filed Feb. 23, 2024, the entireties of both of which are hereby incorporated by reference herein.

### BACKGROUND OF THE DISCLOSURE

Thrombotic restrictions and occlusions within a patient's blood vessels are a significant medical problem and often require intervention to remove these restrictions and blockages to restore health to patients. While applicable to a wide range of vascular applications in both the arterial and venous systems, including a variety of small vessels, the following background illuminates the problems primarily through the example of patients suffering with Pulmonary Embolism.

Venous thromboembolic disease (VTE) is a worldwide crisis. There are over 10 million cases of deep vein thrombosis (DVT) and pulmonary embolism (PE) diagnosed globally per year, with 1 million cases occurring in the United States and over 700,000 in France, Italy, Germany, Spain, Sweden, and the United Kingdom combined each year. There are approximately 60,000 to 100,000 deaths from PE in the United States each year. DVT and PE are part of the same continuum of disease, with over 95% of emboli originating in the lower extremities. When PE occurs, the severity depends on the embolic burden and its effect on the right ventricle as well as underlying cardiopulmonary comorbidities. Death can result from the acute increase in pulmonary artery (PA) pressure with increased right ventricular (RV) afterload and dysfunction.

Patients with high-risk pulmonary embolism (PE) are treated primarily with thrombolytic therapy delivered systemically or locally through Catheter Directed Thrombolytics. These approaches result in catheterization lab visits, lengthy hospital stays and often lead to bleeding complications. Newer approaches to PE treatment include single session thrombectomy treatments without the use of thrombolytics. These thrombectomy treatments include delivering a catheter into the PA to remove the thrombus or embolus (clot) through aspiration, and secondary tools may also macerate or disrupt the clot prior to or during aspiration. While thrombectomy results in fewer bleeding complications and reduced hospital stays compared to thrombolytics, there is much to be improved upon given the challenges of the procedure itself, including the ability to capture a broad spectrum of clot types and reduce the total volume of blood loss during the procedure.

The thrombectomy catheter is introduced through an introducer sheath in a large diameter vein. A flexible guide wire is passed through the introducer into the vein. The flexible guidewire provides a rail for a flexible guide catheter to be advanced through the right atrium into the right ventricle and into the pulmonary artery. The flexible guidewire is removed and replaced with a stiff guidewire. The large diameter thrombectomy catheter with support dilator is then advanced over the stiff guidewire to the pulmonary artery and the dilator is removed. If the large diameter thrombectomy catheter is not successful in accessing or aspirating thrombus in a more distal portion of the vessel, a smaller diameter catheter may be inserted through the large diameter catheter.

In addition, peripheral arterial occlusive (PAO) disease occurs in more than 4% of individuals over age 40 and markedly increases in incidence after the age of 70. Acute PAO is usually due to thrombosis of the peripheral vasculature and is associated with a significant risk of limb loss. In order to preserve the limb, therapy for acute PAO centers on the rapid restoration of arterial patency and blood flow such as through mechanical thrombectomy in procedures similar to those described above.

Clot aspiration using certain commercial vacuum-assisted thrombectomy systems may sometimes need to be terminated due to the risk of excessive blood loss by the patient, especially when using large aspiration catheters. During aspiration thrombectomy, when the catheter tip falls out of contact with the thrombus or other occlusive material, the tip is exposed to healthy blood and full flow of blood through the catheter ensues. Under such conditions, the total volume of blood loss is excessive, and in some cases, may result in premature termination of the procedure. For example, during a procedure when the catheter enters healthy blood and full aspiration flow ensues, the blood loss rate can be on the order of 30-40 cc per second with a 24 French size catheter. The catheter cannot run in unrestricted mode for more than approximately 10 to 15 seconds, as the aggregate blood loss may reach an unacceptable level before sufficient clot is removed.

### SUMMARY OF SOME EXAMPLE EMBODIMENTS

Disclosed herein are embodiments of a vacuum aspiration system. In some embodiments, the vacuum aspiration system can include an aspiration catheter comprising a proximal end and a distal end, a housing coupled with the proximal end of the aspiration catheter, a fluid flow path extending through the aspiration catheter and the housing, a source of suction, a suction conduit configured to be in communication with the source of suction and the fluid flow path extending through the aspiration catheter and the housing, and an aspiration control valve configured to control a suction pressure provided to the fluid flow path through the suction conduit from the source of suction. In some embodiments, the vacuum aspiration system can be configured such that, with the aspiration control valve in a closed state and the source of suction providing suction to the fluid flow path to the aspiration control valve, when the aspiration control valve is moved to an open state, the source of suction will provide a rapid burst of suction through the aspiration catheter. A peak flow rate of a fluid through the fluid flow path in some embodiments is achieved within 0.08 seconds or approximately 0.08 seconds after suction is provided to the fluid flow path. In some embodiments, the rapid burst of suction is followed by reduced flow.

In some embodiments, a clot container may be positioned in the fluid flow path, with the suction conduit positioned downstream of the clot container. In some embodiments, the suction conduit has a smaller diameter than a diameter of the fluid flow path extending through the aspiration catheter and the housing to limit the flow rate of the fluid through the fluid flow path. In some embodiments, the vacuum aspiration system can have a conduit or tube that fluidically couples the suction conduit to a collection canister and/or aspiration pump or source of suction. In some embodiments, the diameter of such conduit or tube can also limit the flow rate of the fluid through the fluid flow path, e.g., downstream of the clot container. In some embodiments, the clot container may be positioned downstream of the aspiration control valve so that suction from the source of suction is always applied to the clot container when the source of suction is on or operating, regardless of whether the aspiration control valve is in an open state or a closed state. In some embodiments, the canister can be configured to have an air passageway in the canister configured to allow outside air to be drawn into the canister. The air passageway can be, in some embodiments, configured to restrict or prevent air from flowing or leaking out of the canister from inside the canister, or can have a filter configured to restrict or prevent bacteria, pathogens, or other harmful contaminants from leaving the container. In some embodiments, the air passageway can be controllable so that a flow rate of air entering the canister through the air hole can be regulated or controlled, e.g., adjusted or even terminated, as desired.

Also disclosed herein are embodiments of a vacuum aspiration system having an aspiration catheter assembly that can include a catheter sheath extending from a proximal end to a distal end, a housing at the proximal end of the catheter sheath, a fluid flow path extending through the housing and the catheter sheath and configured to selectively receive a suction pressure from a source of suction, and an aspiration control valve in fluid communication with the fluid flow path, the aspiration control valve configured to control the suction pressure through the fluid flow path from the source of suction upstream of the aspiration control valve.

In some embodiments, the vacuum aspiration system can be configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that: (i) a flow rate of a fluid through the fluid flow path upstream of the aspiration control valve increases to a first flow rate range that is greater than zero; (ii) while the aspiration control valve continues to be in the open state, the flow rate of the fluid through the fluid flow path is maintained at the first flow rate range for a first period of time; and (iii) while the aspiration control valve continues to be in the open state, without any substantial changes (e.g., any changes) to the suction pressure provided by the source of suction, after the first period of time, the flow rate of the fluid through the fluid flow path drops to a second flow rate range that is greater than zero and that is less than the first flow rate range. In some embodiments, a total volume of the fluid aspirated through the catheter sheath is less than 60 ml when the flow rate of the fluid through the fluid flow path drops to the second flow rate range.

Also disclosed herein are embodiments of a vacuum aspiration system that can include an aspiration catheter assembly that can include a catheter sheath extending from a proximal end to a distal end, a housing at the proximal end of the catheter sheath, a fluid flow path extending through the housing and the catheter sheath and configured to selectively receive a suction pressure from a source of suction, and an aspiration control valve in fluid communication with the fluid flow path, the aspiration control valve configured to control the suction pressure through the fluid flow path from the source of suction upstream of the aspiration control valve.

In some embodiments, the vacuum aspiration system can be configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that: (i) a flow rate of a fluid through the fluid flow path increases to a first flow rate range that is greater than zero; (ii) while the aspiration control valve continues to be in the open state, the flow rate of the fluid through the fluid flow path is maintained at the first flow rate range until a first volume of the fluid has been aspirated through the catheter sheath; and (iii) while the aspiration control valve continues to be in the open state, after the first volume of the fluid has been aspirated through the catheter sheath, the flow rate of the fluid through the fluid flow path drops to a second flow rate range that is greater than zero but less than the first flow rate range.

Also disclosed herein are embodiments of a vacuum aspiration system having an aspiration catheter assembly that can include a catheter sheath extending from a proximal end to a distal end, a housing at the proximal end of the catheter sheath, a fluid flow path extending through the housing and the catheter sheath and configured to selectively receive a suction pressure from a source of suction, and an aspiration control valve in fluid communication with the fluid flow path, the aspiration control valve configured to control the suction pressure through the fluid flow path from the source of suction upstream of the aspiration control valve, wherein the vacuum aspiration system can be configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that a peak flow rate of a fluid through the fluid flow path is achieved within 0.08 seconds (or approximately 0.08 seconds) after suction is provided to the fluid flow path.

Additionally, any embodiments of the devices, systems, and methods disclosed herein can include, in additional embodiments, one or more of the following features, components, and/or details, in any combination with any of the other features, components, and/or details of any other embodiments disclosed herein: wherein the first flow rate range is greater than 100 ml per second; wherein the first flow rate range is greater than 150 ml per second; wherein the first flow rate range is greater than 180 ml per second; wherein the first flow rate range is greater than 190 ml per second; wherein the total volume of the fluid aspirated through the catheter sheath is less than 55 ml when the flow rate of the fluid through the fluid flow path drops to the second flow rate range; wherein the total volume of the fluid aspirated through the catheter sheath is less than 50 ml when the flow rate of the fluid through the fluid flow path drops to the second flow rate range; wherein the first period of time is from 0.1 seconds or approximately 0.1 seconds to 0.3 seconds or approximately 0.3 seconds (i.e., greater than or equal to 0.1 seconds or approximately 0.1 seconds and less than or equal to 0.3 seconds or approximately 0.3 seconds); wherein the first period of time is from 0.1 seconds or approximately 0.1 seconds to 0.25 seconds or approximately 0.25 seconds (i.e., greater than or equal to 0.1 seconds or approximately 0.1 seconds and less than 0.25 seconds or approximately 0.25 seconds); wherein the first period of time is from 0.1 seconds or approximately 0.1 seconds to 0.2 seconds or approximately 0.2 seconds (i.e., greater than or equal to 0.1 seconds or approximately 0.1 seconds and less than 0.2 seconds or approximately 0.2 seconds); wherein the second flow rate range is less than half of the first flow rate range; wherein the second flow rate range is less than 25% of the first flow rate range; wherein the second flow rate range is less than 15% of the first flow rate range; wherein the second flow rate range is less than 10% of the first flow rate range; wherein the second flow rate range is less than 5% of the first flow rate range; wherein the second flow rate range is less than 30 ml per second; wherein the second flow rate range is less than 20 ml per second; wherein the second flow rate range is less than 10 ml per second; and/or wherein the first volume of the fluid is 16 ml or approximately 16 ml, 17.5 ml or approximately 17.5 ml, 18 ml or approximately 18 ml, 19 ml or approximately 19 ml, 20 ml or approximately 20 ml, or from 10 ml or approximately 10 ml to 30 ml or approximately 30 ml, or from 15 ml or approximately 15 ml to 25 ml or approximately 25 ml, or of any value, approximate value, or range of values in any of the foregoing ranges.

Additionally, any embodiments of the devices, systems, and methods disclosed herein can include, in additional embodiments, one or more of the following features, components, and/or details, in any combination with any of the other features, components, and/or details of any other embodiments disclosed herein: wherein the source of suction applies a suction pressure of −28 inHg or approximately −28 inHg, or from −26 inHg or approximately −26 inHg to −29.92 inHg or approximately −29.92 inHg; wherein the source of suction is a vacuum pump; wherein the source of suction is a syringe, wherein the aspiration catheter further includes a suction conduit in communication with the fluid flow path and configured to be fluidically coupled with the source of suction; wherein the aspiration control valve is moved from the closed state to the open state rapidly enough to not affect the flow rate of the fluid through the flow path; wherein the vacuum aspiration system can be configured to provide a rapid burst of suction through the catheter so that the catheter reaches a peak flow rate of a fluid through the fluid flow path within 0.06 seconds; wherein the fluid is water; wherein the vacuum aspiration system can be configured to provide a rapid burst of suction through the catheter so that the catheter reaches a peak flow rate of a fluid through the fluid flow path at approximately 0.055 seconds; wherein the fluid is water; wherein the vacuum aspiration system can be configured to provide a rapid burst of suction through the catheter so that the catheter reaches a peak flow rate of a fluid through the fluid flow path within 0.03 seconds to 0.06 seconds; wherein the fluid is water; wherein the fluid is water and the fluid flow path catheter is primed with the fluid before suction is applied to the catheter; wherein the fluid is water, and the peak flow rate of the water through the fluid flow path is 183 ml per second; wherein the fluid is water, and the peak flow rate of the water through the fluid flow path is from 173 ml per second to 193 ml per second; and/or wherein the fluid is water, and the peak flow rate of the water through the fluid flow path is greater than 180 ml per second.

In some embodiments, wherein the fluid is water, the average flow rate of the water through the fluid flow path of a 24 Fr catheter during the initial pulse (i.e., from the point when the flow rate increases due to the aspiration control valve being opened through the point where the flow rate has reached its lowest relative value after the initial peak flow rate or that, in some embodiments, corresponds to the clot container being full) is 128 ml per second or approximately 128 ml per second, or is from 100 ml per second or approximately 100 ml per second to 150 ml per second or approximately 150 ml per second, or is from 110 ml per second or approximately 110 ml per second to 140 ml per second or approximately 140 ml per second, or is from 120 ml per second or approximately 120 ml per second to 135 ml per second or approximately 135 ml per second, or is of any value, approximate value, or range of values of any of the foregoing ranges.

In some embodiments, wherein the fluid is a blood analog, the average flow rate of the fluid through the fluid flow path of a 24 Fr catheter during the initial pulse (i.e., from the point when the flow rate increases due to the aspiration control valve being opened through the point where the flow rate has reached its lowest relative value after the initial peak flow rate or that, in some embodiments, corresponds to the clot container being full) is 104 ml per second or approximately 104 ml per second, or is from 80 ml per second or approximately 80 ml per second to 120 ml per second or approximately 120 ml per second, or is from 90 ml per second or approximately 90 ml per second to 115 ml per second or approximately 115 ml per second, or is from 100 ml per second or approximately 100 ml per second to 110 ml per second or approximately 110 ml per second, or is of any value, approximate value, or range of values of any of the foregoing ranges. In some embodiments, the blood analog can consist of 49.96% glycerin or approximately 49.96% glycerin, 49.96% water or approximately 49.96% water, and 0.075% xantham gum or approximately 0.075% xantham gum.

In some embodiments, the vacuum aspiration system can be configured so that the peak flow rate of the fluid through the catheter is sustained for no more than 0.3 seconds, or so that the peak flow rate of the fluid through the catheter is sustained for no more than 0.2 seconds, or so that the peak flow rate of the fluid through the catheter is sustained for no more than 0.15 seconds.

Additionally, any embodiments of the devices, systems, and methods disclosed herein can include, in additional embodiments, one or more of the following features, components, and/or details, in any combination with any of the other features, components, and/or details of any other embodiments disclosed herein: wherein the vacuum aspiration system can be configured to provide a rapid drop-off of the suction provided through the catheter such that the flow rate of the fluid through the fluid flow path drops below 20 mL per second within 0.4 seconds after the flow rate of the fluid through the fluid flow path first reaches the peak flow rate while the aspiration control valve remains in an open state; wherein the vacuum aspiration system can be configured to provide a rapid drop-off of the suction provided through the catheter such that the flow rate of the fluid through the fluid flow path drops below 10 mL per second within 0.3 seconds after the flow rate of the fluid through the fluid flow path first reaches the peak flow rate while the aspiration control valve remains in an open state; wherein the vacuum aspiration system can be configured to provide a rapid drop-off of the suction provided through the catheter such that the flow rate of the fluid through the fluid flow path drops below 10 mL per second within 0.2 seconds after the flow rate of the fluid through the fluid flow path first reaches the peak flow rate while the aspiration control valve remains in an open state; wherein the aspiration control valve can be configured to move between a first position wherein the aspiration control valve is closed and a second position wherein the aspiration control valve is open, and wherein suction is provided to the fluid flow path by moving the aspiration control valve to the second position, thereby providing the suction from the source of suction to the fluid flow path; and/or wherein the aspiration control valve comprises a lever;
Additionally, any embodiments of the devices, systems, and methods disclosed herein can include, in additional embodiments, one or more of the following features, components, and/or details, in any combination with any of the other features, components, and/or details of any other embodiments disclosed herein: wherein the aspiration catheter further includes a clot container coupled with the housing, the clot container being in fluid communication with the fluid flow path; wherein the clot container has a filter therein; wherein the fluid flow path passes through a suction conduit that fluidically couples to the source of suction; wherein the suction conduit has an internal diameter (D8, e.g., as illustrated in FIG. 28A) of 2.67 mm, or approximately 2.67 mm, or from 2 mm or approximately 2 mm to 4 mm or approximately 4 mm, or from 2.5 mm or approximately 2.5 mm to 3 mm or approximately 3 mm, or of any value, approximate value, or range of values in any of the foregoing ranges; wherein the aspiration catheter further includes a clot container coupled with the housing, the clot container having an internal space, an inlet, and an outlet; wherein the outlet has a conduit that is in fluid communication with the fluid flow path; wherein the conduit of the outlet of the clot container has a minimum internal diameter (D6, e.g., as illustrated in FIGS. 28A and 28B) of 2 mm, or approximately 2 mm, or from 1 mm or approximately 1 mm to 3 mm or approximately 3 mm, or from 1.5 mm or approximately 1.5 mm to 2.5 mm or approximately 2.5 mm, or of any value, approximate value, or range of values in any of the foregoing ranges; wherein the conduit of the outlet of the clot container has a first internal diameter (D6) of 2 mm, or approximately 2 mm, or from 1 mm or approximately 1 mm to 3 mm or approximately 3 mm, or from 1.5 mm or approximately 1.5 mm to 2.5 mm or approximately 2.5 mm, or of any value, approximate value, or range of values in any of the foregoing ranges, and a second internal diameter (D7, e.g., as illustrated in FIGS. 28A and 28B) of 2.25 mm, or approximately 2.25 mm, or from 1.5 mm or approximately 1.5 mm to 3 mm or approximately 3 mm, or from 2 mm or approximately 2 mm to 2.5 mm or approximately 2.5 mm, or of any value, approximate value, or range of values in any of the foregoing ranges; wherein the outlet is upstream of the inlet; comprising a conduit extending from the aspiration control valve to the inlet of the clot container having an internal diameter (D5, e.g., as illustrated in FIG. 28A) of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges; comprising a conduit extending from the aspiration control valve upstream of the aspiration control valve toward a proximal end of the catheter sheath, the conduit extending from the aspiration control valve having an internal diameter (D3, D4, e.g., as illustrated in FIG. 28A) through the conduit of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges; comprising a sealing element at a proximal end of the catheter sheath, the sealing element having a passageway therethrough that is part of the fluid flow path and that has a minimum internal diameter (D1, e.g., as illustrated in FIG. 28A) of 4.67 mm, or approximately 4.67 mm, or from 3.7 mm or approximately 3.7 mm to 5.7 mm or approximately 5.7 mm, or from 4.2 mm or approximately 4.2 mm to 5.2 mm or approximately 5.2 mm, or of any value, approximate value, or range of values in any of the foregoing ranges; wherein the sealing element has a first end that is distal to a second end, the first end being upstream of the first end in the fluid pathway; wherein the minimum internal diameter (D1) is at the first end and the second end has an internal diameter (D2, e.g., as illustrated in FIG. 28A) of 5.14 mm, or approximately 5.14 mm, or from 4 mm or approximately 4 mm to 6.5 mm or approximately 6.5 mm, or from 4.5 mm or approximately 4.5 mm to 6 mm or approximately 6 mm, or of any value, approximate value, or range of values in any of the foregoing ranges; wherein the aspiration catheter further includes a clot container coupled with the housing, an inlet into the clot container having an internal diameter of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges, and a an outlet from the clot container downstream of the inlet into the clot container, the outlet having an internal diameter of 2.8 mm, or approximately 2.8 mm, or from 1.8 mm or approximately 1.8 mm to 3.8 mm or approximately 3.8 mm, or from 2.5 mm or approximately 2.5 mm to 3.1 mm or approximately 3.1 mm, or of any value, approximate value, or range of values in any of the foregoing ranges; and/or wherein the flow rate drops from the first flow rate to the second flow rate in 0.05 seconds, approximately 0.05 seconds, or less than 0.05 seconds, or in 0.075 seconds, approximately 0.075 seconds, or less than 0.075 seconds, or from 0.025 seconds or approximately 0.025 seconds to 0.075 seconds or approximately 0.075 seconds, or from 0.04 seconds or approximately 0.04 seconds to 0.06 seconds or approximately 0.06 seconds, or of any value, approximate value, or range of values in any of the foregoing ranges. In some embodiments, the tubing or conduit downstream of the clot container can be larger and the device can have a constrictor that can be used to reduce the flow rate downstream of the clot container. In some embodiments, the constrictor can be adjustable to adjust the flow rate downstream of the clot container.

Additionally, any embodiments of the devices, systems, and methods disclosed herein can include, in additional embodiments, one or more of the following features, components, and/or details, in any combination with any of the other features, components, and/or details of any other embodiments disclosed herein: wherein the aspiration catheter further includes a clot container coupled with the housing; wherein the flow rate drops from the first flow rate to the second flow rate when the clot container becomes full with the fluid that has been aspirated through the catheter sheath; wherein the clot container has a volume of 17.7 ml or approximately 17.7 ml, or from 15 ml or approximately 15 ml to 20 ml or approximately 20 ml, or from 17 ml or approximately 17 ml to 19 ml or approximately 19 ml, or of any value, approximate value, or range of values in any of the foregoing ranges; wherein the clot container has a volume of 40 ml or approximately 40 ml, or a volume of 60 ml or approximately 60 ml, or from 30 ml or approximately 30 ml to 80 ml or approximately 80 ml, or from 40 ml or approximately 40 ml to 70 ml or approximately 70 ml, or of any value, approximate value, or range of values in any of the foregoing ranges; wherein the first period of time is increased by increasing the volume of the clot container and/or a volume of the fluid flow path upstream of the clot container; wherein dropping the flow rate of fluid through the fluid flow path to the second flow rate after the first period of time reduces (e.g., substantially reduces) a loss of blood from a patient during a thrombectomy procedure; wherein the aspiration catheter assembly is a 24 Fr aspiration catheter; wherein the aspiration catheter assembly is a 16 Fr aspiration catheter; wherein the fluid is at room temperature; wherein the fluid is at a temperature between 68 degrees F. and 73 degrees F.; wherein the fluid is homogeneous (e.g., does not have any clots, thicker, or more viscous substances therein); wherein a volume of the fluid flow path downstream of the catheter sheath and downstream to and including the clot container is 22.6 ml or approximately 22.6 ml, or from 18 ml or approximately 18 ml to 25 ml or approximately 25 ml, or from 20 ml or approximately 20 ml to 25 ml or approximately 25 ml, or of any value, approximate value, or range of values in any of the foregoing ranges, wherein the aspiration catheter further includes a suction conduit in communication with the fluid flow path and configured to be fluidically coupled with the source of suction; wherein a volume of the fluid pathway from and including the suction conduit upstream up to the clot container is 1.8 ml or approximately 1.8 ml, or from 1.5 ml or approximately 1.5 ml to 2.0 ml or approximately 2.0 ml, or from 1.7 ml or approximately 1.7 ml to 1.9 ml or approximately 1.9 ml, or of any value, approximate value, or range of values in any of the foregoing ranges.

Also disclosed herein are embodiments of a method of aspirating a fluid through a catheter of an aspiration system, that include positioning an aspiration control valve of the catheter in a closed position, with the aspiration control valve in the closed position and a fluid flow path of the catheter in fluid communication with the fluid, applying a suction pressure to a fluid flow path of the catheter, moving the aspiration control valve to an open position to aspirate the fluid through the fluid flow path of the catheter; wherein a flow rate of the fluid through the fluid flow path increases to a first flow rate range that is greater than 50 ml per second, and when the flow rate of the fluid through the fluid flow path decreases below a second value, moving the aspiration control valve back to the closed position. In some embodiments, the catheter can be configured to decrease the flow rate of the fluid through the fluid flow path without changing the suction pressure being applied to the fluid flow path of the catheter.

Additionally, any embodiments of the devices, systems, and methods disclosed herein can include, in additional embodiments, one or more of the following features, components, details, and/or steps, in any combination with any of the other features, components, details, and/or steps of any other embodiments disclosed herein: wherein the catheter can be configured to decrease the flow rate of the fluid through the fluid flow path to the second value of the flow rate range without making any changes to the aspiration system; wherein the first flow rate range is greater than 100 ml per second or approximately 100 ml per second, or is greater than 150 ml per second or approximately 150 ml per second, or is greater than 180 ml per second or approximately 180 ml per second, or is greater than 190 ml per second or approximately 190 ml per second, or is greater than 200 ml per second or approximately 200 ml per second, or is from 100 ml per second or is approximately 100 ml per second to 200 ml per second or approximately 200 ml per second, or is from 150 ml per second or approximately 150 ml per second to 200 ml per second or approximately 200 ml per second, or of any value, approximate value, or range of values in any of the foregoing ranges; wherein the second value is between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges.

Also disclosed herein are embodiments of a method of aspirating a clot material during a thrombectomy procedure. In any embodiments disclosed herein, the method, which can be referred to as the DP2 or deep pulse withdrawal method, can include positioning an aspiration control valve of the aspiration catheter in a closed position, applying a suction pressure to a fluid flow path of the catheter, positioning an aspiration catheter within a predetermined distance of a clot within a patient's vasculature, and moving the aspiration control valve to an open position to aspirate the clot through the fluid flow path of the aspiration catheter wherein a flow rate of a fluid through the fluid flow path upstream of the aspiration control valve increases to a first flow rate range that is greater than zero. Thereafter, when the flow rate of the fluid through the fluid flow path decreases below a second flow rate value, the user can thereafter move the aspiration control valve back to the closed position, withdraw the aspiration catheter a predetermined distance and, after withdrawing the aspiration catheter the predetermined distance, move the aspiration control valve again to the open position to continue to aspirate the clot through the fluid flow path of the aspiration catheter, wherein the flow rate of the fluid through the fluid flow path upstream of the aspiration control valve increases again to the first flow rate range.

In some embodiments, the method can further include, when the flow rate of the fluid through the fluid flow path again decreases below the second flow rate value, moving the aspiration control valve back to the closed position. In some embodiments, the method can further include withdrawing the aspiration catheter by a second predetermined distance and, after withdrawing the aspiration catheter the second predetermined distance, moving the aspiration control valve again to the open position to continue to aspirate the clot through the fluid flow path of the aspiration catheter, wherein the flow rate of the fluid through the fluid flow path upstream of the aspiration control valve increases again to the first flow rate range. In some embodiments, the method can further include, when the flow rate of the fluid through the fluid flow path decreases below the second flow rate value for a third time, moving the aspiration control valve back to the closed position.

In some embodiments, the method can further include withdrawing the aspiration catheter by a third predetermined distance and, after withdrawing the aspiration catheter the third predetermined distance, moving the aspiration control valve again to the open position to continue to aspirate the clot through the fluid flow path of the aspiration catheter, wherein the flow rate of the fluid through the fluid flow path upstream of the aspiration control valve increases again to the first flow rate range. In some embodiments, the method can further include, when the flow rate of the fluid through the fluid flow path decreases below the second flow rate value for a third time, moving the aspiration control valve back to the closed position. In some embodiments, this method can be repeated again as many times as desired or needed for a more complete aspiration of the clot material.

In other embodiments, the user can distally advance the aspiration catheter a predetermined distance, or a second predetermined distance, or a third predetermined distance, or otherwise (instead of withdrawing the aspiration catheter a predetermined distance or a second predetermined distance, or a third predetermined distance, or otherwise) and, after advancing the aspiration catheter the predetermined distance, or a second predetermined distance, or a third predetermined distance, or otherwise, move the aspiration control valve again to the open position to continue to aspirate the clot through the fluid flow path of the aspiration catheter, wherein the flow rate of the fluid through the fluid flow path upstream of the aspiration control valve increases again to the first flow rate range.

In any embodiments disclosed herein, the predetermined distance (which can include the second predetermined distance and/or the third predetermined distance) can be 0 cm or approximately 0 cm, 2 cm or approximately 2 cm, 4 cm or approximately 4 cm, 5 cm, approximately 5 cm, or more than 5 cm, or from 0 cm or approximately 0 cm to 5 cm, approximately 5 cm, or more than 5 cm, or from 1 cm or approximately 1 cm to 4 cm or approximately 4 cm, or of any of the values, approximate values or range of values of any of the foregoing ranges. In any embodiments disclosed herein, the predetermined distance, the second predetermined distance, and the third predetermined distance can each be the same or can each be different.

Additionally, any embodiments of the devices, systems, and methods disclosed herein can include, in additional embodiments, one or more of the following features, components, details, and/or steps, in any combination with any of the other features, components, details, and/or steps of any other embodiments disclosed herein: wherein the first flow rate range is greater than 40 ml per second; wherein the first flow rate range is greater than 50 ml per second; wherein the first flow rate range is greater than 60 ml per second., when the fluid is blood; wherein the first flow rate range is from 40 ml per second to 120 ml per second when the fluid is blood; wherein the first flow rate range is from 50 ml per second to 100 ml per second when the fluid is blood; wherein the second flow rate value is between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges; wherein the fluid is blood; wherein the flow rate of the fluid through the fluid flow path decreases below the second flow rate value when a clot container of the aspiration catheter becomes full with the fluid or the fluid and the clot material; and/or wherein the aspiration catheter can be configured to decrease the flow rate of the fluid through the fluid flow path below the second flow rate value without any user input.

Also disclosed herein are embodiments of a vacuum aspiration system having an aspiration catheter assembly that can include a catheter having a proximal end and a distal end, a housing at the proximal end of the catheter, a fluid flow path extending through the housing and the catheter and configured to selectively receive a suction pressure from a source of suction, and an aspiration control valve in fluid communication with the fluid flow path. The aspiration control valve can be configured to control the suction pressure through the fluid flow path from the source of suction upstream of the aspiration control valve.

In some embodiments, the fluid flow path can include a conduit (also referred to herein as an aspiration conduit) that is in fluid communication with the catheter. The conduit can merge or connect with the catheter at or near a proximal end of the catheter. For example and without limitation, the conduit can have an end portion that merges and connects with the catheter at or near a proximal end of the catheter. The end portion of the catheter can be designed and configured to provide a smooth and gradual transition of fluid and/or material flowing through the catheter into the conduit. For example and without limitation, the end portion of the conduit can be curved and can have an end that is tangent to the catheter, so as to provide a smooth transition away from the catheter, wherein the portion of the conduit adjacent to the end curves away from the catheter tube. In some embodiments, the conduit can be in fluid communication with the aspiration control valve.

Embodiments of the vacuum aspiration system disclosed herein can have any of the components, features, or other details of any of the other vacuum aspiration system embodiments disclosed herein. In some embodiments, the vacuum aspiration system can be configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve (e.g., through the conduit and the catheter) such that a peak flow rate of a fluid through the fluid flow path is achieved rapidly after suction is provided to the fluid flow path.

Also disclosed herein are embodiments of an aspiration catheter assembly. In some embodiments, the aspiration catheter assembly is configured such that an aspiration conduit extending or branching away from the catheter sheath branches away from the catheter sheath at a very small angle relative to the catheter sheath. In some embodiments, the aspiration conduit branches away from the catheter sheath at a tangent to the catheter sheath. In any embodiments, the aspiration catheter assembly can include a catheter sheath having a conduit extending therethrough, a housing at a proximal end portion of the catheter sheath, an aspiration conduit within the handle, the aspiration conduit being in fluid communication with the conduit extending through the catheter sheath, and a fluid flow path extending through the catheter sheath and the aspiration conduit, the fluid flow path being configured to selectively receive a suction pressure from a source of suction. In some embodiments, the aspiration conduit branches away from the conduit extending through the catheter sheath and a junction with the catheter sheath. In some embodiments, the aspiration conduit has an end portion that is curved and is configured such that an initial portion of the fluid flow path going through the aspiration conduit from the conduit extending through the catheter sheath is tangent to the conduit extending through the catheter sheath at the junction between the catheter sheath and the aspiration conduit.

In some embodiments, the aspiration catheter assembly can include an aspiration control valve in fluid communication with the fluid flow path, the aspiration control valve configured to control the suction pressure through the fluid flow path from a source of suction upstream of the aspiration control valve. In some embodiments, the aspiration catheter assembly can include a clot container in fluid communication with the fluid flow path. The aspiration catheter assembly can include a constrictor in the fluid flow path downstream of the clot container, the constrictor configured to reduce the flow rate of the fluid through the fluid flow path downstream of the clot container. In some embodiments, the aspiration catheter assembly can include a clot container coupled with the housing, the clot container having an internal space, an inlet, and an outlet that is downstream of the inlet, wherein the outlet has a conduit that is in fluid communication with the fluid flow path, and wherein the conduit of the outlet of the clot container has a minimum internal diameter that is less than one-third of an internal diameter of conduit upstream of the clot container.

Disclosed herein are embodiments of a method of aspirating a substance with an aspiration device during an aspiration procedure. In some embodiments, the method can include, with an aspiration control valve of the aspiration device in a closed position, applying a suction pressure to a fluid flow path of the aspiration device, positioning a catheter of the aspiration device within a patient's vasculature, and moving the aspiration control valve to an open position to aspirate the substance through at least the catheter of the aspiration device so that a flow rate of the substance through the catheter increases to at least a first flow rate range that is greater than zero. In some embodiments, the first flow rate range can be greater than 60 ml per second. In some embodiments, the lowermost value of the first flow rate range can be greater than 120 ml per second.

The method can also include, when the flow rate of the substance through the catheter decreases below a second flow rate value that is lower than the first flow rate range, moving the aspiration control valve to the closed position and then moving the catheter in a proximal direction or in a distal direction. In some embodiments, the second flow rate range can be greater than zero and is less than 50% of the first flow rate range. After moving the catheter in the proximal direction or the distal direction, moving the aspiration control valve again to the open position to continue to aspirate the substance through at least the catheter. In some embodiments, moving the aspiration control valve again to the open position to continue to aspirate the substance through at least the catheter includes aspirating the substance through at least the catheter at a rate that is at least as great as the first flow rate range. In some embodiments, moving the aspiration catheter in the proximal direction or the distal direction includes moving the aspiration catheter in the proximal direction or the distal direction by a distance that is 2 cm or approximately 2 cm. In some embodiments, moving the aspiration catheter in the proximal direction or the distal direction includes moving the aspiration catheter in the proximal direction or the distal direction by a distance that is from 1 cm or approximately 1 cm to 3 cm or approximately 3 cm. In some embodiments, moving the aspiration catheter in the proximal direction or the distal direction includes moving the aspiration catheter in the proximal direction or the distal direction by a distance that is no greater than 3 cm.

In some embodiments, the method can further include when the flow rate of the substance through the fluid flow path again decreases below the second flow rate value, moving the aspiration control valve back to the closed position. In some embodiments, the method includes, after moving the aspiration control valve back to the closed position, moving the aspiration catheter again in the proximal direction or the distal direction and, after again moving the aspiration catheter in the proximal direction or the distal direction, again moving the aspiration control valve to the open position to continue to aspirate the substance through the fluid flow path of the aspiration catheter. Again moving the aspiration catheter in the proximal direction or the distal direction can include again moving the aspiration catheter by a distance that is 2 cm or approximately 2 cm. In some embodiments, wherein again moving the aspiration catheter in the proximal direction or the distal direction includes again moving the aspiration catheter by a distance that is from 1 cm or approximately 1 cm to 3 cm or approximately 3 cm. The method can further include, when the flow rate of the substance through the fluid flow path again decreases below the second flow rate value, moving the aspiration control valve back to the closed position. In some embodiments, the aspiration catheter can be configured to decrease the flow rate of the substance through the fluid flow path below the second flow rate value while the aspiration control valve is in the open position without any user input or any change to the suction pressure provided to the aspiration device by a source of suction.

Some embodiments disclosed herein are directed to a method of aspirating a substance from a patient's vasculature during an aspiration procedure, including providing an aspiration system comprising a catheter in communication with an aspiration pump through an aspiration line, a chamber in the aspiration line closer to the catheter than the pump, and a valve in the aspiration line between the catheter and the chamber, activating the aspiration pump to apply a suction force to the chamber, positioning the catheter within aspiration range of a clot, opening the valve a first time to aspirate the substance toward the chamber, closing the valve, moving the catheter in a distal or a proximal direction, and opening the valve a second time to aspirate the substance toward the chamber. In some embodiments, the method can include opening a vent to evacuate the chamber after closing the valve. In some embodiments, when the valve is an open state, the aspiration pump can aspirate the substance through at least the chamber to fill the chamber. In some embodiments, moving the catheter includes moving the catheter a distance that is from 1 to 3 cm.

### BRIEF DESCRIPTION OF THE DRAWINGS

FIG. 1 is a schematic view of a fluid management system in accordance with one aspect.

FIG. 2 is a schematic view as in FIG. 1, with a clot attached to a grasping catheter which extends through a large diameter catheter.

FIG. 3 is a schematic view as in FIG. 2, with the clot drawn into a transparent viewing tube on the large diameter access catheter.

FIG. 4 is a schematic view as in FIG. 3, with the clot advancing towards a thrombus collection chamber.

FIG. 5 is a schematic view as in FIG. 4, with the clot deposited in a transparent thrombus collection chamber.

FIG. 6 is a schematic view of a thrombectomy system configured to re-infuse filtered aspirated blood back into a patient.

FIG. 7A is a schematic view of an alternate configuration of the fluid management system.

FIG. 7B is a schematic view of an alternate configuration of the fluid management system.

FIG. 8 is a schematic view of a grasping catheter configured to apply suction to a clot.

FIG. 9 is a schematic view of an alternative aspiration system in accordance with another aspect, having a first thrombectomy catheter and a second thrombectomy catheter extending therethrough.

FIG. 10A is a schematic view of the hand piece for the first thrombectomy catheter of FIG. 9.

FIGS. 10B-10E illustrate interface details between a filter assembly and a handpiece.

FIG. 11A is a schematic view of the handpiece for the second thrombectomy catheter of FIG. 9.

FIG. 11B is a simplified flow diagram of the dual vacuum chamber aspiration system.

FIG. 11C is a qualitative fluid flow rate diagram at the catheter tip, following opening of the momentary vacuum control valve.

FIG. 12 is a schematic flow diagram for a three-way valve.

FIGS. 13A-13C illustrate three flow configurations for a three-way valve.

FIGS. 14A-14C illustrate operation of a hemostasis valve.

FIG. 14D illustrates an alternative filament configuration of the hemostasis valve.

FIGS. 15A-15B are schematic layouts of the components of a proximal handle of an aspiration catheter.

FIGS. 16A and 16B are different implementations of thrombus engagement tools.

FIG. 17A is a side elevational view of one thrombus engagement tool tip.

FIG. 17B is a longitudinal cross-section through the tip of FIG. 17A.

FIG. 18A is a side elevational view of an alternative thrombus engagement tip.

FIG. 18B is a longitudinal cross-section through the tip of FIG. 18A.

FIG. 19A is a side elevational view of a catheter and split dilator system in accordance with another aspect.

FIG. 19B shows the system of FIG. 19A, with the dilator partially retracted and peeled away from the guide wire with the guide wire progressively escaping from the dilator through an axially extending split.

FIG. 19C shows the dilator fully retracted from the catheter but still over the guide wire.

FIG. 19D shows the dilator fully removed from the catheter and the guide wire, leaving the catheter and guide wire unmoved from their position within the vasculature.

FIGS. 20A-20C show a proximal handle for a dilator.

FIG. 21 is a side elevational partial cross section of a catheter having a cannulated guide rail extending therethrough over a guidewire.

FIG. 22 is a cross sectional view through a dual dilator system such as that shown in FIG. 23.

FIG. 23 is a side elevational cross section of a distal portion of a dual dilator system in accordance with another aspect.

FIG. 24 is a cross section as in FIG. 23, with a distal tip formed by the tubular dilator.

FIG. 25 is a side elevational view of a portion of a tubular dilator having a separation line to allow longitudinal splitting of the sidewall during proximal retraction.

FIG. 26 is a schematic view of a thrombectomy catheter in accordance with another aspect.

FIG. 27A is a cross section view of the handle shown in FIG. 26 with the valve in a first position.

FIG. 27B is a cross section view of the handle shown in FIG. 26 with the valve in a second position.

FIG. 27C shows an embodiment of an aspiration catheter system having a first aspiration catheter and a second aspiration catheter.

FIG. 28A shows a cross section view of the handle shown in FIG. 26, showing inner diameter dimension characters D1-D8.

FIG. 28B shows a cross-sectional view of an embodiment of a clot container.

FIG. 29A shows the flow rate in ml per second over time for Experiment 1.

FIG. 29B shows the total volume aspirated in ml over time (in seconds) for Experiment 1.

FIGS. 29C and 29D show the data used to generate the plots of FIGS. 29A and 29B, respectively.

FIG. 30A shows the flow rate in ml per second over time for Experiment 2.

FIG. 30B shows the total volume aspirated in ml over time (in seconds) for Experiment 2.

FIGS. 30C and 30D show the data used to generate the plots of FIGS. 30A and 30B, respectively.

FIG. 31A shows the flow rate in ml per second over time for Experiment 3.

FIG. 31B shows the total volume aspirated in ml over time (in seconds) for Experiment 3.

FIGS. 31C and 31D show the data used to generate the plots of FIGS. 31A and 31B, respectively.

FIG. 32A shows the flow rate in ml per second over time for Experiment 4.

FIG. 32B shows the total volume aspirated in ml over time (in seconds) for Experiment 4.

FIGS. 32C and 32D show the data used to generate the plots of FIGS. 32A and 32B, respectively.

FIG. 33A shows the flow rate in ml per second over time for Experiment 5.

FIG. 33B shows the total volume aspirated in ml over time (in seconds) for Experiment 5.

FIGS. 33C and 33D show the data used to generate the plots of FIGS. 33A and 33B, respectively.

FIG. 34A shows the flow rate in ml per second over time for Experiment 6.

FIG. 34B shows the total volume aspirated in ml over time (in seconds) for Experiment 6.

FIGS. 34C and 34D show the data used to generate the plots of FIGS. 34A and 34B, respectively.

### DETAILED DESCRIPTION OF THE PREFERRED EMBODIMENTS

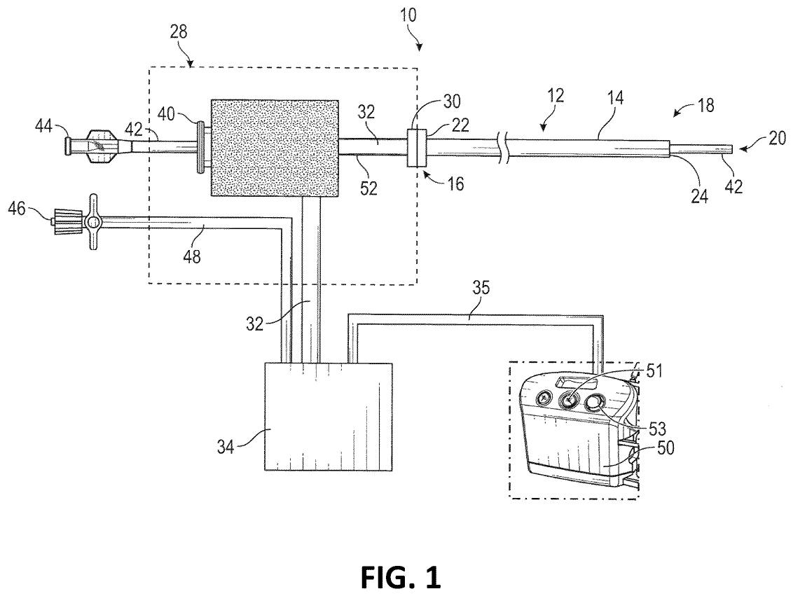

Referring to FIG. 1, there is illustrated a fluid management system for large bore aspiration procedures. The system 10 includes a large diameter first thrombectomy catheter 12, having an elongate tubular body 14 extending between the proximal end 16 and a distal end 18. A central lumen 20 extends between a proximal catheter connector 22 and a distal port 24 on the distal end 18.

In the illustrated embodiment, the catheter 12 is releasably connectable to a flow control module 28 by way of a complementary module connector 30. Module connector 30 provides a releasable connection to complementary catheter connector 22, and may include an opener (not illustrated) for opening a hemostasis valve in the hub of the large bore catheter (not illustrated).

The flow control module 28 includes a fluid flow path 32 extending between the module connector 30 and the flow control module 28. The fluid flow path 32 continues to extend between the flow control module 28 and a reservoir 34, which contains a filter for thrombus collection and/or evaluation and a chamber for filtered fluid chamber (not illustrated). In an alternate implementation, the flow control module 28 is integrally formed within the hub of thrombectomy catheter 12 to which the catheter may be non-removably attached. In addition, the flow path between the flow control module 28 and the reservoir 34 may be contained within a continuous integral tubing, or may be contained within two or more tubing components releasably connectable via complementary Luer locks or other connectors.

Flow control module 28 may include a flow regulator for regulating flow through the flow path 32. The flow regulator is configured to provide a reversible restriction in the flow path, such as by an expandable or contractible iris, a ball valve or other rotary core valve, leaf valve, a pinch tubing, or others known in the art.

In one implementation, the flow regulator comprises a collapsible portion of the tubular wall defining the flow path, such as a section of polymeric tubing. An actuator positioned adjacent the tubing is movable between a first position where it compresses the tubing, thereby restricting flow to the low flow rate, and a second position where it has moved away from the tubing, allowing the tubing to resume its full inside diameter and allow the high flow rate. The actuator may be spring biased or have other default driver in the direction of the first (restricted) position, and only movable into the second position in the presence of an affirmative mechanical force or electrical signal that actuates the high flow override. Upon removal of the momentary override command, the actuator automatically resumes the first, position, producing the low flow mode.

The actuator may be driven by a mechanical control such as a lever or rotatable knob, or an electrically driven system such as a solenoid, operated by any of a variety of buttons, levers, triggers, foot pedals or other switches known in the art, depending upon the desired functionality.

In another implementation, the fluid flow may be selectively directed through a low flow regulator such as a small diameter orifice or tube, and a high flow regulator such as a larger diameter orifice or tube. A mechanically actuated or electromechanically actuated valve can momentarily divert flow from the low flow to the high flow regulator in response to actuating a control.

Flow control module 28 thus includes one or more controls, for controlling the operation of the system. One control may be provided for toggling the system between a no flow (off) mode and a low flow mode. The same or a different control may be provided for momentarily toggling the flow regulator between the low flow mode and a momentary operator initiated high flow override mode. Release of the momentary override control causes the regulator to revert to off or low flow mode.

The low flow mode enables the first catheter 12 to approach and engage the clot with a relatively low volume of blood aspiration. Once the clot is engaged, the momentary high flow control may be activated to generate a bolus of high flow vacuum to draw the clot into the catheter 12. High flow may be at least about 10 cc/second, and preferably at least about 15 cc/sec but typically no more than about 25 cc/sec. In one construction the high flow rate is about 20 cc/sec, with all of the foregoing flow rates in an unobstructed aspiration of blood. Low flow as used herein is no more than about 50%, no more than about 35% or no more than about 25% of the high flow rate. Low flow is generally less than about 10 cc/sec or 7 cc/sec, and is often in the range of from about 1-5 cc/sec.

The flow control module 28 may be provided with a second catheter port 40 in communication with central lumen 20 via a hemostasis valve (e.g., Tuohy Borst valve) (not illustrated) within the module 28. This allows introduction of a second aspiration catheter 42 through the access catheter 12 and extending to the treatment site. The second catheter 42 may be a smaller diameter aspiration catheter, with or without clot agitation or mechanical grasping capabilities, drug delivery catheter, a mechanical disrupter or other accessory device that may be useful in the clot retrieval process. In one implementation, the second catheter including its hand piece and controls may be identical in material respects to the first aspiration catheter except the second catheter is smaller diameter and longer than the first catheter.

If desired, the second catheter 42 may be connected via a proximal connector 44 to a complementary connector 46 which is in communication with the reservoir 34 via aspiration line 48. Alternatively, aspiration line 48 may be connected to a separate aspiration and collection system (not illustrated).

The clot may be removable through the first catheter 12 under vacuum without additional assistance. However if desired, the secondary clot grasping catheter 42 may be introduced to provide additional attachment and/or mechanical disruption of the clot to facilitate removal. Removal may be assisted by the application of vacuum to the grasping catheter 42 as well as to the first catheter 12 in sequence or simultaneously depending upon the desired clinical performance.

Aspiration pump 50 may include a vacuum pump, and may also include a vacuum gauge 51, and an optional a pressure adjustment control 53. The vacuum gauge 51 is in fluid communication with the vacuum pump and indicates the vacuum pressure generated by the pump. The pressure adjustment control 53 allows the user to set to a specific vacuum pressure. Any of a variety of controls may be utilized, including switches, buttons, levers, rotatable knobs, and others which will be apparent to those of skill in the art in view of the disclosure herein. Aspiration pump 50 may alternatively be a manually activated pump such as a syringe.

Reservoir 34 is in fluid communication with the aspiration pump 50 via vacuum line 35 and acts to transfer vacuum from the air filled side of the system to the liquid side of the system, and also to collect aspirated blood and debris. Vacuum line 35 may be used as a flow restriction. Reservoir 34 thus includes a collection canister in fluid communication with flow path 32 and collects aspirated debris. The collection canister may include a filter that collects clot, which may be visually observed or accessed through a window to monitor progress of the procedure and/or used for pathologic diagnosis. The vacuum chamber and collection canister may be separate components that are in fluid communication with each other or merged within a single housing. The flow direction through the system may also be reversed to allow the blood to flow through the filter while the clot is collected outside (now downstream) of the filter, e.g. between the filter and the outer transparent window or container.

The flow path 32 extends throughout the length of the first catheter 12, through the control module 28 and into the reservoir 34. A transparent window 52 may be provided to enable direct visualization of the contents of the flow path 32. In the illustrated embodiment, the window 52 is in the form of a transparent section of tubing between the proximal end of the access catheter 12 and the flow module 28, and within the sterile field so that the clinician can directly visualize debris as it exits the proximal end of the access catheter 12 and before it reaches the reservoir 34 which may be outside of the sterile field. The actual length of the transparent tubing is preferably at least about two or four or 6 cm long and generally less than about 30 or 20 cm long. In some implementations, the length of the transparent tube is within the range of about 5 cm to about 15 cm. In an alternate implementation, the transparent window may be carried by the proximal hub of the access catheter 12, or may be a proximal portion of the catheter shaft, distally of the hub.

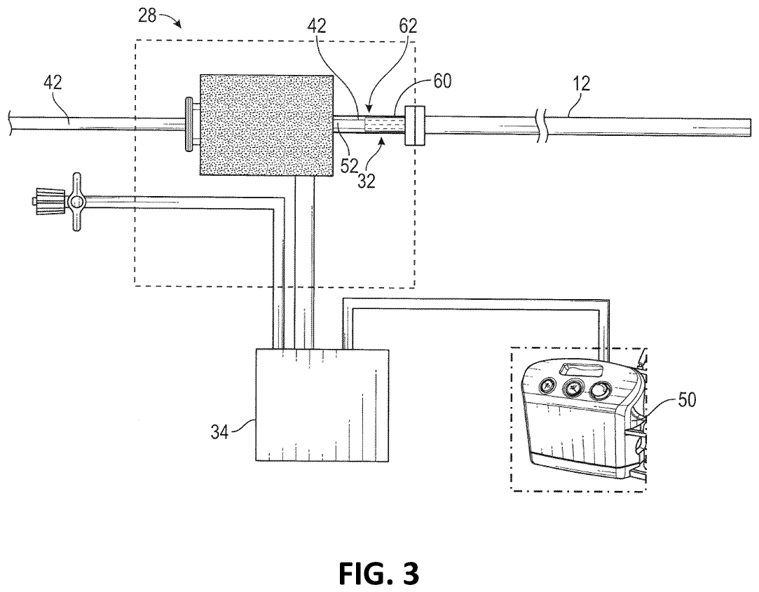

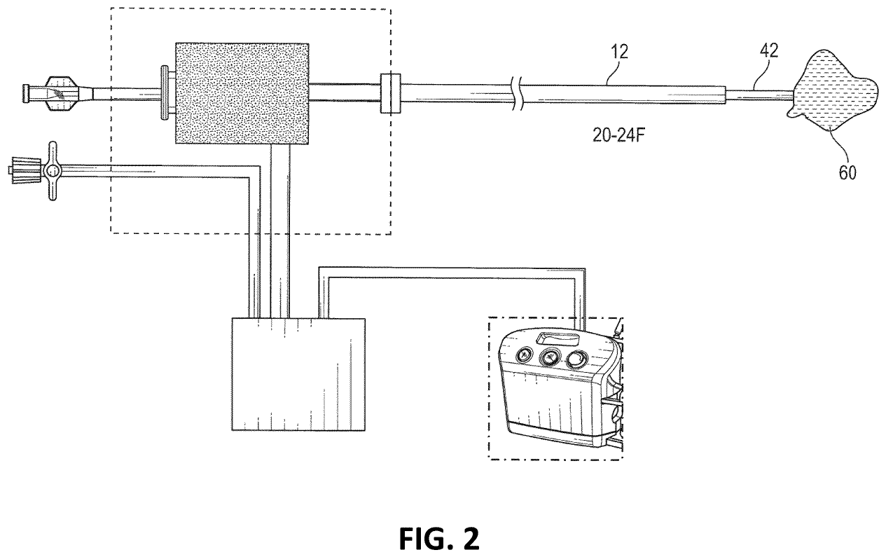

Referring to FIG. 2, the secondary catheter is in the form of a second aspiration catheter 42 which has been distally advanced through the access catheter 12 and through the vasculature into proximity with a clot 60. The clot 60 may be grasped by the second catheter 42 in any of a variety of ways such as by mechanical attachment or suction, or both.

Referring to FIG. 3, the second catheter 42 has been partially proximally retracted, drawing the clot 60 into the first catheter 12 such that the clot 60 becomes visible through the window 52. This may be facilitated by applying vacuum through both the grasping catheter 42 and the access catheter 12.

Continued proximal retraction of the grasping catheter 42 brings an interface 62 between the grasping catheter 42 and the clot 60 into view through the window 52. This enables the clinician to visually confirm that a clot has been captured.

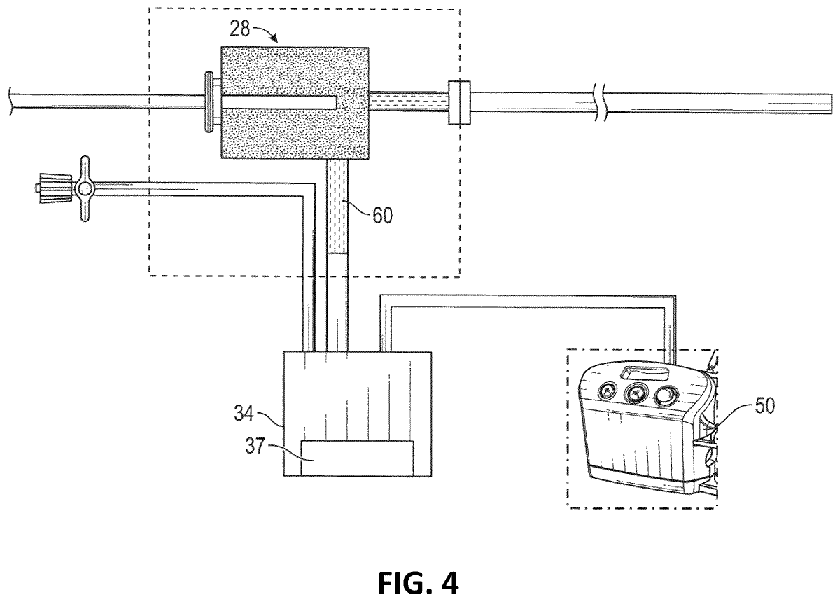

Referring to FIG. 4, further proximal retraction of the grasping catheter 42 allows the clot 60 to be drawn through the flow path 32 in the direction of the reservoir 34. The clot 60 is there after drawn by vacuum into the collection chamber within reservoir 34, where it may be captured by a filter and viewed through a transparent sidewall or window 37 on the collection chamber.

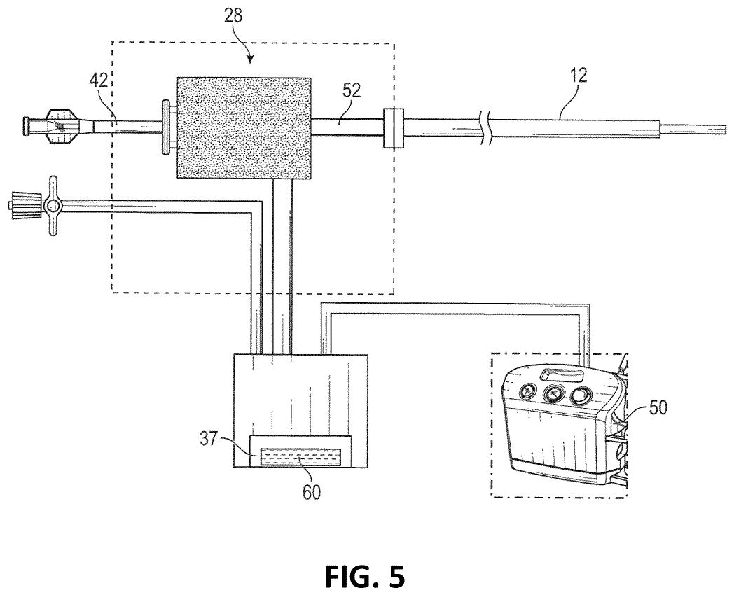

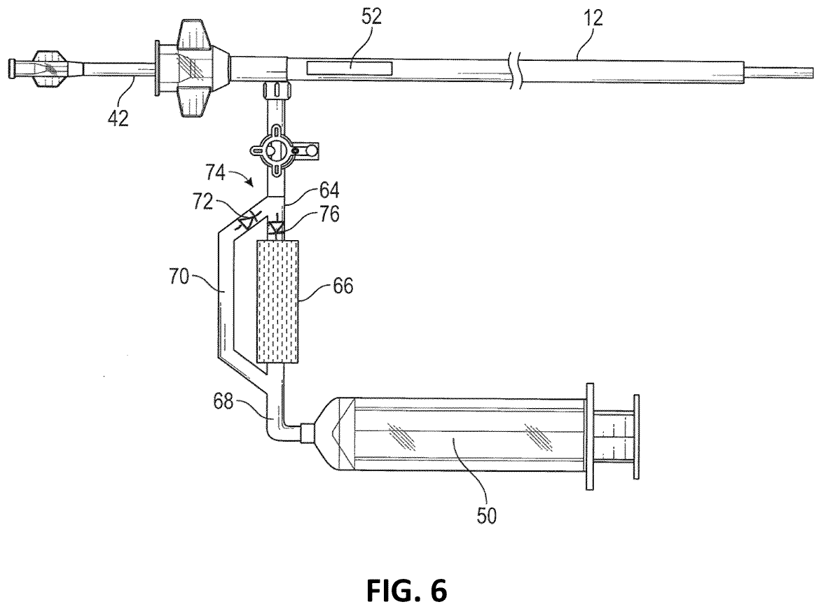

Another aspect of fluid management during the thrombectomy procedure is illustrated in FIG. 6. In this implementation, an aspiration line 64 places the first catheter 12 in communication with a thrombus filter 66. The thrombus filter 66 is further in communication with a pump such as a syringe aspiration pump 50 by way of aspiration line 68. Actuation of the pump 50, such as by proximally retracting the plunger, draws thrombus through the access catheter 12 and into the thrombus filter 66 where thrombus and thrombus particles having a size greater than a predetermined threshold will be entrapped. The thrombus filter 66 may be provided with a transparent window for a visual confirmation, as has been discussed.

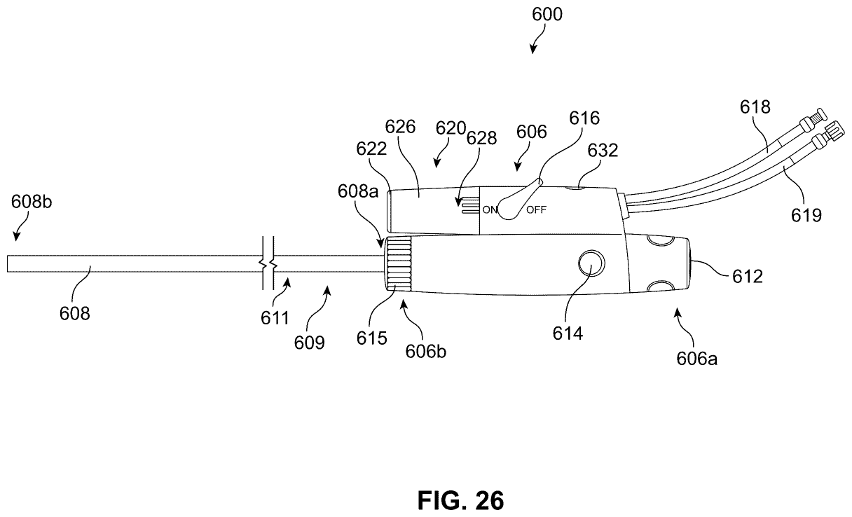

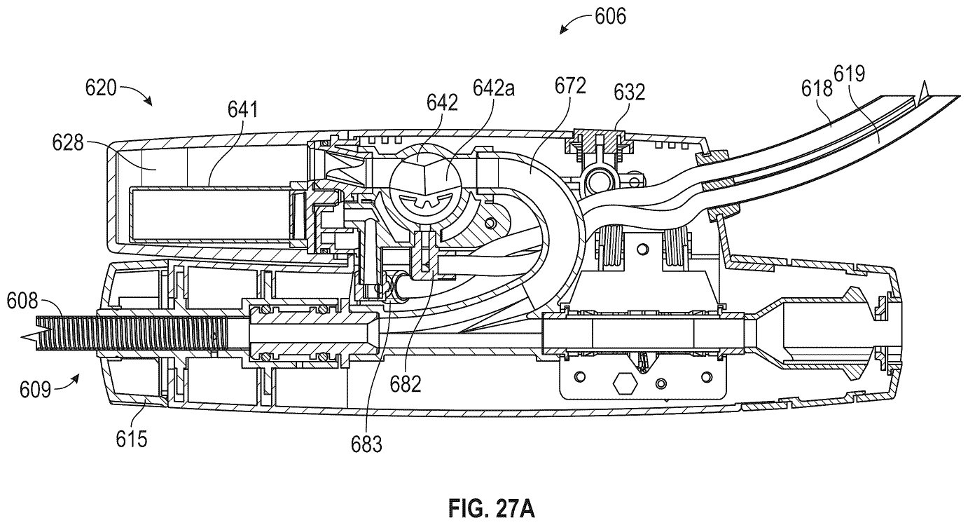

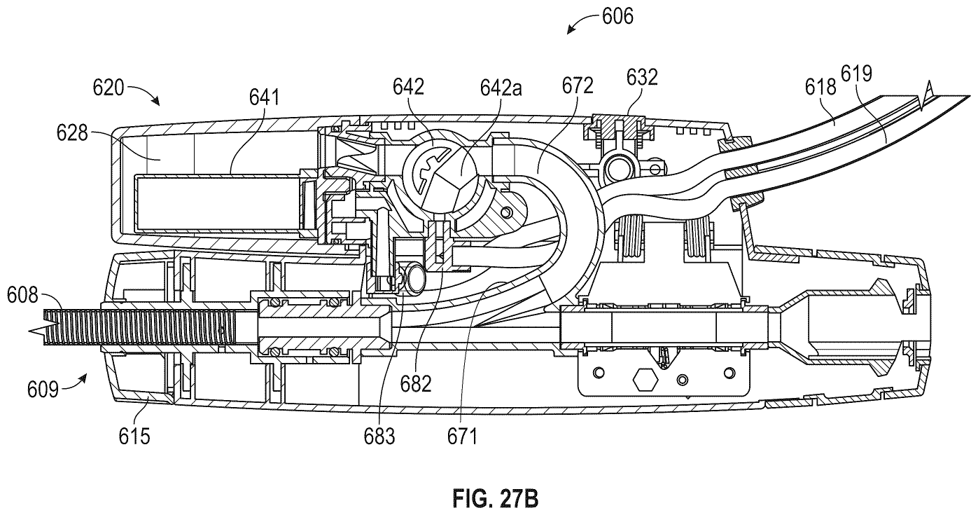

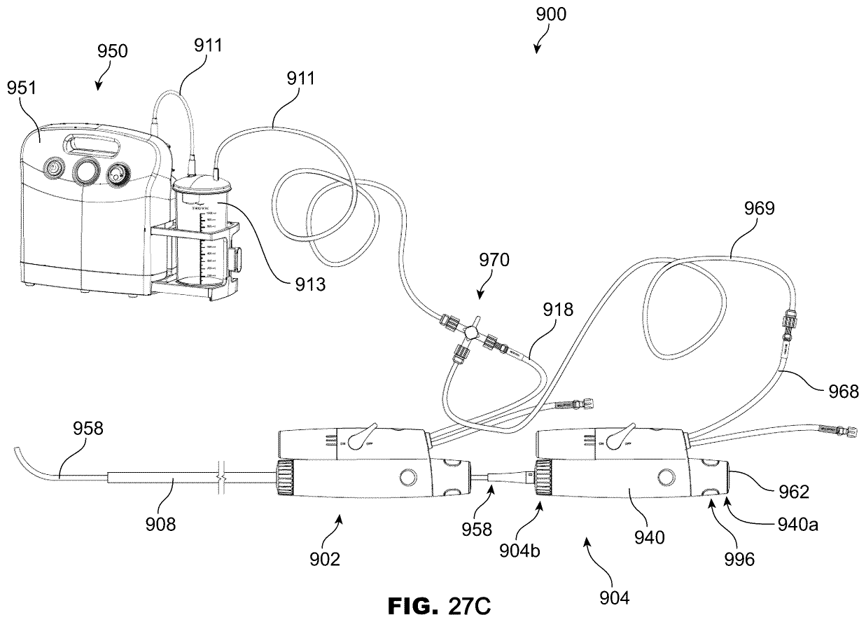

Blood drawn into the syringe 50 will therefore be filtered, with the debris remaining in the thrombus filter 66. Blood in the pump 50 or other reservoir downstream from the filter may be re-infused into the patient. In the illustrated configuration this may be accomplished by reversing the pump (pushing the plunger) and pushing filtered blood via a bypass tube 70 which merges with the flow path 32 on the patient side of the filter 66 and back into the patient. In some cases, the blood in the pump 50 or other reservoir downstream from the filter 66 may be re-infused into the patient via an introducer sheath and/or through a multiport, such as the multiport 619, which is described in relation to FIG. 26 and FIGS. 27A and 27B. A valve assembly 74 is preferably provided to direct thrombus containing blood from the patient into the filter 66 but ensure that only filtered blood can be pumped through bypass 70 and back into communication with the flow path 32 and into the patient.

In the illustrated implementation, the valve assembly 74 comprises a first valve 72 in the bypass tube 70 which permits flow of filtered blood in the direction of the patient but blocks the flow of unfiltered blood through the bypass tube 70 in the direction of the pump 50. The second valve 76 is provided to permit flow of unfiltered blood in the direction of the filter 66 but prevent the flow of blood from the filter back in the direction of the patient. In one implementation, the first valve 72 and second valve 76 are one way flapper valves that open or close in response to blood flow direction.

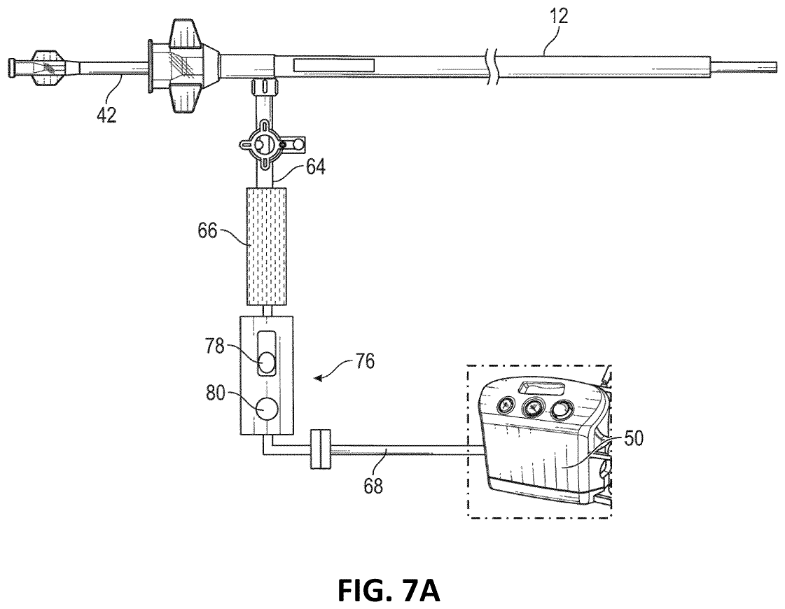

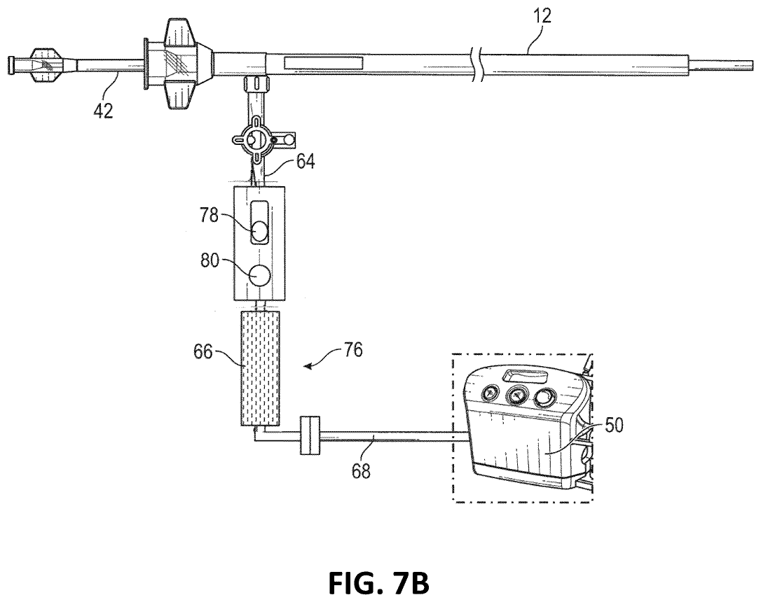

A further configuration of the fluid management system is schematically illustrated in FIG. 7A. Aspiration line 64 places the first aspiration catheter 12 in communication with the thrombus filter 66. The thrombus filter 66 is in communication with the aspiration pump 50 by way of aspiration line 68. Aspiration line 68 includes a flow control 76. Flow control 76 includes an off/on control such as a switch 78. Activation of the switch 78 to the ‘on’ configuration places the system in a low flow vacuum mode as has been discussed. Activation of a momentary full flow control such as a button 80 changes the system to the high flow mode.

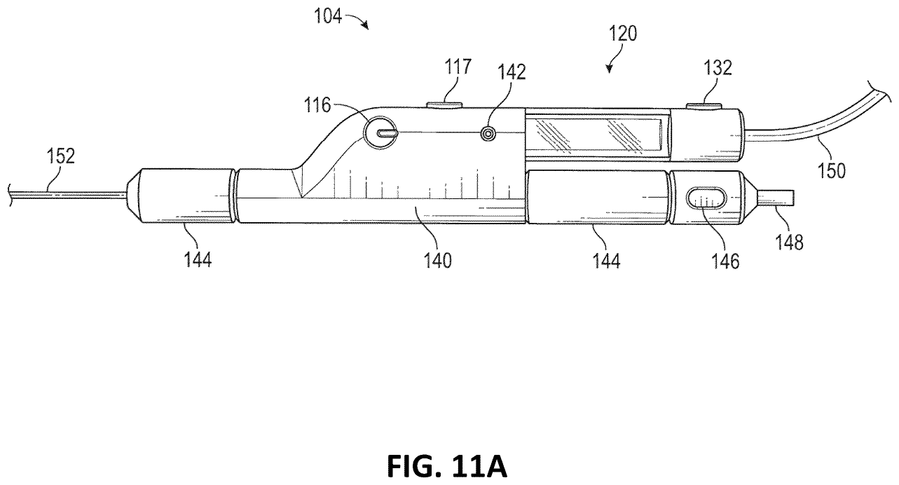

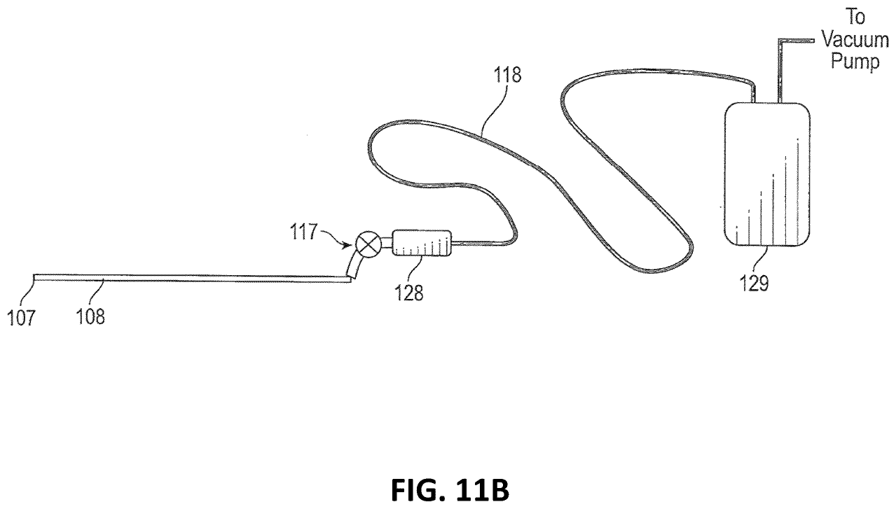

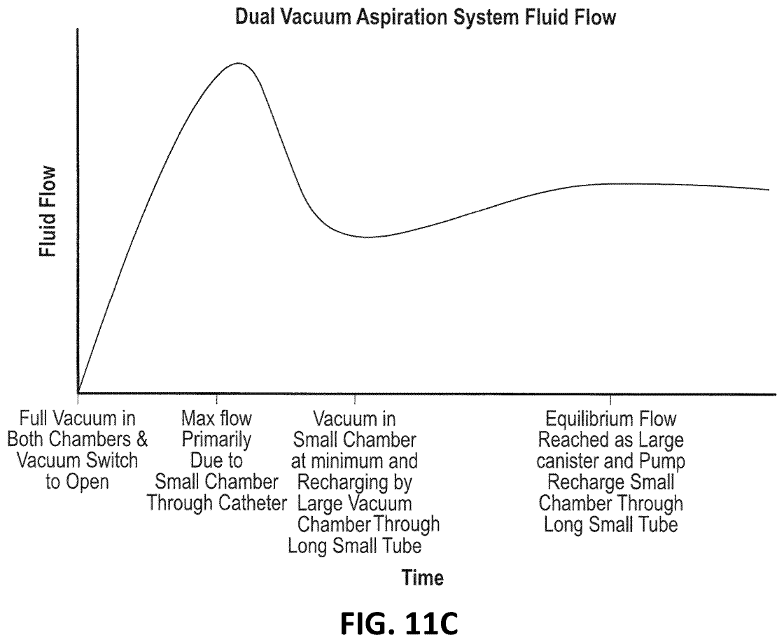

In an alternate configuration illustrated in FIG. 7B, the flow control 76 is moved from between the aspiration pump 50 and thrombus filter 66 to in between the catheter and the thrombus filter 66. This allows the negative pressure in the chamber of thrombus filter 66 to reach equilibrium with the canister in the aspiration pump 50 when the valve in flow control 76 is closed. When the valve is subsequently opened, the relatively short distance between the thrombus filter and the patient allows a rapid drop in negative pressure at the distal end of the catheter as is discussed in greater detail in connection with FIG. 11B. The flow control 76 my additionally be provided with an optional vent to atmosphere, or to no vacuum, or vent to a source of vacuum at a milder vacuum than that experienced in the cannister of the aspiration pump 50.

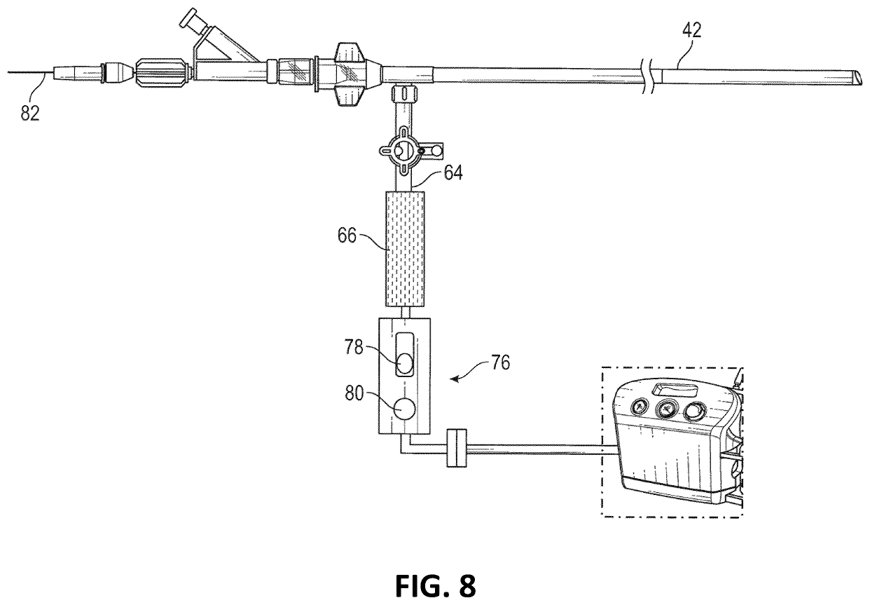

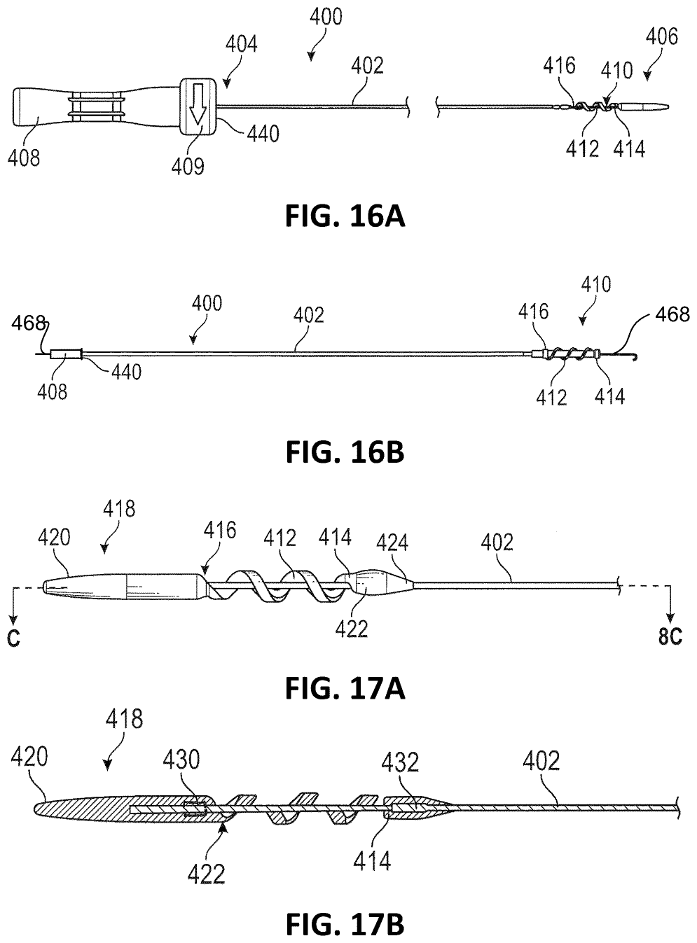

FIG. 8 illustrates a second, smaller aspiration catheter 42 such as a 16 French catheter, configured for the application of suction to facilitate grasping a clot. In a typical configuration, the second catheter 42 will be extended through a first, larger catheter 12 (not illustrated) as has been discussed. As with any of the second catheters disclosed here in, a mechanical agitator 82 may be axially movably positioned within a central lumen of the grasping catheter 42. See also FIGS. 16A-18B. Additional details of one suitable mechanical agitator 82 are disclosed in U.S. Pat. No. 10,653,434 to Yang, et al., entitled Devices and Methods for Removing Obstructive Material from an Intravascular Site, the entirety of which is hereby expressly incorporated herein by reference. Additional details of the mechanical agitator 82 are disclosed in U.S. Pat. No. 10,835,711, to Yang et al., issued Nov. 17, 2020, entitled Telescoping Neurovascular Catheter with Enlargeable Distal Opening, and U.S. Pat. No. 10,835,272, to Yang et al., issued Nov. 17, 2020, entitled Devices for Removing Obstructive Material from an Intravascular Site, the entireties of which are hereby expressly incorporated herein by reference.

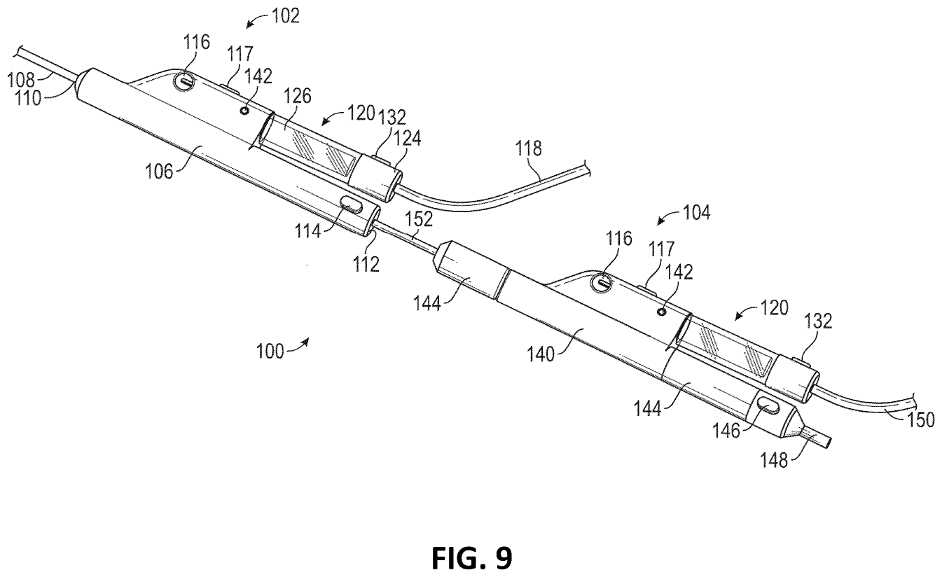

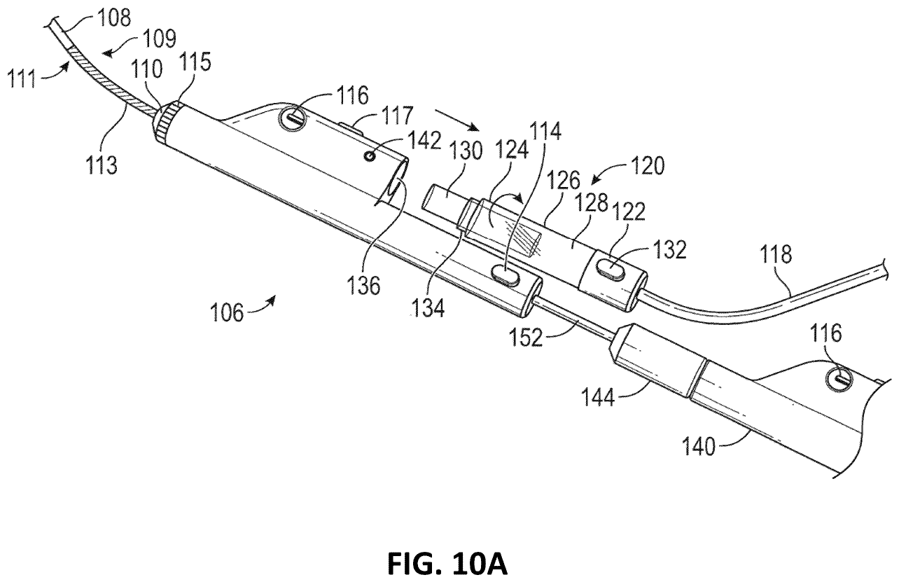

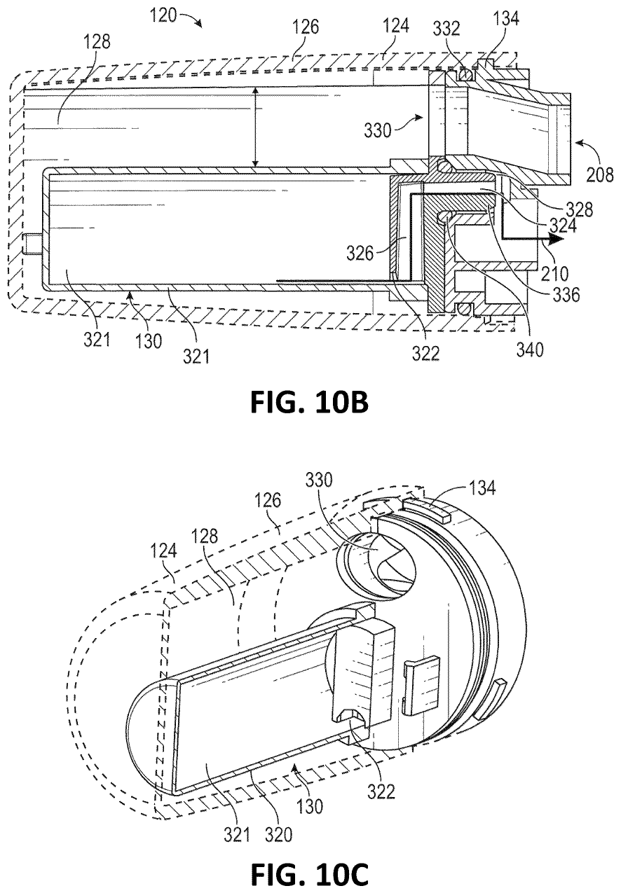

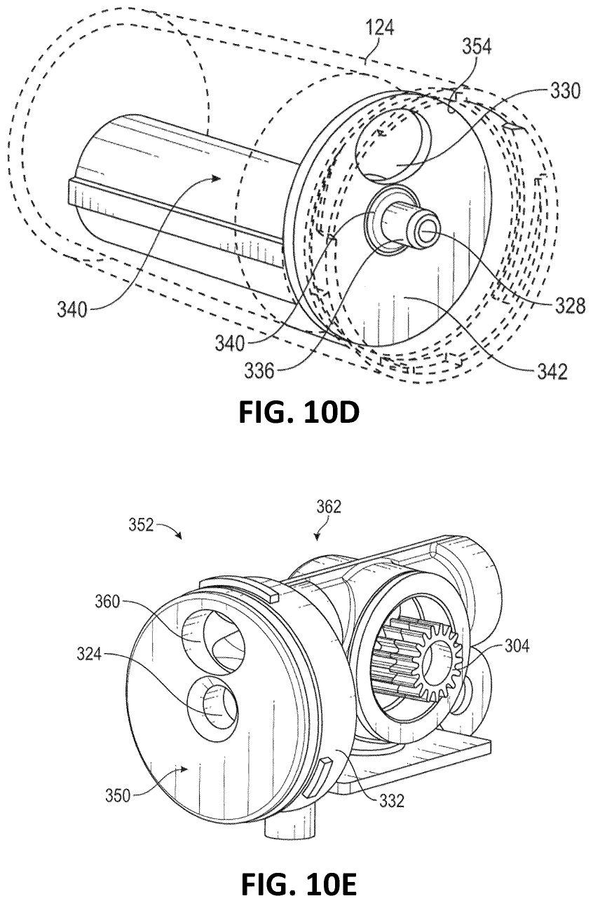

Referring to FIGS. 9 and 10A, there is illustrated a further implementation of an aspiration system 100. The system includes a first thrombectomy catheter 102, such as a large bore aspiration catheter, and a second aspiration catheter 104 which is optionally advanceable through the first thrombectomy catheter 102 as has been discussed, or used by itself.

Thrombectomy catheter 102 comprises a proximal handle 106 having an elongate flexible tubular catheter body 108 extending distally therefrom. The proximal end 110 of the tubular body 108 may be permanently carried by the proximal handle 106 or may be provided with a releasable connector for detachable connection to a complementary connector on the handle 106.

In one implementation, the tubular body 108 or 152 or both are provided with a flexible neck 109 extending between proximal end 110 and a transition 111. The flexible neck 109 has a greater flexibility than the adjacent portion of the tubular body 108 distal to the transition 111. The flexible neck 109 may have a length of at least about 2 cm and often at least about 4 cm, but generally no more than about 20 cm or 10 cm or less.

The sidewall of the catheter body 108 within flexible neck 109 includes a helical coil 113 having adjacent filars spaced apart to both improve flexibility, and also allow visualization between adjacent windings of the coil. At least the flexible neck 109 includes a sidewall window such as the spaces between adjacent coil windings which may be in the form of an optically transparent outer tubular layer, such as any of a variety of optically transparent shrink tubing polymers. This allows visualization of clot through the side wall as it passes through the neck 109 before it enters the proximal handle. The transparent window on the larger catheter 108 also allows visualization of the distal tip of the inner catheter 152 as it passes the window. This may be facilitated by placing a visual marker on the distal end of the inner catheter 152 such as a colored annular band.

For example, in an implementation having a 24 French tubular body 108, the smaller tubular body 152 (e.g. 16 French catheter) may be provided with a visual indicium such as a white tip on the distal end, that can be visualized through the sidewall window as it passes through the flexible neck 109. The flexible neck 109 may also be provided on the catheter shaft 152.

The spring coil 113 may extend distally to a point of termination within about one or 2 cm of the transition 111, and, and one implementation, at the transition 111. Distally of the transition, the sidewall of tubular body 108 may include a tubular braid, importing greater stiffness and higher push ability than the helical coil 113.

The proximal end of the catheter may be provided with a rotation control such as a rotatable knob 115 which may be rotationally fixed to the catheter and rotatable with respect to the handle housing. This facilitates relative rotation between the catheter and the housing for any of the large or small bore catheters disclosed herein.

A central lumen extending through the tubular catheter body 108 is in communication with a flow path extending through the proximal handle 106 to a proximal access port 112. The flow path between the tubular catheter body 108 and the proximal access port 112 is preferably linear, to axially movably receive the second catheter 104 which may or may not be utilized in a given procedure. To accommodate the absence of second catheter 104 and seal the port 112, the proximal handle 106 is preferably provided with a homeostasis valve 114 such as a Tuohy-Borst valve.

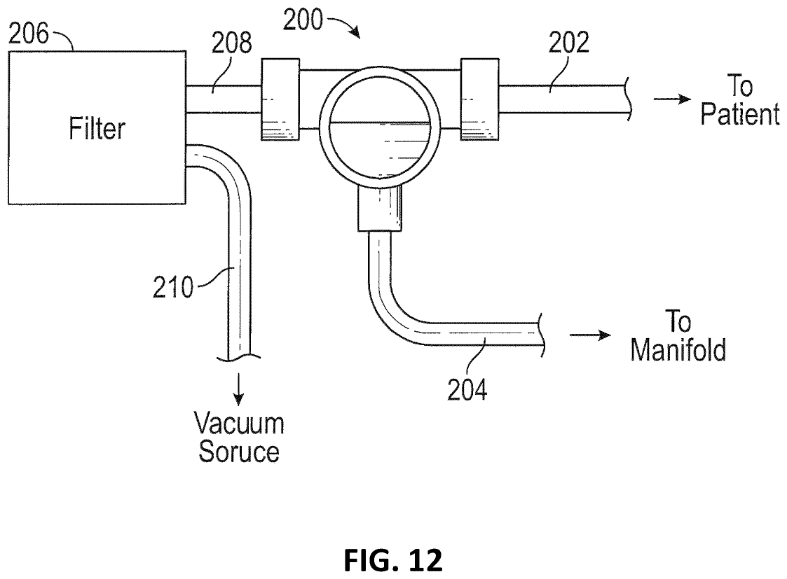

A manifold switch 116 controls two way or three way a manifold valve (illustrated in FIG. 12) for selectively controlling fluid flow as discussed further below. An aspiration control 117 is provided to turn aspiration on and off. Alternatively, manifold switch 116 can be configured to turn aspiration one and off.

A filter assembly 120 includes housing 122 with a side wall 124, at least a portion of which includes a transparent window 126. Window 126 permits a viewing of the contents (e.g. aspirated clot) of a filter chamber 128, which contains a filter 130.

The filter assembly 120 is configured to place the filter 130 in the flow path between the tubular catheter body 108 and the aspiration tubing 118. Preferably the filter chamber can be closed to maintain negative pressure conveyed from a pump via aspiration tubing 118, or opened to permit insertion or removal of the filter 130. In the illustrated implementation, the filter assembly 120 is removably connected to the handle 106. A connector 134 such as a first thread on the housing 122 is releasably engageable with a complementary connector 136 such as a complementary thread on the handle 106. A vent (aperture) to atmosphere may be provided in communication with the filter chamber, to reduce foaming of blood in response to reduced pressure.

The present implementation includes an integrated flow control module in the proximal handle 106. Thus, an adjustable flow regulator (not illustrated) may be positioned in the flow path, to enable controllable toggling of the aspiration between a low flow mode and a high flow mode. In the illustrated implementation, optional flow regulator is positioned downstream of the filter 130, and contained within the housing 122 of the filter assembly 120. A flow regulator control 132 is provided, to control the flow rate. Preferably, as has been discussed, the flow regulator is configured to regulate fluid flow through the flow path at a default low flow rate. Activation of the flow control 132 adjust the flow to the high flow rate mode. Flow control 132 may be a momentary button, slider switch, trigger, knob or other structure that is preferably defaulted to the low flow mode.

In any of the catheters disclosed herein, carrying the filter chamber 128 on the catheter or at least spaced apart from the remote vacuum pump and vacuum cannister provides enhanced aspiration performance. The location of a conventional aspiration pump may be far enough away from the patient to require a length of aspiration tubing between the pump and the catheter to be as much as 50 inches or 100 inches or more (for example, 106 inches). The pump typically includes an aspiration canister for blood collection. When aspiration is desired, a valve is opened to place the low pressure cannister in communication with the catheter by way of the aspiration tubing, to aspirate material from the patient. But the length of the aspiration tubing operates as a flow restrictor, causing a delay between the time of activating the vacuum button and actual application of suction to the clot.

In accordance with one aspect, the catheter handle 106 or 140 contains a filter chamber 128 for example, which is in communication with the vacuum cannister on the pump by way of elongate aspiration tubing 118. The momentary aspiration control 117 is in between the filter chamber 128 and the catheter, which, in the default off position, allows the entire length of the aspiration tubing 118 and the filter chamber 128 to reach the same low pressure as the aspiration cannister on the pump. The flow restriction between the pump cannister 129 and the filter chamber 128 is greater than the flow restriction between the filter chamber 128 and the patient.

In alternate configurations, 117 may be a vent to atmosphere which allows the clot canister to be evacuated. Element 142 can alternatively be an injection port such as for injecting contrast media, saline, or drugs.

Thus, the only remaining flow restrictor between a source of vacuum (filter chamber 128) and the patient is the relatively short aspiration pathway between the valve in the handpiece and the distal end of the catheter. When the momentary aspiration control 117 is activated, the flow restriction and enclosed volume on the patient side of the filter chamber is low relative to the flow restriction and enclosed volume through aspiration tubing 118 on the pump side of the filter chamber 128.

This dual chamber configuration produces a rapid spike in negative pressure experienced at the distal end of the catheter upon activation of the aspiration control 117. The response time between activating the aspiration control 117 and realizing suction actually experienced at the clot is significantly faster and allows significantly higher initial flow than the response time realized in a conventional system having only a vacuum chamber located at the pump.

The spike of negative pressure experienced at the distal end of the catheter will fade as pressure equilibrium is reached between the filter chamber and canister. When the momentary aspiration control 117 is closed, the vacuum pump will gradually bring the pressure in the filter chamber 128 back down to the level in the vacuum cannister at the pump.

A simplified fluid flow diagram is illustrated in FIG. 11B, and a qualitative flow rate diagram is illustrated in FIG. 11C. The flow restriction between chamber 128 and the distal and 107 of catheter 108 is small relative to the flow restriction between the vacuum canister 129 and the vacuum chamber 128. This allows a negative pressure peak experienced at distal end 107 almost instantaneously upon activation of vacuum switch 117. The flow rate of material into the catheter 108 rapidly reaches a peak and subsides as vacuum chamber 128 fills with aspirated material. The vacuum in chamber 128 declines to a minimum, and slowly recharges by the large vacuum chamber 129 and associated pump through tubing 118. In use, a clinician may choose to allow the momentary vacuum switch 117 to close at or shortly following the maximum flow rate, just giving a short burst or spike of vacuum to facilitate spiration of thrombus into the catheter 108.

Additional details of the filter assembly and related structures are illustrated in FIGS. 10B to 10E. Referring to FIG. 10B, the filter assembly 120 includes a tubular sidewall 124 having a transparent window 126. In some implementations the entire tubular sidewall 124 can be a transparent window. The side wall 124 encloses a filter 130 as has been discussed. The filter 130 includes a tubular filter sidewall 320 defining an interior chamber 321 for filtered blood. Filtered blood is drawn in the direction of vacuum line 210 through a first vacuum aperture 322 and into a flow path 324 having a vertical offset 326 in the flow path 324. The vertical offset 326 allows removal of blood from the bottom of the chamber, through a flow path and out through a second vacuum aperture more centralized with respect to a central axis of the tubular sidewall 124 and in communication with vacuum line 210.

The filter 130 is displaced downward with respect to a central longitudinal axis of the tubular sidewall 124, leaving the filter chamber 128 having a chamber height 129 at least as great as the inside diameter of a filter line aperture 330 leading to filter line 208. This allows clot to move from filter line 208 into the filter chamber 128 without restriction, and optimizes the volume of filter chamber 128 on top of the filter 130 for viewing through the window 126.

A connector 134 maybe carried by the filter assembly 120, such as in the form of a bayonet mount, or other releasable attachment to the handpiece housing. A first seal 332 such as an annular elastomeric ring may be provided between the tubular sidewall 124 and the complementary surface on the handpiece housing.

A second vacuum aperture 328 is in communication with the first vacuum aperture 322 by way of the flow path 324. Second vacuum aperture 328 may be carried on an axially extending tubular projection 336 which may be removably received within a complementary recess on the hand piece housing.

A second seal 340 such as an elastomeric ring maybe provided surrounding the flow path 324, for providing a seal between the filter assembly and the handpiece. In the illustrated implementation, the second seal 340 surrounds the tubular projection 336 and is configured to seal against an adjacent complementary surface on the handpiece in the as mounted orientation.

Referring to FIG. 10D, the filter assembly 120 additionally includes a filter base 342 through which filter line aperture 330 extends. The flow path 324 additionally extends through the filter base 342, and, in the illustrated implementation, exits the tubular projection 336 carrying the second vacuum aperture 328.

A complementary docking platform 350 is carried by the handpiece, having complementary connector to connector 134 for rapid attachment and detachment of the filter assembly 120 from the handpiece. In the illustrated embodiment, at least a first flange 352 maty be received through an opening 354 on the filter assembly 120. Rotation of the filter assembly 120 moves the first flange into interference fit with a second flange 356 to secure the filter assembly 120 to the docking platform 350 on the handpiece. Two or three or four or more similar flange and complementary opening pairs may be provided around the periphery of the components. In the illustrated implementation, the circumferential arc length of the flange and corresponding opening on one of the three pairs is greater than the other two pairs to function as a key, so that the filter assembly can only be secured to the docking platform in a single rotational orientation.

The docking platform 350 includes a filter line aperture 360 for communicating with filter line 208, and a vacuum line aperture 362 for placing the filter 130 in communication with a source of vacuum. The docking platform 350 may be connected to a two way valve 362 or a three way valve as is discussed elsewhere herein depending upon the desired functionality. The valve may carry a rotatable drive gear 304 to rotate the interior rotatable valve gate as is discussed in additional detail below. Alternatively, a lever or other control on the housing may be configured to rotate a shaft directly coupled to the rotatable part of the valve.

A valved flow path may also be provided for venting the filter chamber 128 directly to atmosphere. The valve may be opened such as by depressing a momentary button, which is biased in the closed direction. This can create an abrupt change in pressure at the distal end of the catheter, which may facilitate clot aspiration. This can also be used to discharge vacuum

Referring to FIG. 11A, additional details of the handle 140 of the second catheter 104 are disclosed. The handle 140 extends between a proximal end and a distal end. An elongate flexible tubular body 152 extends distally from the distal end of the handle 140 and is configured to advance distally through the proximal handle 106 and the tubular body 108 of thrombectomy catheter 102.

A steering dial 144 may be provided to place one or more steering wires under tension, to deflect a deflection zone near the distal end of the tubular body 152. A manifold switch 116 may be provided to control the flow of fluid as will be discussed below. The handle additionally comprises an aspiration control 117 such as a slider switch, for turning aspiration on or off. A max button 132 may be provided for delivering a momentary pulse of high aspiration rate as has been discussed.

Fluid flow through the thrombectomy system is controlled by manifold switch 116 (see, e.g., FIG. 9), which may control a two way or three-way valve. Referring to FIG. 12, a schematic flow diagram for three-way valve 200 is provided. Patient line 202 can be placed in fluid communication with the patient, via a catheter such as a large diameter thrombectomy catheter 12 or second catheter 42.

Patient line 202 may be placed in communication with a manifold line 204 by advancing the three-way valve 200 to a first position, such as to allow delivery of medications, contrast media or saline to the patient.

Adjustment of the three-way valve 200 to a second position can isolate patient line 202 and place the manifold in communication with the filter 206 via filter line 208. Activation of a vacuum pump will draw blood from the patient and through the filter 206 via vacuum line 210.

Further adjustment of the three-way valve 200 to a third position will place the manifold in communication with the vacuum line 210, such as to permit a saline flush of the filter 206. This third position may be eliminated depending upon the desired functionality.

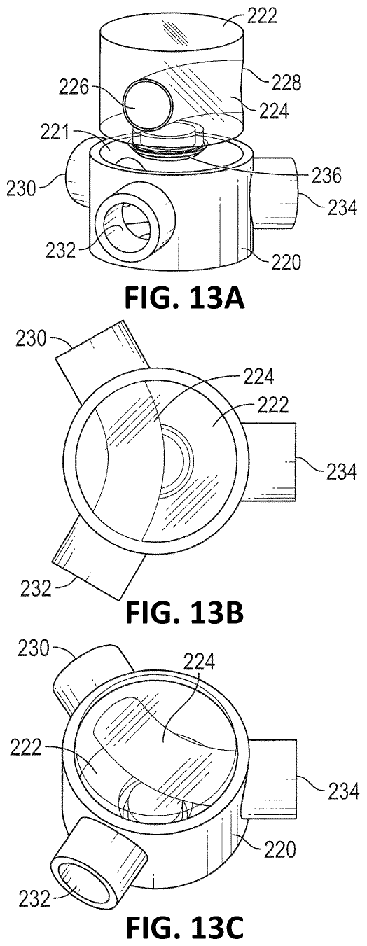

One implementation of a suitable three-way valve 200 is illustrated in FIGS. 13A through 13C. Referring to FIG. 13A, the valve 200 may comprise a housing 220 such as a cylindrical housing having a central cavity 221. A rotatable cylindrical gate 222 may be positioned in the central cavity 221, as illustrated in the exploded view of FIG. 13A. Rotatable gate 222 is provided with a flow path 224 extending between a first end 226 and a second end 228. In the illustrated implementation, the first end 226 and a second end 228 of the flow path are spaced apart around the circumference of the rotatable gate by approximately 120 degrees.

In the rotational orientation of the rotatable gate 222 illustrated in FIG. 13A, the first end 226 of the flow path 224 is in communication with a first port 232, and the second end 228 of the flow path 224 is in communication with a second port 234. This corresponds to the first position discussed previously, in which the patient is in fluid communication with the manifold.

FIG. 13B illustrates rotatable gate 222 in the second position where the flow path 224 places the first port 232 in communication with the third port 230 to place the filter 206 in communication with the manifold. The rotatable gate 222 may be toleranced within the cavity 221 such that the rotatable gate 222 seals the second port 234 thus isolating the patient from the flow path in this orientation. Similarly, in each of the other two orientations, two of the ports are placed in communication with the flow path, while the third port is isolated from the flow path.

The third position is illustrated in FIG. 13C, in which the flow path places the second port 234 in communication with the third port 230, placing the filter 206 in communication with the patient, and isolating the manifold from the flow circuit.

The foregoing selectivity may be achieved by spacing the three ports approximately 120 degrees apart around the circumference of the housing, to cooperate with the flow channel 224 end ports which are about 120 degrees apart around the circumference of the cylindrical gate 222. The gate 222 may be rotated within the housing 220 by a connector 236 extending through the housing 220 such as along the axis of rotation, and connected to a control 116 such as a rotatable knob, lever or slider switch with a rack and pinion drive assembly.

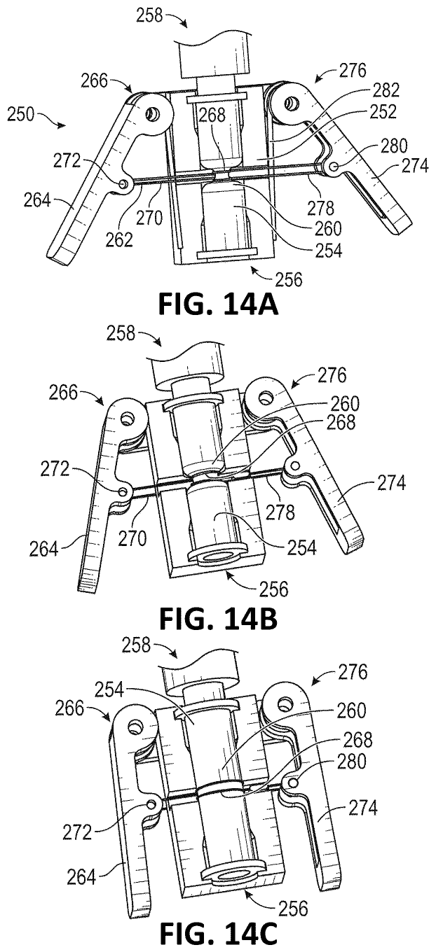

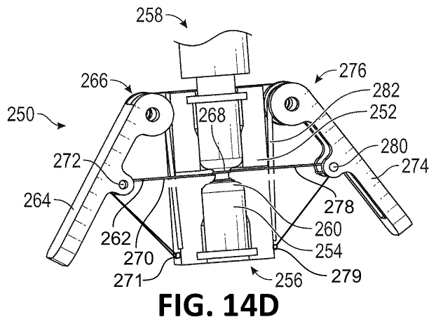

Each of the catheters disclose herein may be provided with a hemostasis valve on the proximal end, to allow selective closing of the central lumen to completely closed without any devices extending therethrough, from a sealed fit around devices of differing diameters such as a guide wire or a secondary catheter extending therethrough. One example of a suitable hemostasis valve is schematically illustrated in FIGS. 14A through 14C.

Referring to FIG. 14A, hemostasis valve 250 includes a frame 252 for supporting a flow path defined within a tubular sidewall 254. The frame 252 may be integrally formed with or mounted to the catheter handle or hub.

The flow path and tubular sidewall 254 extend between a first end 256 and a second end 258. First end 256 may be a port 112 (see, e.g., FIG. 9) on the proximal end of any of the catheters disclosed herein. Second end 258 may be in communication with the central lumen of the corresponding aspiration catheter, such that devices entering the first end 256 and advanced axially through the flow path can advance all the way to the distal end of the aspiration catheter and beyond.

At least a portion 260 of the sidewall 254 is collapsible in response to external pressure. That portion 260 and optionally the full length of the tubular sidewall within valve 250 may be comprise a collapsible elastic tube such as silicone tubing, which is biased into an open lumen tubular configuration when unconstrained. A compression element such as filament 262 is configured to apply compressive force against the sidewall 254 to reduce the inside diameter of the flow path to provide a seal against itself (when completely closed with no devices extending therethrough) or against a device such as a guidewire or catheter extending therethrough. In the illustrated implementation, the filament 262 forms a loop 268 around the collapsible portion 260 of tubular sidewall 254. Retraction of a first tail portion 270 of the filament 262 away from the sidewall 254 constricts the diameter of the loop 268 thereby collapsing the portion 260 of the tubular sidewall as illustrated in FIG. 14A.

In the illustrated implementation, the first tail portion 270 of the filament 262 may be retracted by at least a first lever 264. Lever 264 may be connected to the frame 252 by a first pivot 266 and is attached to the tail portion 270 at an attachment point 272. Advance of the lever in a first direction places the filament under tension and reduces the inside diameter of the valve. Releasing the lever removes the tension and the collapsible portion 260 of the sidewall rebounds to its unconstrained, open lumen configuration.

In the illustrated implementation, a second lever 274 is attached to the frame 252 at a second pivot 276, and is attached to a second tail portion 278 of the filament 262. Each of the first and second tail portions may comprise a single filament or two or three or more parallel filaments. In the two filament configuration as illustrated, the filaments may be immovably secured to the lever, or may be a continuous filament, looped around a fulcrum 280. The loop 268 may comprise one or two or three or more revolutions around the tubular sidewall, depending upon the desired performance.

At least one lever 264 is provided with a spring 282 to bias the lever away from the tubular sidewall, constricting the inside diameter of the collapsible portion 260 into sealing engagement with a device extending therethrough, or to a completely closed configuration in the absence of a device. As illustrated, a second lever 274 may also be biased using the same spring or a second spring.

As illustrated in FIG. 14C, compression of the levers in a medial direction towards the axis of the tubular sidewall 254 releases tension on the tail portions of the filament and allows the valve to open, such as to permit advance of a catheter through the valve. Releasing the levers allows the spring bias to retract the tail portions, reducing the diameter of the loop 268 and collapsing the collapsible portion 260 into sealing engagement with the outside surface of the secondary catheter, at an intermediate valve diameter as seen in FIG. 14B.

Retraction of the tail portion 270 of filament 262 may alternatively be accomplished by winding the tail portion 270 around a rotatable spool such as a shaft or drum. Rotation of a knob or advance of a lever causes the spool to take up filament and collapse the sidewall.

An alternate configuration for the filament 262 is illustrated in FIG. 14 D. In this implementation, the first tail portion 270 slidably extends around a first fulcrum at 272 and returns to attach to the housing at an attachment point 271. First tail portion 270 extends from the fulcrum to form a loop 268 around the collapsible tube. The filament 262 may make a single revolution or two or more revolutions around the collapsible tube before continuing on around a second fulcrum at 280, to a second point of attachment 279 to the housing.

Compression of the first lever 264 and second lever 274 loosens the loop 268, allowing the lumen to resume patency. Releasing the levers allows the spring bias to reduce the diameter of the loop 268 as the first tail portion 270 and second tail portion 278 slide away from each other around the left and right fulcrums. Preferably, friction between the filament 262 and fulcrums are minimized, as by providing a lubricious oil such as silicone oil around the fulcrums at 280 and 272, as well as using Teflon braided line for the filament 262.

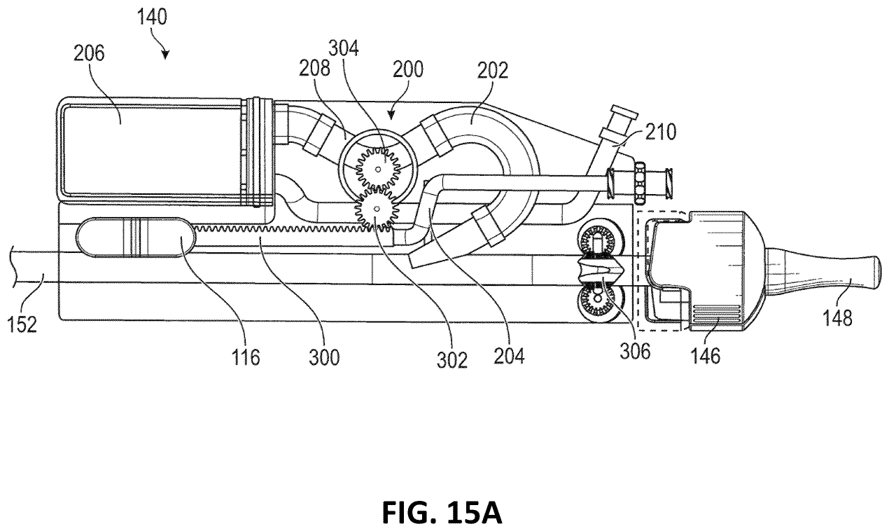

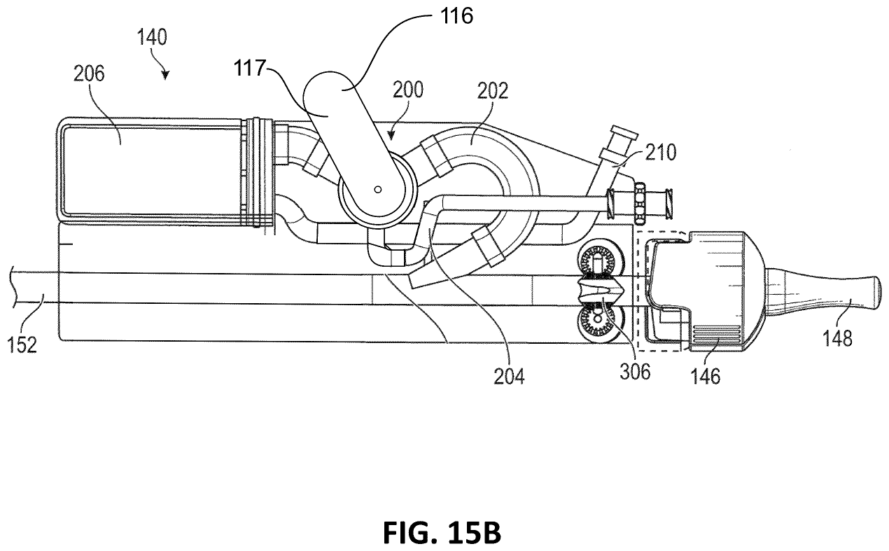

Various components of the aspiration system handle are schematically represented in context in FIG. 15A. The proximal handle 140 on a second catheter 104 includes a filter 206, a tubular body 152 and other features previously described. Two-way or three-way valve 200 selectively controls flow among the filter line 208, patient line 202 and manifold line 204. In this implementation, the three-way valve control 116 is in the form of the slider switch. The slider switch axially movably displaces a first linear rack gear 300. Rack gear 300 engages a pinion gear 302, which may either directly rotate the gate in the valve 200, or, as illustrated, drive a third gear 304 which rotates the rotatable gate within 200. An alternative valve control system is schematically illustrated in FIG. 15B. In this implementation, the slider switch, linear rack gear 300 and pinion gear 302 omitted. A valve control 116 in the form of a lever 117 is attached directly to a shaft which controls rotation of the valve gate. The lever may be advanced proximally or distally, to adjust the flow path through the valve as has been discussed.

A steering mechanism 306 is provided to permit steering of the second catheter 152. Manually rotatable knob 148 allows manual rotation of a core wire and distal helical tip as has been discussed. The core wire axially movably extends across hemostasis valve 146. Alternatively, the core wire and tip (e.g., thrombus engagement tool 400) may be coupled to a motorized drive unit at the proximal end of the catheter system.

In certain implementations, an aspiration catheter such as a 16 French catheter is advanced transvascularly over a wire and/or through a larger diameter (e.g., 24 French aspiration catheter) to the treatment site. If the application of vacuum is not able to aspirate the clot into the 16 French catheter, an elongate flexible thrombus engagement tool may be advanced through the 16 French aspiration catheter, to facilitate retrieval of the clot.

Referring to FIGS. 16A and 16B, the thrombus engagement tool 400 may comprise an elongate flexible shaft 402 having a proximal end 404 and a distal end 406. A proximal hand piece such as a handle 408 may be configured to be rotated by hand. Distal end 406 carries a clot engagement tip 410 which may include one or more radially outwardly extending structures such as a helical thread 412. The handle 408 may have an indicium of rotational direction such as a printed or molded arrow 109 which indicates the direction to rotate the handle 408 in order for the helical thread 412 to engage clot.

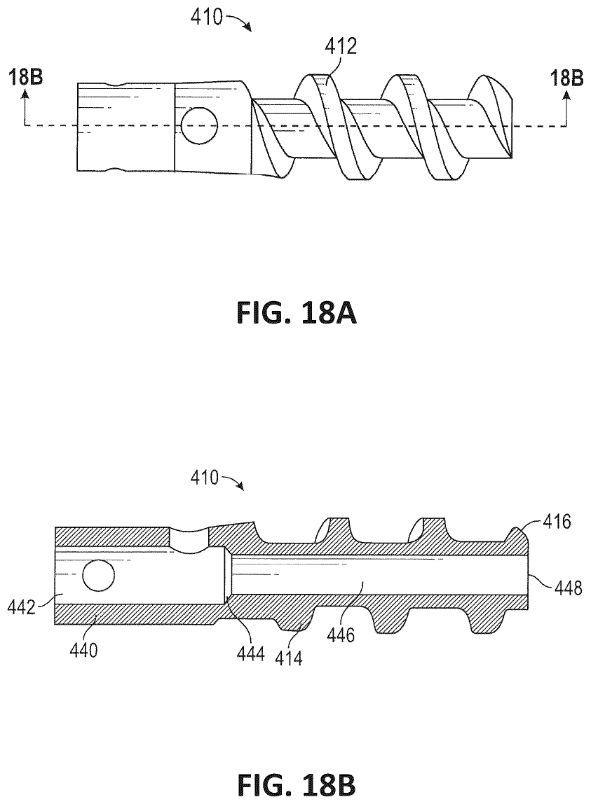

In one implementation illustrated in FIG. 16B, the thrombus engagement tool 400 carries a clot engagement tip 410 of the type illustrated in FIGS. 18A and 18B. The proximal end of the tip 410 is glued to the distal end of a braid-reinforced polyimide tube. The proximal end of the Microlumen has a cannulated torquing handle 408, and the whole assembly is cannulated so it can be delivered and function over a wire 468 such as an 0.035″ wire. The 0.035″ wire helps maintain space between the tip and the vessel wall, and the wire can be pulled back inside the working length of the flexible shaft 402 during rotation and engagement with the clot as needed.

Referring to FIG. 17A, the distal tip 410 includes a helical thread 412 extending from a distal end 414 to a proximal end 416 and supported by flexible shaft 402. The axial length of the distal tip 410 is at least about 2 mm or 5 mm or 10 mm and in some embodiments no more than about 30 mm or 20 mm measured along the flexible shaft 402. The helical thread 412 wraps around the axis at least about 1 or 2 or 4 or more full revolutions, but in some embodiments no more than about 10 or 6 revolutions. In some embodiments the axial length along the threaded portion of the tip is within the range of from about 1 to about 8 revolutions.

The helical thread 412 on this implementation may have a constant pitch throughout its length. The pitch may be within the range of from about 10 to about 20 threads per inch, or about 5 to about 10 threads per inch depending upon desired performance. Alternatively, the thread may have multiple pitches designed to engage, transport and grasp thrombus within the catheter lumen. A distal pitch may be less than a proximal pitch. The pitch may vary continuously along the length of the thread, or may step from a first, constant pitch in a proximal zone to a second, different pitch in a distal zone of the thread. The thread 412 may comprise a continuous single helical flange, or may have a plurality of discontinuities to produce a plurality of teeth or serrations, arranged helically around the core wire.

The side elevational profile or envelope scribed by the distal tip as it rotates may have a linear or nonlinear taper on one or both ends (e.g., football shaped) which provide varying diameter and thus clearance along its length from the generally cylindrical ID of the catheter lumen.

The maximum OD of the thread 412 is preferably smaller than the diameter of a sliding fit within the catheter lumen, and may generally be at least about 0.015 inches or 0.010 inches smaller than the catheter lumen ID. In some implementations, the Max OD of the tip may be significantly less than the inside diameter of the catheter lumen to allow more space for the thrombus, but still create significant grasping force via engagement of the helical threads with the thrombus. In one implementation, the maximum helical thread diameter is about 0.110 inches and the catheter lumen ID is about 0.275 inches (24 F) (a 0.165 inch gap between the helical threads and catheter wall.

In certain applications, the Max OD of the tip is no more than about 35% or no more than about 40% or no more than about 60% of the ID of the catheter, to leave a substantial tip bypass flow path. Since this implementation does not have any centering structures for the tip 410 or shaft 402, the tip will normally be pushed to one side of the aspiration lumen. When a clot becomes lodged between the tip and the opposing wall of the catheter, manual rotation of the tip can engage the clot like a worm gear and either grasp the clot (e.g., by pinning it against the opposing catheter sidewall) for retraction or facilitate freeing the blockage and aid in ingestion of the clot into the catheter.

The profile of the tip 410 viewed along the axis of rotation may be circular, or may vary to create a non circular pattern around the axis of rotation. The tip as seen in an end elevational view thus exhibits a major diameter and a minor diameter. The minor diameter may be no more than about 95% or 90% or 80% or 70% of the major diameter, depending upon desired performance.

Referring to FIGS. 17A and 17B, the illustrated tip 410 includes a distal advance segment 418 extending between an atraumatic distal tip at 420 and a transition to the distal end 416 of the thread 412. Helical thread 412 extends proximally from the transition to a proximal end 414 of the helical thread 412. A trailing segment 422 extends between the proximal end 414 of the thread and the proximal end 424 of the tip.

The axial length of the advance segment 418 may be at least about 1 cm or 2 cm and in some implementations is within the range of from about 2 cm to about 4 cm. The axial length of the helical thread 412 along the longitudinal axis is typically within the range of from about 1 cm to about 5 cm and in certain implementations between about 2 cm and 3 cm.

The outside diameter of the advance segment 418 at distal tip 420 is generally less than about 0.024 inches, or less than about 0.020 inches and, in one implementation, is about 0.018 inches. The maximum outside diameter of the advance segment 418 and helical thread 412 may be within the range from about 0.020 to about 0.045 inches, and, in one implementation, is less than about 0.040 inches, such as about 0.035 inches. The advance segment, helical thread and trailing segment of the tip 410 may be molded over the flexible shaft 402 using any of a variety of polymers known in the catheter arts.

Referring to FIG. 17B, a first radiopaque marker 430 may be carried on the flexible shaft 402 beneath the advance segment 418. A second radiopaque marker 432 may be carried on the flexible shaft 402 within the trailing segment 422. Each radiopaque marker may comprise a radiopaque tube or a coil of radiopaque wire such as a platinum iridium alloy wire having a diameter about 0.002 inches, and wrapped around the flexible shaft 402 and soldered to the flexible shaft 402 to produce an RO coil having an outside coil diameter of less than about 0.020 inches, such as about 0.012 inches. The radiopaque markers may also function as an axial interference fit between the flexible shaft 402 and the molded advance segment 418 and trailing segment 422 to resist core wire pull out from the tip 410.

In one implementation, the maximum OD of the thread 412 exceeds the maximum OD of the advance segment 418 by at least about 15% or 25% or 30% or more of the OD of the advance segment 418, to facilitate crossing the clot with the advance segment 418 and engaging the clot with the thread 412. The thread pitch may be within the range of from about 0.75 to about 0.30, or within the range of from about 0.10 and about 0.20, such as about 0.14 inches.

Preferably, the maximum OD of the tip 410 is less than about 60% or less than about 40% of the aspiration catheter ID at the distal end of the catheter, and may be within the range of from about 35% to about 55% of the catheter ID. In certain implementations, the maximum OD of the tip 410 may be within the range of from about 0.044 inches to about 0.041 inches within a catheter having a distal end ID within the range from about 0.068 inches to about 0.073 inches.

Depending upon the clinical application, it may be desirable to control the extent to which, if any, the distal tip 410 can extend beyond the distal end of the catheter. For example, distal extension of the distal end of the helical tip beyond the distal end of the catheter may be limited in some implementations to no more than about 5 mm or 3 mm or 1.5 mm or 1.0 mm or less. In other clinical environments the distal tip 420 may be permitted to extend at least about 2 cm or 3 cm and preferably as much as 4 to 8 cm beyond the catheter, but generally will be limited to extend no more than a preset distance such as 12 cm or 8 cm or 5 cm beyond the catheter depending upon desired performance. In one implementation, distal advance of the tip 410 is limited so that the distal end is within 2 cm or within 1 cm or no more than 0.5 cm in either the distal or proximal direction from the distal end of the aspiration catheter.

Distal advance of the tip 420 may be limited by providing mechanical interference at the desired distal limit of travel. In one implementation, a distal stop surface 440 on the handle 408 provides an interference engagement with a complementary proximal surface carried by the aspiration catheter through which the thrombus engagement tool 400 is advanced. Alternatively, a distal engagement surface can be carried anywhere along the length of the thrombus engagement tool 400, for sliding engagement with a complementary proximally facing stop surface carried by the catheter. Additional details may be found in U.S. Patent Application Pub. No. 2021/0093336 A1 published Apr. 1, 2021, and entitled Embolic Retrieval Catheter, which is hereby expressly incorporated in its entirety herein by reference.

The limit on distal advance of the helical tip may include a first configuration in which distal advance is limited to a first position proximate the distal end of the evacuation catheter to prevent injury to the vascular wall. Upon a user initiated adjustment, the helical tip may be advanced to a second position farther out of the distal end of the catheter such as for inspection and cleaning purposes. This adjustment of the limiting mechanism may be locked out following cleaning or inspection, to limit distal travel to the first position to prevent an undesired degree of exposure of the helical tip element when the system is within the patient's vasculature. Any of a variety of movable interference levers of pins may be engaged to limit travel to the first position, or disengaged to allow travel to the second position.

Referring to FIGS. 18A and 18B, a tip 410 includes a tubular sidewall 440 defining a hub having a connector such as a cavity 442 for coaxially receiving the distal end of a support shaft such as a braid reinforced polyamide tube. The inside diameter of the cavity 442 steps down at a distal end of the hub at a step 444 to a smaller diameter lumen 446 in communication with a distal opening 448. This provides a continuous lumen throughout the length of the micro lumen shaft and tip 410 so that the thrombus engagement tool can be introduced over the wire.

In general, the pitch of thread 412 may be within the range of from about 0.07 to about 0.11, and in one embodiment, is about 0.09. The width of the thread 412 measured along an axis that is perpendicular to a face of the thread may be within the range of from about 0.009 to about 0.04, and, in one embodiment, is about 0.02. The greatest major diameter of the thread 412 may be at least about 10%, or at least about 15%, or at least about 20% greater than the diameter of the proximal hub end of the tip 410 surrounding the cavity 442. In one implementation, the outside diameter of the proximal hub is about 0.090 inches and the outside diameter of the thread 412 is about 0.110 inches. The actual length of the tip 410 including the proximal hub may be within the range of from about 0.2 inches to about 0.8 inches and in some implementations within the range of from about 0.4 inches to about 0.6 inches.

The tip 410 may be manufactured in accordance with any of a variety of techniques known in the art, such as machining, etching, additive and/or subtractive processes. In one implementation, the tip 410 is molded from a polymer such as PEBAX, which may be a 55 D hardness. The PEBAX may include a radiopaque agent, such as bismuth sub carbonate, present in the range of from about 50% to about 70% by weight.

Any of the tip dimensions and configurations disclosed herein may be re-combined with any of the other tip dimensions, configurations, drive shafts and associated structures depending upon the desired clinical performance.

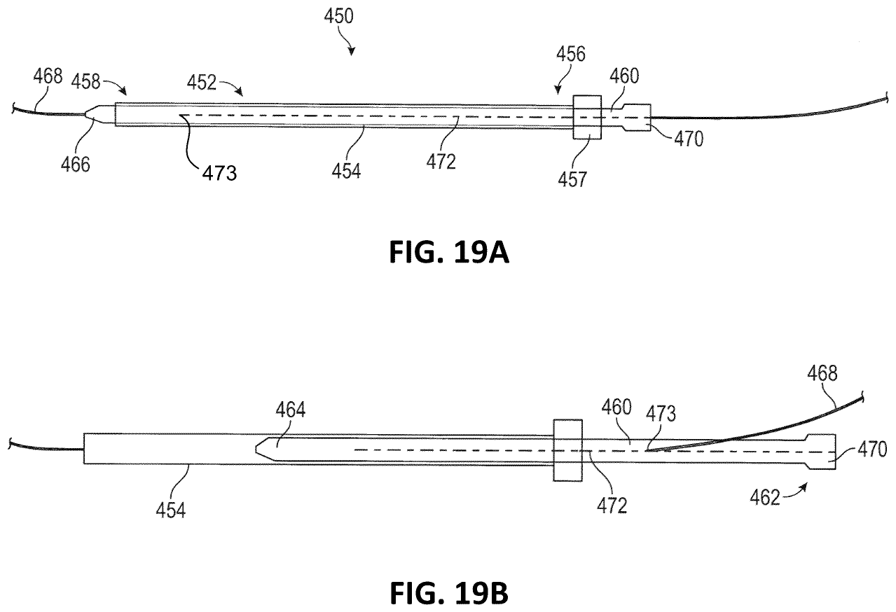

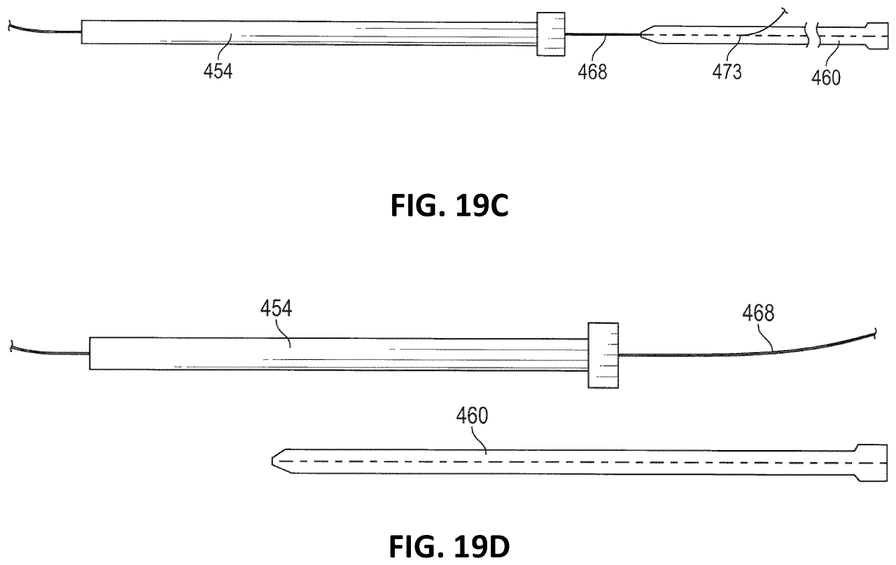

Referring to FIGS. 19A-19D, there is illustrated a split dilator system 450 which may be utilized with any of the catheters disclosed herein. The system includes a catheter 452 having an elongated tubular body 454 extending between a proximal end 456 and a distal end 458. Proximal end 456 is provided with a proximal hub or manifold 457 as has been discussed in connection with other catheters disclosed herein.

An elongate flexible dilator 460 has a length sufficient to extend throughout the entire length of the catheter 452. Dilator 460 extends between a proximal end 462 and a distal end 464 having a tapered distal tip 466. The dilator 460 is provided with a central lumen (not illustrated) so that it may be advanced over a guide wire 468. Proximal end 462 of the dilator is provided with a proximal hub 470.

A split 472 extends the length of the hub 470 and along the sidewall of the tubular dilator 460. The split may be in the form of a slot extending through the entire wall thickness of the dilator, a perforation line, a groove, or other weakening to allow the formation of a slit through the dilator side wall, and through which the guide wire 468 may be laterally removed as discussed further below. The longitudinal split 472 may extend the entire length of the dilator 460, or extend from the proximal end in a distal direction to an endpoint 473 within the range of from at least about 2 cm or 5 cm to no more than about 40 cm or 30 cm from the tapered tip 466.

Preferably, a first locking component carried by the hub 470 is releasably engageable with a complementary second locking component carried by the hub 457.

Referring to FIG. 19B, following trans vascular advance of the catheter and dilator assembly to the desired intravascular location, the dilator 460 may be proximally removed leaving the catheter 452 in place. Desirably, the guide wire 468 may remain unmoved in position at the target vascular site while removing the dilator 460, preferably without the need for a proximal guide wire extension. For this purpose, the guide wire 460 may be laterally progressively removed from the dilator at a parting point 473 that advances axially along the split 472, as the dilator 460 is proximally retracted from the catheter 452 and guidewire 468.

Once the tapered tip 466 has been proximally retracted from the catheter, the guide wire 468 may be grasped between the dilator 460 and the catheter 462, and the dilator 460 may be proximally removed from the catheter 452 and from the guide wire 468. This allows removal of the dilator without disturbing the position of the catheter or the guide wire, which are thereafter available for a subsequent intravascular procedure.

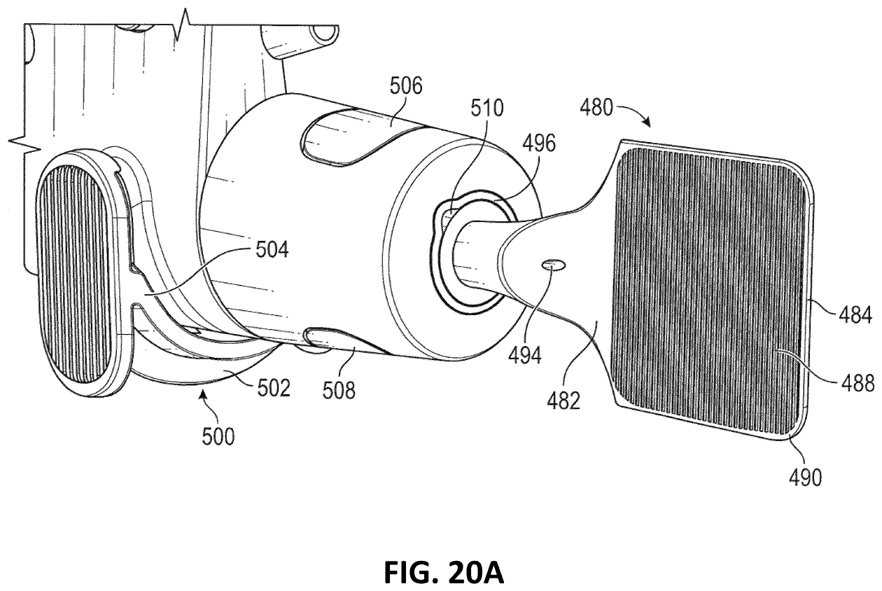

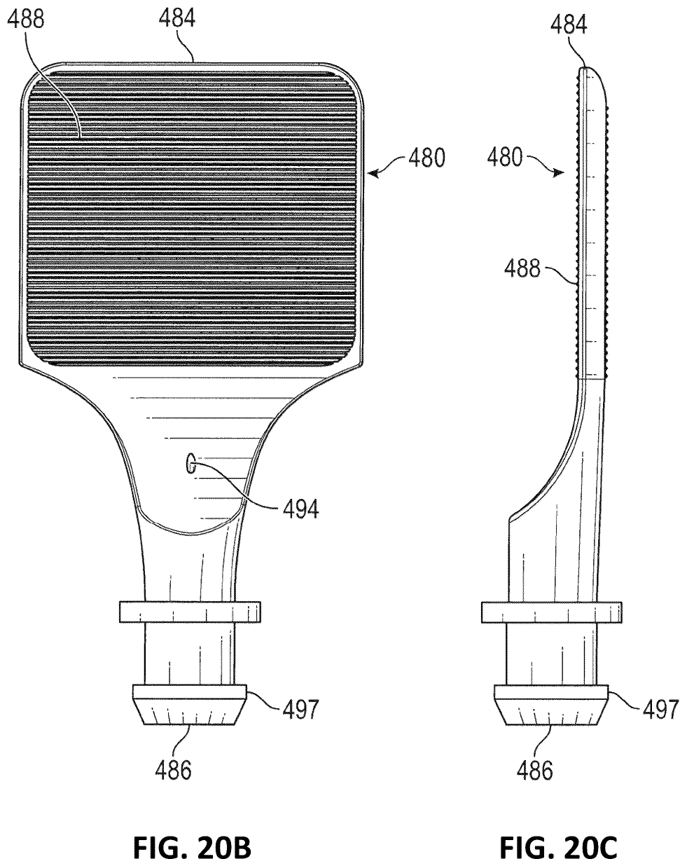

Referring to FIGS. 20A and 20B, there is illustrated a proximal dilator handle 480. The handle 480 comprises a body 482 having a proximal end 484 a distal end 486 and a longitudinal axis. At least a first proximal gripping surface 488 is carried by the body. In the illustrated implementation, a first gripping surface 488 is provided on at least one side of a paddle shaped grip 490, configured to be held between a thumb and forefinger. A second gripping surface 492 may be provided on an opposing side of the handle. Gripping surfaces may be provided with a friction enhancing surface structures such as a plurality of ridges oriented transverse to the longitudinal axis of the dilator handle 480.

A proximal exit port 494 in communication with the dilator guidewire lumen is oriented along the longitudinal axis of the dilator handle 480, such that a guide wire extending out of the exit port 494 lies along the first gripping surface 488. This allows a clinician to pin the guide wire to the gripping surface 488 using a finger such as a thumb, thereby enabling the dilator and the guide wire to be moved as a unit using one hand.

The dilator may be removably secured to the catheter such as by a retention clip 496 carried by the proximal end of the handle. A release such as a button or deformable interference snap fit may be provided to unlock the dilator handle from the housing, enabling the dilator to be proximally withdrawn from the catheter. In the illustrated implementation, a retention surface such as a proximal surface of a retention ring 497 carried by proximal end 486 of the body 482 provides an interference fit with the retention clip 496. This combines the dilator and handle/catheter into a single system. The paddle may be released from the retention clip by depressing at least a first button 506 and as illustrated also a second button 508 carried on the upper and lower sides of the retention clip housing, and proximally withdrawing the paddle.

This is the same connection and release dock for use with a thrombus engagement tool such as engagement tool 400 discussed in connection with FIGS. 16A and 16B. A distal limit safety feature on the thrombus engagement tool 400 fits into the retention clip 496, ensuring that the distal tip of the tool 400 can not be advanced forward beyond the distal tip of the catheter without both aligning a projection on the tool 400 with the rotational key 502 and intentionally advancing the tool 400 through the retention clip while depressing at least the first button 506 or other unlock control.

Once the distal limit has been released, the tip 410 may be distally advanced no more than about 4 cm and generally about 1 cm to 2 cm beyond the distal end of the catheter. This is intended to be accomplished once the thrombus engagement tool has been withdrawn from the patient, to allow visual inspection of the tip 410.

The engagement tool 400 may also be proximally retracted within the catheter, typically for less than about 3 cm or less than about 2 cm, and may be provided with a spring bias to return to approximate axial alignment between the distal end of the tip 410 and the distal end of the catheter.

A hemostasis clamp 500 may be provided, to hold the hemostasis valve open such as during shipping, or during the advance or withdrawal of devices therethrough. The hemostasis valve is opened by depressing at least a first control button, and in the illustrated implementation first and second control buttons positioned on opposing sides of the handle. The hemostasis clamp comprises a generally U shaped body 502 having a first arm 504 configured to depress a first button, and a second opposing arm (not illustrated) configured to depress a second button on an opposite side of the handle. The hemostasis clamp 500 may be removably retained on the handle by a friction fit, or an interference fit between the handle and the body which can be overcome by plastic deformation as the body is pulled away from the handle to release the hemostasis control buttons.

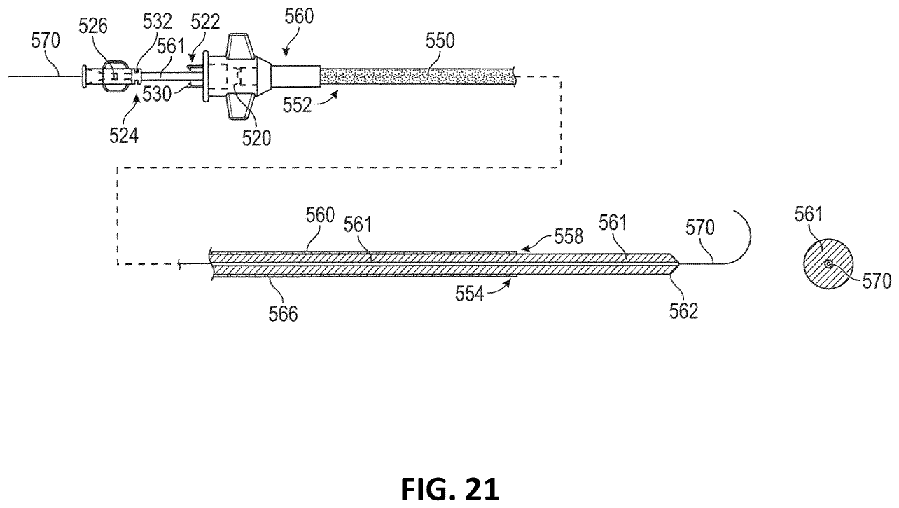

Referring to FIG. 21, an elongate flexible cannulated rail or dilator 561 is shown extending over the guidewire 570 and occupying the space between the guidewire 570 and the large inside diameter of the central lumen 558 of the large diameter catheter 560 to provide support to the catheter and/or an atraumatic tip during delivery.

This catheter-cannulated rail-guidewire assembly is intended to easily track through anatomical challenges more easily than the catheter. The catheter-rail-guidewire assembly then acts as a first stage of the catheter delivery system and enables the large diameter catheter or catheter system to be inserted and independently advanced over this first stage into a blood vessel (e.g. the femoral vein) percutaneously over a guidewire and advanced through potentially tortuous vasculature to the remote target location of interest without requiring advanced skills or causing kinking of the catheter.

The cannulated rail 561 may comprise a soft flexible cylindrical body having a guidewire lumen with a diameter of no more than about 0.040″ and an outside diameter no less than about 0.025″ or about 0.010″ smaller than the inner diameter of the large diameter catheter. Thus the wall thickness of the cannulated rail 561 is typically at least about 0.010″ less than the radius of the large diameter catheter and in some implementations at least about 0.120″ or more, depending upon the size of the annular space between the inside diameter of the catheter and the outside diameter of the guidewire.

The cannulated rail 561 may have an elongated tapered distal tip 562 that may project beyond the distal end 554 of the catheter 560. The thick sidewall of the cannulated rail 561 may comprise one or more flexible polymers, and may have one or more embedded column strength enhancing features such as axially extending wires, metal or polymeric woven or braided sleeve or a metal tube, depending upon the desired pushability and tracking performance along the length of the dilator.

Optionally, the proximal segment of the rail or dilator which is not intended to extend out of the distal end of the catheter may be a structure which is not coaxial with the guidewire, but a control wire which extends alongside the guidewire in the catheter and allows the distal tubular telescoping segment of the rail or dilator to be retracted or extended. (analogous to rapid exchange catheters) without the entire length of the rail structure being over the wire. This allows removal or insertion of the rail or dilator over a shorter guidewire because of the shorter coaxial segment tracking over the guidewire.

Catheter 560 may be provided with a proximal hub 520, having a port for axially movably receiving the rail 561 therethrough. The hub 520 may be provided with an engagement structure such as a first connector 522 for releasably engaging a second complementary connector 524 on a hub 526 on the proximal end of the rail 561. First connector 522 may comprise an interference structure such as at least one radially moveable projection 530, for releasably engaging a complementary engagement structure such as a recess 532 (e.g., an annular ridge or groove) on the hub 526. Distal advance of the rail 561 into the catheter 560 causes the projection 530 to snap fit into the recess 532, axially locking the catheter 560 and rail 561 together so that they may be manipulated as a unit.

The dilator is inserted through the hemostasis valve in the hub 520 of a large bore (e.g., 24 F) catheter 560 and advanced through the catheter until the retention clip on the dilator hub 526 or catheter hub 520 snaps into the complementary recess on the other hub. In this engaged configuration, an advance segment along the flexible distal end of the 24 Fr rail dilator 561 will extend at least about 5 cm or 10 cm, and in some implementations at least about 15 cm or 20 cm beyond the distal end 554 of the 24 Fr catheter 560. The rail dilator and 24 Fr catheter system are thereafter distally advanced over a previously placed guidewire and into the introducer sheath.

The dilator and catheter combination differentiate over prior systems both because of the flexibility of a distal zone of the dilator and greater length of the dilator than the corresponding catheter. Typically, a dilator is a uniform stiffness and length-matched to its catheter, with only a short atraumatic tip of the dilator extending beyond the distal end of the catheter. The dilator has a supportive proximal end and a flexible distal end, with a total dilator length much longer than the catheter 60 to enable, as an example, the following procedure.

In use, a guidewire 570 such as an 0.035″ guidewire is advanced under fluoroscopy using conventional techniques into a selected vessel. The cannulated rail 561, optionally with the catheter 560 mounted thereon, is loaded over the proximal end of the guidewire 570 and advanced distally over the wire until the distal end of the rail is in position at the target site.

The 24 Fr catheter 560 is thereafter unlocked from the rail 561 and advanced over the rail 561 to the desired site, supported by the rail 561 and guidewire 570 combination. Because the uncovered advance section of the rail has already traversed the challenging tortuosity through the heart, the catheter 561 now just slides over the advance section of the rail for easy passage to the final target location. The supportive proximal zone and flexible distal advance section of the rail enables ease of delivery through the most challenging anatomy in, for example, a PE procedure going from the vena cava through the tricuspid and pulmonary valves of the heart into the central pulmonary artery without concern about damaging the tissue (atraumatic, flexible tip) or damaging the dilator (high kink resistance due to flexible, high wall thickness “solid” dilator construction.

The cannulated rail 561, or the cannulated rail 561 and the guidewire 570 combination, may thereafter be proximally withdrawn, leaving the large bore catheter 560 in position to direct a procedure catheter such as any of the aspiration catheters disclosed elsewhere herein to the target site.

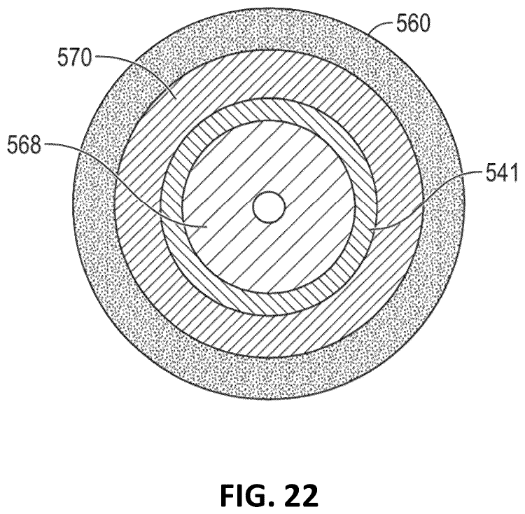

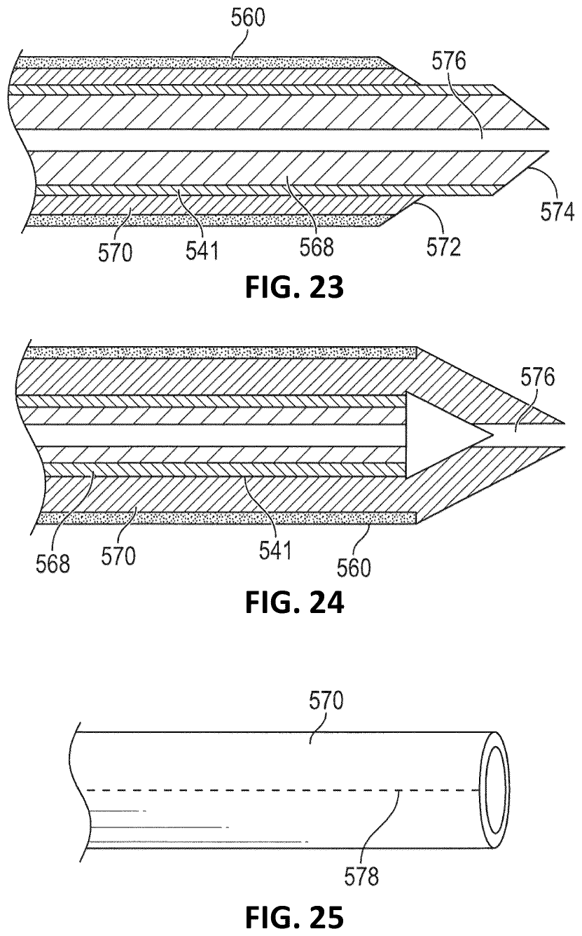

Referring to FIG. 22, the large diameter (LD) catheter 560 may in some situations have a smaller diameter (SD) catheter though its central lumen for the purposes of introducing an additional functionality (e.g., clot grabber catheter 562, imaging catheter 10, or mechanical thrombectomy tool 66) and/or telescoping the SD catheter to more distal locations in the anatomy. In order to enable delivery of the LD catheter 560 and SD catheter as a single system, the SD catheter may have a core dilator 568 for support, and the gap between the outer diameter of the SD catheter and inner diameter of the LD catheter 560 may be maintained or supported by a second, tubular dilator 571. The tubular dilator 571 may have a shaped distal tip 572 for a smooth tapered transition from the SD catheter 541 to the LD catheter 540. The distal end 534 of the core dilator may be provided with a complementary taper to the distal taper of the thin wall SD dilator (FIG. 23) or may end at the distal end of the LD catheter (FIG. 24).

The core dilator 568 inside the SD catheter 541 and tubular dilator 570 between the two catheters may have an interlocking feature to create a single (SD+LD) catheter+(core+tubular) dilator system. For example, complementary connectors may be provided on hubs on the proximal ends of the system components.

Referring to FIG. 24, the tip of the tubular dilator 570 may be configured to taper to the guidewire lumen 576, thus covering and extending distally beyond the small diameter catheter 541 if it is in place. The tip of the tubular dilator 570 may be provided with a longitudinally extending slit 578, scored or perforated one or more times to allow the tip to split longitudinally and be pulled back into the space between the LD and SD catheters and fully expose the distal end of the small diameter catheter 541. See FIG. 25.

The single (SD+LD) catheter+(core+tubular) dilator system may be pre-assembled and detachably interlocked at the proximal hub. Additional tubular dilators having a series of outside diameters and wall thicknesses may be provided such that the SD catheter may be used in combination with different diameter LD catheters. A LD catheter may be used with different SD catheters by providing tubular dilators having the same OD but a series of different inside diameters. The core+tubular dilators may simply be pulled proximally to withdraw both dilators as a single system, or the tubular dilator may be configured with a tab or handle at the proximal end and a slit, scoring, perforation or other mechanism so as to split, peel, or tear it along the longitudinal axis during withdrawal to allow the tubular dilator to peel from the SD catheter as it slides proximally out of the space between the LD and SD catheters. (FIG. 25).

FIG. 26 shows an embodiment of a thrombectomy catheter 600 (which may also be referred to herein as an aspiration catheter) that may be utilized in a vacuum aspiration system as described previously herein or as further described below. The thrombectomy catheter 600 may be used in the same or similar manner, and incorporate the same or similar components or features, as the other thrombectomy or aspiration catheters described above, such as the larger catheters (e.g., 24 French catheters), and may include additional components or features as described further herein.

The thrombectomy catheter 600 can include a handle 606, also referred to herein as a housing, having a proximal end 606a, a distal end 606b opposite the proximal end 606a, and an elongate catheter body 608 extending distally from the distal end 606b. A proximal end 608a of the elongate catheter body 608 can be permanently carried by the handle 606 or may be provided with a releasable connector for detachable connection to a complementary connector on the handle 606. The distal end 608b of the catheter body 608 provides a distal opening to provide aspiration for clot (e.g., thrombus and/or embolus) removal and is illustrated as having a flat or straight end perpendicular to the longitudinal axis of the catheter body 608. In other embodiments, distal end 608b may have an inclined tip to increase an area of the aspiration opening. Near the distal end 608b, a radiopaque marker (not shown) may be provided to assist in visualization. The catheter body 608 may be flexible to provide for transvascular and trackability. In some embodiments, the sidewall of the catheter body comprises one or more sections including a helical coil, such as a helical coil extending to the distal end 608b or to proximal of a radiopaque marker (not shown) provided at the distal end.

A central lumen extending through the elongate catheter body 608 can be in communication with a flow path extending through the handle 606 to a proximal access port 612 positioned on a proximal end 606 of the handle 606. The flow path between the elongate catheter body 608 and the proximal access port 612 can be linear, to axially movably receive a second catheter, such as the second catheters described above or further herein, which may or may not be utilized in a given procedure. To accommodate the absence of a second catheter and seal the access port 612, the handle 606 can include a hemostasis valve actuator 614 for opening and/or closing a hemostasis valve inside the handle 606. The hemostasis valve can include a Tuohy-Borst valve. Further details regarding embodiments of a flow path and internal components within the handle 606, which may be applied to any of the catheters described herein, are described with respect to FIGS. 15A and 15B above and elsewhere within this specification.

The handle 606 can include a control such as lever 616 which can be similar or identical to the lever 117, which is described in relation to FIG. 15B. Although reference is made to the control including a lever 616, the control can include a button, a slide switch, a toggle switch, or any other suitable mechanism. In some cases, the lever 616 can be spring actuated and/or include a locking feature to maintain the lever 616 in a constant position. The lever 616 can turn aspiration on and off by controlling a two-way or a three-way manifold valve 642, which can be similar or identical to valve 200, for selectively controlling fluid flow. The valve 642 can include a flow path 642a extending therethrough. In some cases, the lever 616 can be actuated between at least two positions. The lever 616 as illustrated may be rotated in a forward or distal direction to move into the “On” position. In this first position, as shown in FIG. 27A, the flow path 642a of the valve 642 can place the clot container 620 and the catheter body 608 in communication with each other via a conduit 671 having a lumen 672 therein. When the lever 616 is in the first position, the catheter body 608 may be in fluid communication with a source of vacuum via the clot container 620, which is described further herein and is similar to filter 206 described above, and the aspiration tubing 618. The fluid flow path along the aspiration tubing 618, the clot container 620, and the elongate catheter body 608 can supply vacuum at a distal end of the elongate catheter body 608, which can facilitate removal of clots from the vasculature of a patient. In some cases, the aspiration tubing 618 can be in fluid communication with the clot container 620 via a port 683. When the lever 616 is in the first position, vacuum from the source of vacuum can be supplied to the distal end of the elongate catheter body 608 via a path including the aspiration tubing 618, the port 683, the clot container 620, the flow path 642a, the lumen 672, and the elongate catheter body 608.

In the second position (e.g., an “Off” position), the lever 616 can cause the valve 642 to place the elongate catheter body 608 in fluid communication with a multiport 619. For instance, as illustrated in FIG. 27B, the flow path 642a of the valve 642 can place the elongate catheter body 608 and the multiport 619 in communication with each via the lumen 672. The lever 616 as illustrated may be rotated in a rearward or proximal direction to move into the “Off” position. This can beneficially allow delivery of medications, contrast media, and/or saline to the patient. In some cases, fluids, medications, contract media, and/or saline can be injected to the multiport 619 for delivery to a patient using, for example, a syringe. When the lever 616 is in the second position, the valve may prevent communication between the elongate catheter body 608 and the aspiration tubing 618 thereby preventing aspiration at the distal end of the elongate catheter body 608. In some cases, the multiport 619 can extend inside the handle 606 and be in fluid communication with the valve 642, the flow path 642a, the lumen 672, and the elongated catheter body 608, at least when the lever 616 is in the second position. A distal end of the multiport 619 can be in fluid communication with the valve 642 via a port 682, one end of the lumen 672 can be in fluid communication with the valve 642, and another end of the lumen 672 can be in fluid communication with the elongate catheter body 608. In some embodiments, the valve 642 can fluidly connect the lumen 672 with the multiport 619 when the lever 616 is in the second position.

The handle 606 can include a clot container 620 (also referred to herein as a filter assembly). The clot container may be similar to and incorporate features of filter 206 described above, and vice versa. The clot container 620 can include a housing 622, at least a portion of which may include a transparent window 626. The window 626 can allow visualization of the contents (e.g., aspirated clot, liquid) inside a filter chamber 628, which can include a filter 641. As shown in FIGS. 27A and 27B, the clot container can be positioned on a distal side of the handle, distal to the valve 642, with the valve located fluidically between the catheter body and the filter or filter chamber. In some cases, the filter 641 can include a mesh opening of about 200 microns (m). The filter 641 can include a mesh opening from about 100 μm to about 300 μm, from about 120 μm to about 280 μm, from about 140 μm to about 260 μm, from about 160 μm to about 240 μm, from about 180 μm to about 220 μm, from about 190 μm to about 210 μm, and/or from about 195 μm to about 205 μm.

The clot container 620 can place the filter in the flow path between the elongate catheter body 608 and an aspiration tubing 618. The clot container 620 can be removably attached to the handle 606. When the clot container 620 is attached to the handle 606, the filter chamber 628 can be closed (e.g., sealed) to maintain negative pressure conveyed from a pump via aspiration tubing 618. The clot container 620 can be detached from the handle 606 to permit insertion, removal, and/or replacement of the filter and/or to remove any clots or liquid that has accumulated in the clot container.

The handle 606 can include a vent, which may be in the form of a vent button 632. The vent button 632 can be similar or identical to the vent 117 which is described in relation to FIGS. 9, 10A, and 11A. As illustrated in FIGS. 26, 27A, and 27B, the vent button 632 may be positioned on a proximal side of the handle 606, proximal to the valve 642. The vent button 632 can be in fluid communication with a vent (aperture) to atmosphere or a vent fluid source and the filter chamber 628. For instance, the vent button may be in fluid communication with the space or chamber 628 of the clot container 620, or along a flow path between the filter chamber 628 and the elongate catheter body 608. Actuation of the vent button 632 can permit a flow of air to enter the clot container 620, which may allow the filter chamber 628 to be at least partially evacuated through the aspiration tubing. Evacuating the clot container can also increase visibility of a clot inside the clot container 620 by removing the blood surrounding the clot. Opening the vent can also reduce vacuum pressure in the clot container 620 to facilitate removal of the clot container 620 from the handle 606 and/or reduce vacuum pressure at the distal end 608b of the catheter body 608 to facilitate agitation of a clot (e.g., using a thrombus engagement tool). The vent button 632 and/or the vent (aperture) to atmosphere may be in fluid communication with the clot container 620 regardless of the position of the valve 642 (e.g., the position shown in FIG. 27A and the position shown in FIG. 27B)—i.e., regardless of whether the valve is open or closed. This can allow for the evacuation of the clot container 620 regardless of whether vacuum is being supplied to the elongate catheter body 608. For instance, the vent button 632 may allow the filter chamber 628 to be at least partially evacuated even when vacuum is not being supplied to the elongate catheter body 608 (e.g., when the lever 616 is in the “off” or second position).

In some cases, the elongate catheter body 608 can include a flexible neck 609 extending between a proximal end 608a and a transition 611. The flexible neck 609 can be similar or identical to the flexible neck 109, which is described in relation to FIG. 10A. The flexible neck 609 can have a greater flexibility than the adjacent portion of the elongate catheter body 608 distal to the transition 611. The flexible neck 609 may have a length of at least about 2 cm and often at least about 4 cm, but generally no more than about 20 cm or 10 cm or less.

The proximal end 608a of the elongate catheter body 608 can be provided with a strain relief (not shown) and a rotation control such as a rotatable knob assembly. The rotatable knob assembly can include a rotatable knob 615 and a rotation sleeve (not shown). The rotatable knob 615 may be rotationally fixed to the elongate catheter body 608 and rotatable with respect to the handle 606. This facilitates relative rotation between the elongate catheter body 608 and the handle 606. For example, to adjust the position of the handle 606 relative to the elongate catheter body 608 without disturbing the position of the elongate catheter body 608 inside the vasculature of a patient, the rotatable knob 615 can be held in place and the handle 606 can be rotated relative to the rotatable knob 615. This can beneficially allow users to adjust the position of the handle 606 while preventing movement of the elongate catheter body 608. The rotatable knob 615 and the catheter body 608 can include a friction fit allowing the rotatable knob 615 to rotate the catheter body 608 when the rotatable knob 615 is rotated. In some cases, rotation of the handle 606 can cause simultaneous rotation of the handle 606 and the elongate catheter body 608.

In some cases, as illustrated in FIG. 27C, an aspiration system 900 can include two or more thrombectomy catheters. For example, the aspiration system 900 can include a first thrombectomy catheter 902, which can be similar or identical to the thrombectomy catheter 600, and a second thrombectomy catheter 904. In some cases, the thrombectomy catheter 904 can be smaller than the first thrombectomy catheter 902 to allow the first thrombectomy catheter 902 to receive the second thrombectomy catheter 904. The second aspiration catheter 904 can be similar or identical to the second aspiration catheter 104, which is described in relation to FIG. 9. In some cases, the second thrombectomy catheter 904 can be advanceable through the first thrombectomy catheter 902. The first and second thrombectomy catheters 902, 904 can be used independent of each other. In some cases, the second thrombectomy catheter 904 can include a handle 940 including a proximal end 940a and a distal end 904b. The handle 940 can be similar or identical to the handle of the first thrombectomy catheter 902. An elongate flexible catheter 958 can extend distally from the distal end 904b of the handle 940 and can be advanced distally through the first thrombectomy catheter 902 and the elongate flexible catheter 908 of the first thrombectomy catheter 902. In some cases, at least a portion of the elongate flexible catheter 958 of the second thrombectomy catheter 904 can have an outer diameter smaller than an inner diameter of the elongate flexible catheter 908 to allow the elongate flexible catheter 958 to extend through the elongate flexible catheter 908. For example, the elongate flexible catheter 908 can have a diameter of or about 24 Fr and the elongate flexible catheter 958 can have a diameter of or about 16 Fr.

In some cases, a distal end of the elongate flexible catheter 958 can be curved (e.g., pre-bent) or be provided with an active deflection mechanism such as a slotted sidewall and an axially extending pull wire. The distal end of the elongate flexible catheter 958 can maintain a straight configuration when the elongate flexible catheter 958 is being delivered into the vasculature of patient. The elongate flexible catheter 958 can be delivered into the vasculature using a dilator. When the dilator is removed, the distal end of the elongate flexible catheter 958 can retake the pre-shaped bent or curve. This can beneficially direct the distal end of the elongate flexible catheter 958 toward a clot.

The first and second thrombectomy catheters 902, 904 can be in fluid communication with a pump assembly 950 via aspiration tubing 918, 968, 969, a valve 970, and aspiration line 911. In some cases, an aspiration canister 913 can positioned along the aspiration line 911. The pump assembly 950 can include an aspiration pump 951 which may have a vacuum pump. The aspiration pump 951 can be similar or identical to the aspiration pump 50, which is described in relation to FIGS. 1-5.

The valve 970 can be actuated between at least two positions to allow and/or restrict fluid flow between the pump assembly 950 and the first and second thrombectomy catheters 902, 904. For example, in a first position, the valve 970 can place the first thrombectomy catheter 902 in fluid communication with the pump assembly 950 but restrict fluid communication between the second thrombectomy catheter 904 and the pump assembly 950. In a second position, the valve 970 can place the second thrombectomy catheter 904 in fluid communication with the pump assembly 950 but restrict fluid communication between the first thrombectomy catheter 902 and the pump assembly 950. In a third position, the valve 970 can place the first and second thrombectomy catheters 902, 904 in fluid communication with the pump assembly 950. In a fourth position, the valve 970 can restrict fluid communication between the first and second thrombectomy catheters 902, 904 and the pump assembly 950.

In some cases, a proximal access port of the second thrombectomy catheter 904 can receive a thrombus engagement tool which may be the same or similar as the thrombus engagement tool described above, but with a larger outer diameter and/or a greater length. For example, a proximal access port 962 of the second thrombectomy catheter 904 can receive a thrombus engagement tool similar or identical to the thrombus engagement tool 400, which is described in relation to FIGS. 16A and 16B. The diameter of the thrombus engagement tool can include an outer diameter smaller than the thrombus engagement tool 400 to allow the thrombus engagement tool to be advanced through the second thrombectomy catheter 904. In some cases, the aspiration system 900 can include two thrombus engagement tools 400. In some embodiments, a first thrombus engagement tool can include an outer diameter of or about 24 Fr and a second thrombus engagement tool can include an outer diameter of or about 16 Fr. Although reference is made to thrombus engagement tool with these two outer diameters, the thrombus engagement tool 400 can include a diameter smaller than or larger than 16 Fr and/or 24 F.

An outer diameter of the helical threads on the first and/or second thrombus removal tools can be smaller than an inner diameter of the elongate flexible catheter 908 and/or the elongate flexible catheter 958, so there is a space between the helical thread and the interior of the elongate flexible catheter 908 and/or the elongate flexible catheter 958. This can beneficially allow the first and/or second thrombus removal tools to engage a clot between the helical thread and the interior of the elongate flexible catheter 908 and/or the elongate flexible catheter 958. The interior diameters of the elongate flexible catheter 908 and/or the elongate flexible catheter 958 can be constant throughout an entire length of the elongate flexible catheter 908 and/or the elongate flexible catheter 958. This can beneficially prevent or reduce restrictions on clot translation along the lumen of the elongate flexible catheter 908 and/or the elongate flexible catheter 958.

The elongate flexible shaft 402 of the thrombus engagement tool 400 can extend distally from the proximal end 940a of the handle 940 and is configured to advance distally through the first thrombectomy catheter 902 and the elongate flexible catheter 908. The elongate flexible shaft 402 can extend to a distal end of the elongate flexible catheter 958. In some cases, the handle 408 can be removably secured to a retention clip 996 positioned on the handle 940. This can prevent the distal end of the elongate flexible shaft 402 from extending beyond a distal end of the elongate flexible catheter body 958 when the handle 408 is secured to the retention clip 996.

### EXAMPLE EMBODIMENTS

The following are example embodiments that may be utilized or combined with other embodiments described further in this specification.

Embodiment 1: An aspiration system with accelerated response, comprising one or more of the following:
an aspiration pump in communication with a first chamber;
an aspiration catheter configured for placement into fluid communication with the first chamber by way of an aspiration tube;
a second chamber in between the aspiration tube and the catheter; and
a valve between the second chamber and the aspiration catheter;
wherein upon opening of the valve with negative pressure in the first and second chambers, resistance to fluid flow between the second chamber and the distal end of the catheter is less than the resistance to fluid flow between the second chamber and the first chamber, causing a rapid aspiration into the second chamber.

Embodiment 2: An aspiration system as described in any embodiment herein, further comprising a handle on the aspiration catheter, and the second chamber is carried by the handle.

Embodiment 3: An aspiration system as described in any embodiment herein, further comprising a first control on the handle for opening the valve.

Embodiment 4: An aspiration system as described in any embodiment herein, wherein the valve is normally closed and actuation of the control momentarily opens the valve.

Embodiment 5: An aspiration system as described in any embodiment herein, further comprising a second control for activating the pump.

Embodiment 6: An aspiration system as described in any embodiment herein, further comprising a hemostasis valve carried by the handle.

Embodiment 7: An aspiration system as described in any embodiment herein, wherein the hemostasis valve comprises a collapsible tubular sidewall defining a valve lumen, and a filament formed into a loop around the tubular sidewall and configured to collapse the valve lumen.

Embodiment 8: An aspiration system as described in any embodiment herein, wherein the hemostasis valve further comprises a frame and a lever, and the filament has at least a first tail portion extending away from the loop, around a first fulcrum on the lever and is secured against axial movement with respect to the frame.

Embodiment 9: An aspiration system as described in any embodiment herein, wherein the first tail portion is connected to the frame.

Embodiment 10: An aspiration system as described in any embodiment herein, further comprising a second lever, and the filament further comprises a second tail portion extending from the loop, around a second fulcrum on the second lever and is connected to the frame.

Embodiment 11: An aspiration system as described in any embodiment herein, wherein the aspiration tube is at least about 50 inches long.

Embodiment 12: An aspiration system as described in any embodiment herein, wherein the second chamber is configured to capture clot aspirated by the catheter.

Embodiment 13: An aspiration system as described in any embodiment herein, wherein at least a portion of the second chamber is removably carried by the handle.

Embodiment 14: An aspiration system as described in any embodiment herein, wherein the second chamber comprises a filter membrane spaced apart from a transparent wall.

Embodiment 15: An aspiration system as described in any embodiment herein, comprising a tubular filter membrane, spaced radially inwardly apart from a transparent outer tubular wall.

Embodiment 16: An aspiration system as described in any embodiment herein, further comprising an operator actuated control, configured to toggle a flow regulator between a default low flow mode, and a momentary, operator initiated high flow override mode.

Embodiment 17: An aspiration system as described in any embodiment herein, wherein the second chamber is configured for location within a sterile field, and the first chamber is configured for location outside of the sterile field.

Embodiment 18: An aspiration system as described in any embodiment herein, further comprising a handle on the aspiration catheter, a tube between the handle and the second chamber, and the tube is no more than about 20 inches long.

Embodiment 19: A split dilator aspiration system, comprising one or more of the following:
a catheter, having an elongate, flexible tubular body with a proximal end, a distal end, a side wall defining a central lumen, and a handle on the proximal end; and
a dilator, advanceable through the central lumen, the dilator having an elongate body, cannulated to receive a guidewire, and an axially extending split along at least a portion of the elongate body, configured to allow removal of a portion of the dilator laterally from the guidewire.

Embodiment 20: A split dilator aspiration system as described in any embodiment herein, wherein the handle comprises a first engagement surface, and the dilator has a proximal hub with a second engagement surface configured to engage the first engagement surface to releasably secure the dilator within the catheter.

Embodiment 21: A split dilator aspiration system as described in any embodiment herein, comprising a retention clip carried by the proximal end of the catheter handle.

Embodiment 22: A split dilator aspiration system as described in any embodiment herein, further comprising a retention surface carried by the grip body.

Embodiment 23: A split dilator aspiration system as described in any embodiment herein, wherein the retention surface is on a retention ring configured to engage the retention clip.

Embodiment 24: A split dilator aspiration system as described in any embodiment herein, further comprising a release control, for disengaging the grip body from the catheter handle.

Embodiment 25: A split dilator aspiration system as described in any embodiment herein, wherein the release control comprises at least one push button.

Embodiment 26: A split dilator aspiration system as described in any embodiment herein, further comprising a clot container on the handle.

Embodiment 27: A split dilator aspiration system as described in any embodiment herein, further comprising a hemostasis valve on the handle.

Embodiment 28: A split dilator aspiration system as described in any embodiment herein, wherein the split comprises a weakening in the wall to permit the progressive formation of a slit through the wall to allow lateral escape of the guidewire.

Embodiment 29: A split dilator aspiration system as described in any embodiment herein, wherein the split comprises a pre formed slit completely through the wall.

Embodiment 30: A split dilator aspiration system as described in any embodiment herein, wherein the split extends to a distal endpoint spaced proximally apart from the distal end of the catheter.

Embodiment 31: A split dilator aspiration system as described in any embodiment herein, wherein the distal endpoint is spaced proximally apart within the range of from about 5 cm to about 40 cm from the distal end of the catheter.

Embodiment 32: A split dilator aspiration system as described in any embodiment herein, further comprising a proximal handle on the dilator.

Embodiment 33: A split dilator aspiration system as described in any embodiment herein, wherein the handle comprises a grip body having a first gripping surface and a guidewire exit port configured to direct a guidewire along the first gripping surface.

Embodiment 34: A split dilator aspiration system as described in any embodiment herein, wherein the body comprises a paddle shape with the first gripping surface on a first side and configured to be held between a thumb and forefinger such that a guidewire can be pinned between the thumb and the first gripping surface.

Embodiment 35: A split dilator aspiration system as described in any embodiment herein, further comprising friction enhancing surface structures on the first gripping surface.

Embodiment 36: A split dilator aspiration system as described in any embodiment herein, wherein the friction enhancing surface structures comprise a plurality of ridges.

Embodiment 37: A hemostasis valve, comprising one or more of the following:
a support;
at least a first lever, pivotably carried with respect to the support;
a collapsible tubular sidewall defining a valve lumen carried by the support;
a filament formed into a loop around the tubular sidewall, the filament having at least a first tail portion extending away from the loop to the first lever; and
a first spring configured to move the first lever in a direction that pulls the first tail portion away from the tubular sidewall, reducing the diameter of the valve lumen in response to reducing the diameter of the loop.

Embodiment 38: A hemostasis valve as described in any embodiment herein, further comprising a second lever pivotably carried with respect to the support.

Embodiment 39: A hemostasis valve as described in any embodiment herein, further comprising a second tail portion extending away from the loop and to the second lever.

Embodiment 40: A hemostasis valve as described in any embodiment herein, wherein the first tail portion, second tail portion and loop are one continuous filament.

Embodiment 41: A hemostasis valve as described in any embodiment herein, further comprising a lubricious coating on the filament.

Embodiment 42: A hemostasis valve as described in any embodiment herein, wherein the lubricious coating comprises silicone oil.

Embodiment 43: A hemostasis valve as described in any embodiment herein, wherein the first and second levers are biased in a direction that places the first and second tail portions under sufficient tension to reduce the diameter of the valve lumen and provide a seal around a device extending through the valve.

Embodiment 44: A hemostasis valve as described in any embodiment herein, wherein the first and second levers are biased in a direction that places the first and second tail portions under sufficient tension to close the valve.

Embodiment 45: A hemostasis valve as described in any embodiment herein, wherein the first tail portion is attached to the first lever.

Embodiment 46: A hemostasis valve as described in any embodiment herein, wherein the first tail portion slidably extends around a first fulcrum on the first lever, and is attached to the frame.

Embodiment 47: A hemostasis valve as described in any embodiment herein, wherein the second tail portion slidably extends around a second fulcrum on the second lever, and is attached to the frame.

Embodiment 48: A hemostasis valve as described in any embodiment herein, wherein the first and second fulcrums comprise pins.

Embodiment 49: A hemostasis valve as described in any embodiment herein, mounted on the proximal end of a catheter.

Embodiment 50: A hemostasis valve as described in any embodiment herein, further comprising a connector in communication with the valve lumen, configured for connection to a source of vacuum.

Embodiment 51: A vacuum aspiration system, comprising:
a housing;
a fluid flow path extending through the housing;
a first catheter in fluid communication with the flow path and a connector configured to place a source of aspiration in communication with the flow path;
a clot container carried by the housing; and
a hemostasis valve in the housing, configured to receive a second catheter and direct the second catheter through the first catheter.

Embodiment 52: A vacuum aspiration system as described in any embodiment herein, further comprising a flow regulator, configured to regulate fluid flow through the flow path.

Embodiment 53: A vacuum aspiration system as described in any embodiment herein, wherein at least a portion of the clot container is removably carried by the housing.

Embodiment 54: A vacuum aspiration system as described in any embodiment herein, wherein the clot container comprises a filter membrane spaced apart from a transparent wall.

Embodiment 55: A vacuum aspiration system as described in any embodiment herein, comprising a tubular filter membrane, spaced radially inwardly apart from a transparent outer tubular wall.

Embodiment 56: A vacuum aspiration system as described in any embodiment herein, further comprising an operator actuated control, configured to toggle the flow regulator between a default low flow mode, and a momentary, operator initiated high flow override mode.

Embodiment 57: A vacuum aspiration system as described in any embodiment herein, wherein the operator actuated control comprises a momentary control that places the system into the high flow override mode only when actuated by the operator.

Embodiment 58: A vacuum aspiration system as described in any embodiment herein, further comprising an on—off control which toggles between an off mode and the low flow mode.

Embodiment 59: A vacuum aspiration system as described in any embodiment herein, further comprising a side wall containing the flow path, and an optically transparent window in the side wall.

Embodiment 60: A vacuum aspiration system as described in any embodiment herein, wherein the flow regulator comprises a variable constriction in the flow path.

Embodiment 61: A vacuum aspiration system as described in any embodiment herein, wherein the flow regulator comprises a flexible flow path side wall and an actuator configured to compress the flexible side wall.

Embodiment 62: A vacuum aspiration system as described in any embodiment herein, comprising a flexible filament surrounding the side wall and at least one lever configured to place the filament under tension and close the valve by reducing the diameter of the side wall.

Embodiment 63: A vacuum aspiration system as described in any embodiment herein, further comprising at least one spring, biasing the lever in a direction that closes the valve.

Embodiment 64: A vacuum aspiration system as described in any embodiment herein, wherein the flow regulator comprises a tubing having an inside diameter and length to provide a desired flow rate.

Embodiment 65: A vacuum aspiration system as described in any embodiment herein, wherein the low flow mode aspirates fluid at a rate of no more than about 10cc/second and the high flow mode aspirates fluid at a rate of at least about 15 cc/second in an unobstructed aspiration.

Embodiment 66: A vacuum aspiration system, comprising:
a housing;
a fluid flow path extending through the housing;
a first catheter in fluid communication with the flow path and a connector configured to place a source of aspiration in communication with the flow path;
a flow regulator, configured to regulate fluid flow through the flow path;
a first operator actuated control, configured to toggle the flow regulator between a default, low flow mode, and a momentary, operator initiated high flow override mode; and
a second operator actuated control, configured to turn the fluid flow off.

Embodiment 67: A vacuum aspiration system as described in any embodiment herein, further comprising a port on the housing, in communication with the first connector and configured to guide a second catheter through the housing and into and through the first catheter.

Embodiment 68: A vacuum aspiration system as described in any embodiment herein, further comprising a hemostasis valve carried by the housing, in communication with the port.

Embodiment 69: A vacuum aspiration system as described in any embodiment herein, further comprising a reservoir carried by the housing, for receiving thrombus and blood retrieved through the first catheter.

Embodiment 70: A vacuum aspiration system as described in any embodiment herein, wherein the reservoir comprises a transparent tubular wall releasably caried by the housing.

### Deep Pulsatile Suction

Some embodiments of the vacuum aspiration system disclosed herein can be configured to provide pulsatile suction pressure to the catheter body to more efficiently aspirate a clot material from a patient. Such pulsatile suction pressure is also referred to herein as deep pulse. In some embodiments, the deep pulse can include a rapid increase in flow rate of the fluid through the catheter body to a sustained high flow rate level for a short period of time, followed by a rapid decrease in the flow rate to a negligible or low flow rate level. Thus, the clot can be aspirated during the period of sustained high flow rate and the system can reduce the flow rate through the catheter body to limit blood loss during the procedure. This can all be achieved without changing the pressure that is provided to the catheter by a source of suction and without changing any valve settings, etc. As will be discussed, the aspiration catheter system can be configured such that the portion of the fluid passageway downstream of the clot container of the aspiration catheter system has a significantly smaller diameter or cross-sectional area than the portion of the fluid passageway upstream of the clot container, or so that the portion of the fluid passageway downstream of the clot container of the aspiration catheter system has a flow constrictor (which can be adjustable or can be fixed) that is configured to reduce the diameter or cross-sectional area of such portion, or reduce the flow rate through such portion, as compared to the portion of the fluid passageway upstream of the clot container. In this configuration, in some embodiments, once the clot container is full, the continued aspiration of blood from the patient's body will be significantly reduced due to the flow of the blood through the portion of the fluid passageway downstream of the clot container being more limited by the smaller diameter or cross-sectional area of the fluid passageway downstream of the clot container.

In some embodiments, the vacuum aspiration system, as illustrated in one embodiment in FIG. 26, can have an aspiration catheter 600 assembly that can include a catheter sheath 608 (also referred to herein as an elongate catheter body) extending from a proximal end to a distal end, a housing 606 at the proximal end of the catheter sheath 608, a fluid flow path extending through the housing 606 and the catheter sheath 608 and configured to selectively receive a suction pressure from a source of suction, and an aspiration control valve 642 in fluid communication with the fluid flow path, the aspiration control valve 642 configured to control the suction pressure through the fluid flow path from the source of suction upstream of the aspiration control valve 642.

In some embodiments, the vacuum aspiration system can be configured such that, with the aspiration control valve 642 in a closed state and the source of suction providing the suction pressure to the fluid flow path up to the aspiration valve, when the aspiration control valve 642 is moved to an open state, the source of suction will provide the suction pressure through the fluid flow path upstream of the aspiration control valve 642 such that: (i) a flow rate of a fluid through the fluid flow path upstream of the aspiration control valve 642 increases to a first flow rate range that is greater than zero; (ii) while the aspiration control valve 642 continues to be in the open state, the flow rate of the fluid through the fluid flow path will be maintained at the first flow rate range for a first period or duration of time; and (iii) while the aspiration control valve 642 continues to be in the open state, without any substantial changes (e.g., any changes) to the suction pressure provided by the source of suction (e.g., without turning down or turning off the source of suction), after the first period of time, the flow rate of the fluid through the fluid flow path will drop to a second flow rate range that is greater than zero and that is less than the first flow rate range. The second flow rate can be near zero. In some embodiments of the procedure described above, the aspiration control valve 642 is moved from the closed state to the open state rapidly enough to not affect the flow rate of the fluid through the flow path.

In some embodiments, the second flow rate range is less than 50% or approximately 50% of the first flow rate range, or less than 25% or approximately 25% of the first flow rate range, or less than 15% or approximately 15% of the first flow rate range, or less than 10% or approximately 10% of the first flow rate range, or less than 5% or approximately 5% of the first flow rate range, or of any value, approximate value, or range of values in any of the foregoing ranges.

In some embodiments, this flow rate pattern (i.e., the flow rate increasing to a first flow rate range, the flow rate staying at the first flow rate range for a period of time, and then the flow rate dropping to a second flow rate range) will occur automatically within the vacuum aspiration system. This can be, in some embodiments, due to the fact that a flow of the fluid flowing through the aspiration system is reduced within the aspiration catheter due to physical restraints within the fluid flow path of the aspiration catheter. For example and without limitation, this can be due to the fact that fluid flowing out of the clot container of some embodiments of the aspiration catheter flows through an outlet orifice or passageway downstream of the clot container that is significantly smaller in diameter than an inlet orifice or passageway at the inlet of the clot container, or, in some embodiments, in the passageway upstream of the clot container. In other words, in some embodiments, the minimum diameter of the passageway downstream of the clot container can be significantly smaller than the minimum diameter of the passageway upstream of the clot container such that the maximum possible flow rate of the fluid through the passageway downstream of the clot container is significantly smaller than the maximum possible flow rate of the fluid through the passageway upstream of the clot container.

For example and without limitation, in some embodiments, the passageway downstream of and/or out of the clot container can have a minimum size/diameter that is at least 55% or at least approximately 55% smaller than a minimum size/diameter of the passageway upstream of and coming into the clot container. The portion of the fluid flow path upstream of the clot container, as described above, is meant to refer to the portion of the passageway through the catheter sheath and through the housing 606 up until the clot container. In some embodiments, for example and without limitation, the passageway downstream of and/or out the clot container can have a minimum diameter that is at least 40% or at least approximately 40% smaller, is at least 50% or at least approximately 50% smaller, or that is at least 60% or at least approximately 60% smaller than a minimum diameter of the passageway upstream of the clot container. In some embodiments, for example and without limitation, the passageway downstream of and/or out the clot container can have a minimum diameter that is from 40% or approximately 40% to 80% or approximately 80% smaller, or that is from 50% or approximately 50% to 60% or approximately 60% smaller than a minimum diameter of the passageway upstream of the clot container, or of any value, approximate value, or range of values in any of the foregoing ranges. In some embodiments, the passageway upstream of and into the clot container can have a minimum diameter that is 2.5 times or approximately 2.5 times greater than, or is at least 2 times or is at least approximately 2 times greater than, or is from 1.5 times or approximately 1.5 times to 4 times or approximately 4 times a minimum diameter of the passageway downstream of and out of the clot container, or of any value, approximate value, or range of values in any of the foregoing ranges.

In any embodiments disclosed herein, the first flow rate range (i.e., through the fluid flow path at least upstream of the aspiration control valve 642 that the vacuum aspiration system provides after opening the aspiration control valve 642) can be from 200 ml per second or approximately 200 ml per second to 220 ml per second or approximately 220 ml per second, can be over 200 ml per second (e.g., 210 ml per second or approximately 210 ml per second), or over 180 ml per second, at least when the fluid being aspirated is water. In some embodiments, depending on the type of fluid being aspirated (e.g., blood, water for experimental setups, etc.) the first flow rate range can be greater than 100 ml per second, or greater than 120 ml per second. In some embodiments, when the fluid being aspirated is water, the first flow rate range can be greater than 150 ml per second, greater than 170 ml per second, greater than 180 ml per second, greater than 190 ml per second, greater than 200 ml per second, or greater than 210 ml per second, or from 170 ml per second or approximately 170 ml per second to 210 ml per second or approximately 210 ml per second, or of any value, approximate value, or range of values in any of the foregoing ranges.

In any embodiments disclosed herein, the peak flow rate (i.e., through the fluid flow path that the vacuum aspiration system provides after opening the aspiration control valve 642) can be from 200 ml per second or approximately 200 ml per second to 220 ml per second or approximately 220 ml per second, or can be over 200 ml per second (e.g., 210 ml per second or approximately 210 ml per second), or over 180 ml per second, at least when the fluid being aspirated is water. In some embodiments, wherein the fluid being aspirated is blood or a blood analog, the peak flow rate can be greater than 100 ml per second, or greater than 120 ml per second.

In some embodiments, when the fluid being aspirated is water, the first flow rate range can be greater than 150 ml per second, greater than 170 ml per second, greater than 180 ml per second, greater than 190 ml per second, greater than 200 ml per second, or greater than 210 ml per second, or from 170 ml per second or approximately 170 ml per second to 210 ml per second or approximately 210 ml per second, or of any value, approximate value, or range of values in any of the foregoing ranges. In some embodiments, the blood analog can consist of 49.96% glycerin or approximately 49.96% glycerin, 49.96% water or approximately 49.96% water, and 0.075% xantham gum or approximately 0.075% xantham gum.

In some embodiments, when the fluid aspirated is water, the average flow rate of the water through the fluid flow path during the initial pulse (i.e., from the point when the flow rate increases due to the aspiration control valve being opened through the point where the flow rate has reached its lowest relative value after the initial peak flow rate or that, in some embodiments, corresponds to the clot container being full) is 128 ml per second or approximately 128 ml per second, or is from 100 ml per second or approximately 100 ml per second to 150 ml per second or approximately 150 ml per second, or is from 110 ml per second or approximately 110 ml per second to 140 ml per second or approximately 140 ml per second, or is from 120 ml per second or approximately 120 ml per second to 135 ml per second or approximately 135 ml per second, or is of any value, approximate value, or range of values of any of the foregoing ranges.

As mentioned, some embodiments of the aspiration catheter system is configured such that, after opening the aspiration control valve to provide the suction pressure to the fluid flow pathway of the catheter upstream of the aspiration control valve, the flow rate of fluid through the fluid flow path at least upstream of the aspiration control valve (e.g., upstream of the clot container) is sustained at the first flow rate range for a first period of time. In some embodiments, the first period of time (i.e., the period of time that the flow rate of the fluid through the fluid flow path is maintained at the first flow rate range while the aspiration control valve continues to be in the open state) can be, for example and without limitation, from 0.1 seconds or approximately 0.1 seconds to 0.3 seconds or approximately 0.3 seconds (i.e., greater than or equal to 0.1 seconds or approximately 0.1 seconds and less than or equal to 0.3 seconds or approximately 0.3 seconds, or from 0.1 seconds or approximately 0.1 seconds to 0.25 seconds or approximately 0.25 seconds (i.e., greater than or equal to 0.1 seconds or approximately 0.1 seconds and less than 0.25 seconds or approximately 0.25 seconds), or from 0.1 seconds or approximately 0.1 seconds to 0.2 seconds or approximately 0.2 seconds (i.e., greater than or equal to 0.1 seconds or approximately 0.1 seconds and less than 0.2 seconds or approximately 0.2 seconds).

In this configuration of some embodiments, the vacuum aspiration system can be configured to provide a rapid drop-off of the suction provided through the catheter such that the flow rate of the fluid through the fluid flow path drops below 20 mL per second within 0.4 or approximately 0.4 seconds, or within 0.3 seconds or approximately 0.3 seconds, or within 0.2 seconds or approximately 0.2 seconds after the flow rate of the fluid through the fluid flow path first reaches the peak flow rate, while the aspiration control valve 642 remains in an open state and the suction pressure continues to be applied.

In some embodiments, the total volume of the fluid aspirated through the catheter sheath 608 when the flow rate of the fluid through the fluid flow path drops to the second flow rate range is 20 ml or approximately 20 ml, or is 16 ml or approximately 16 ml, or 17.5 ml or approximately 17.5 ml, or 18 ml or approximately 18 ml, or 19 ml or approximately 19 ml, or from 10 ml or approximately 10 ml to 30 ml or approximately 30 ml, or from 15 ml or approximately 15 ml to 25 ml or approximately 25 ml, or of any value, approximate value, or range of values in any of the foregoing ranges. The foregoing values and ranges can be for any aspiration catheter size, including without limitation a 24 Fr catheter or a 16 Fr catheter, and where in the catheter can be primed before fluid is aspirated.

In some embodiments disclosed herein, the source of suction can be a vacuum pump or a syringe, or other source of suction. In some embodiments, the vacuum aspiration system can include a suction conduit 618 in communication with the fluid flow path and configured to be fluidically coupled with the source of suction. In any embodiments, the source of suction applies a suction pressure of −28 inHg or approximately −28 inHg, or from −26 inHg or approximately −26 inHg to −29.92 inHg or approximately −29.92 inHg. In some embodiments, the values listed above are for suction pressure applied at sea level and can be adjusted based on elevation.

In some embodiments, the vacuum aspiration system can be configured to provide a rapid burst of suction through the catheter so that the catheter reaches a peak flow rate of a fluid through the fluid flow path within 0.06 seconds, at least when the fluid is fluid is water and the catheter is primed before aspiration begins. In some embodiments, the vacuum aspiration system can be configured to provide a rapid burst of suction through the catheter so that the catheter reaches a peak flow rate of a fluid through the fluid flow path at approximately 0.055 seconds, at least when the fluid is water and the catheter is primed before aspiration begins. In some embodiments, the vacuum aspiration system can be configured to provide a rapid burst of suction through the catheter so that the catheter reaches a peak flow rate of a fluid through the fluid flow path within 0.03 seconds to 0.06 seconds, at least when the fluid is water and the catheter is primed before aspiration begins.

As mentioned, in some embodiments, the aspiration control valve 642 can be configured to move between a first position wherein the aspiration control valve 642 is closed and a second position wherein the aspiration control valve 642 is open, and wherein suction is provided to the fluid flow path by moving the aspiration control valve 642 to the second position, thereby providing the suction from the source of suction to the fluid flow path. The aspiration control valve 642 can have a lever that a user can grasp or move between a first and a second position.

As mentioned, any embodiments of the aspiration catheter disclosed herein can include a clot container coupled with the housing 606, the clot container being in fluid communication with the fluid flow path. In some embodiments, the clot container has a filter therein configured to inhibit (e.g., prevent) a clot material from passing through the filter such that clot material remains within the clot container outside of the filter. The clot container can have an outlet that is in fluid communication with the fluid flow path.

In some embodiments, the fluid flow path passes through a suction conduit 618 that fluidically couples to the source of suction. In some embodiments, the suction conduit 618 has an internal diameter (D8) of 2.67 mm, or approximately 2.67 mm, or from 2 mm or approximately 2 mm to 4 mm or approximately 4 mm, or from 2.5 mm or approximately 2.5 mm to 3 mm or approximately 3 mm, or of any value, approximate value, or range of values in any of the foregoing ranges. In some embodiments, the portion of the suction conduit 618 extending from where the conduit 618 exits the housing to the port can have a length of 16.5 cm or approximately 16.5 cm, or from 12 cm or approximately 12 cm to 22 cm or approximately 22 cm, or from 14 cm or approximately 14 cm to 19 cm or approximately 19 cm, or of any value, approximate value, or range of values in any of the foregoing ranges. In some embodiments, the portion of the suction conduit 618 inside the housing to the end of the conduit 618 near the clot container can have a length of 12 cm or approximately 12 cm, or from 8 cm or approximately 8 cm to 16 cm or approximately 16 cm, or from 10 cm or approximately 10 cm to 14 cm or approximately 14 cm, or of any value, approximate value, or range of values in any of the foregoing ranges.

In some embodiments, the outlet has a conduit that is in fluid communication with the fluid flow path, wherein the suction conduit 618 is downstream of the clot container. In some embodiments, the conduit of the outlet of the clot container has a minimum internal diameter (D6) of 2 mm, or approximately 2 mm, or from 1 mm or approximately 1 mm to 3 mm or approximately 3 mm, or from 1.5 mm or approximately 1.5 mm to 2.5 mm or approximately 2.5 mm, or of any value, approximate value, or range of values in any of the foregoing ranges.

In some embodiments, the conduit of the outlet of the clot container has a first internal diameter (D6) of 2 mm, or approximately 2 mm, or from 1 mm or approximately 1 mm to 3 mm or approximately 3 mm, or from 1.5 mm or approximately 1.5 mm to 2.5 mm or approximately 2.5 mm, or of any value, approximate value, or range of values in any of the foregoing ranges, and a second internal diameter (D7) of 2.25 mm, or approximately 2.25 mm, or from 1.5 mm or approximately 1.5 mm to 3 mm or approximately 3 mm, or from 2 mm or approximately 2 mm to 2.5 mm or approximately 2.5 mm, or of any value, approximate value, or range of values in any of the foregoing ranges.

In some embodiments, the clot container can have an inlet into the clot container having an internal diameter of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges, and a an outlet from the clot container downstream of the inlet into the clot container, the outlet having an internal diameter of 2.8 mm, or approximately 2.8 mm, or from 1.8 mm or approximately 1.8 mm to 3.8 mm or approximately 3.8 mm, or from 2.5 mm or approximately 2.5 mm to 3.1 mm or approximately 3.1 mm, or of any value, approximate value, or range of values in any of the foregoing ranges.

In some embodiments, the fluid flow pathway comprises a conduit extending from the aspiration control valve 642 to the inlet of the clot container having an internal diameter (D5) of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges. The fluid flow pathway can include a conduit extending from the aspiration control valve 642 upstream of the aspiration control valve 642 toward a proximal end of the catheter sheath 608, the conduit extending from the aspiration control valve 642 having an internal diameter (D3, D4) through the conduit of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges.

In some embodiments, the conduit 671 can be configured to join or merge with the catheter body 608 in a way that reduces the likelihood of a clot or other material clogging or becoming lodged in the lumen 672 through the conduit 671, e.g., so as to provide a smooth transition through a fluid passageway that includes the catheter and the conduit 671, particularly at the junction between the conduit 671 and the catheter body 608. For example and without limitation, the conduit 671 can have a first portion 671a that has an end portion that merges with or connects to the catheter and a second portion 671b in communication with the first portion 671a that extends toward a clot container (such as clot container 620). In some embodiments, the first portion 671a of the conduit 671, e.g., the end portion of the conduit 671, can form a smooth transition with the catheter.

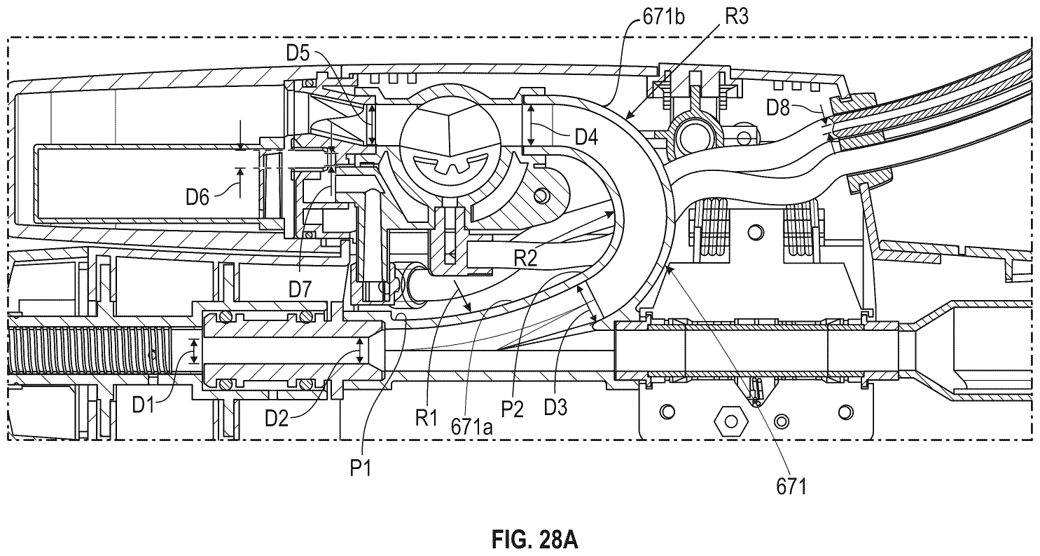

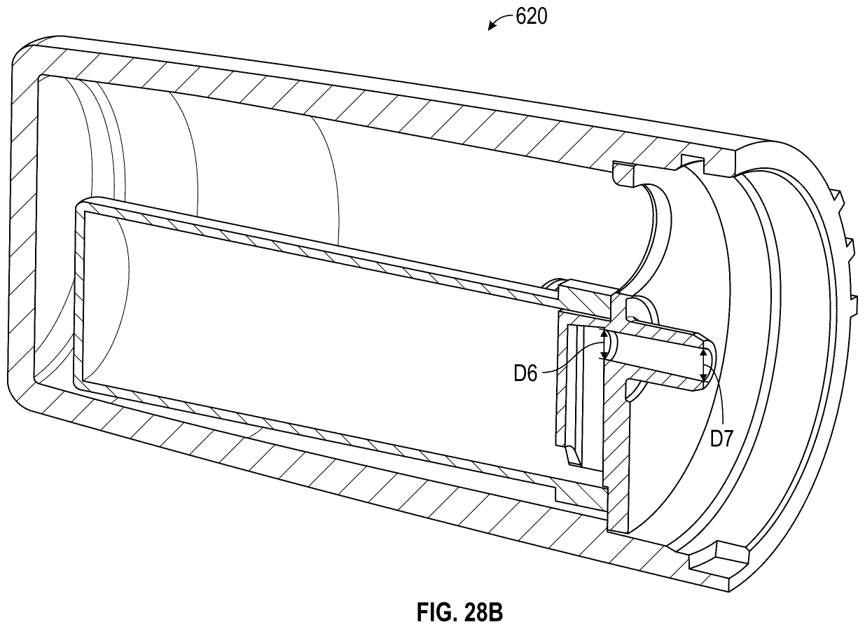

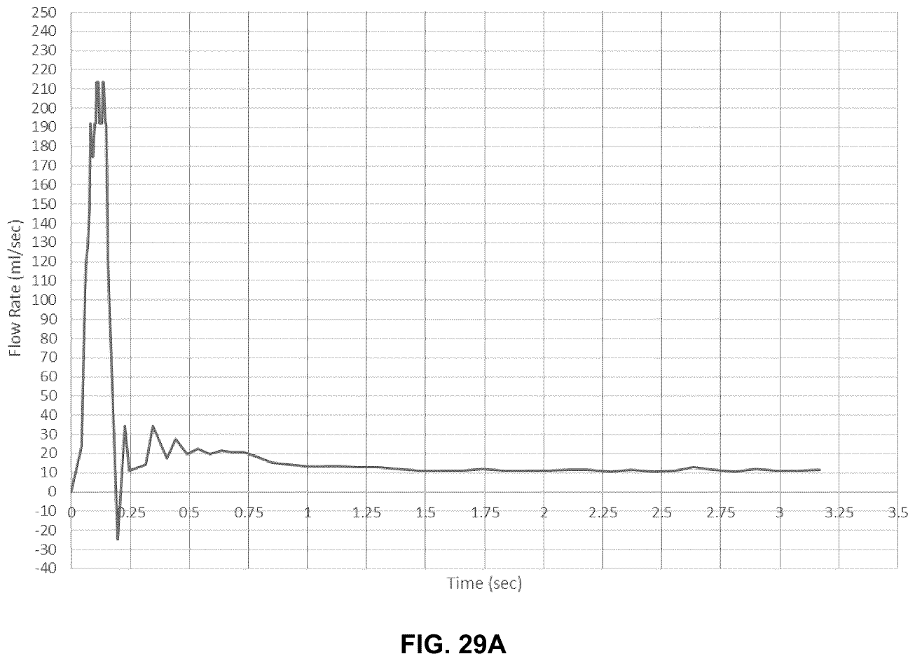

In some embodiments, the end portion of the conduit 671 (e.g., the end portion of the first portion 671a of the conduit 671) can define a takeoff angle (i.e., the angle of departure of the conduit 671 away from the catheter body 608) that can be very small or can be initially zero and can gradually increase to improve the flow characteristics of blood, clot material, and/or other material through the conduit 671. For example and without limitation, some embodiments of the handle 606 can be configured such that the departure or takeoff angle of the first portion 671a of the conduit 671 away from the catheter body 608 such as at point P1, as shown in FIG. 28A, is zero—i.e., the end portion of the conduit 671 can be tangent to the catheter at point P1. In some embodiments, the conduit 671 can asymptotically depart from or angle away from the catheter body 608 so that a wall portion of the conduit 671 is tangent with the catheter body 608 at point P1, so as to provide a smooth transition away from the catheter.

Additionally, the end portion of the conduit 671 can have a gradual curvature, such as in the first portion 671a of the conduit 671. For example and without limitation, the first portion 671a of the conduit 671 can have a radius of curvature (shown as R1 in FIG. 28A) that is 2.8 inches or approximately 2.8 inches, or is from 2 inches or approximately 2 inches to 4 inches or approximately 4 inches, or is from 2.5 inches or approximately 2.5 inches to 3.5 inches or approximately 3.5 inches, or that can be 71 mm or approximately 71 mm, or that is from 60 mm or approximately 60 mm to 80 mm or approximately 80 mm, or that can be any of the values, approximate values, or ranges of values in any of the foregoing ranges.

In some embodiments, the first portion 671a of the conduit 671 can have a radius of curvature (shown as R1 in FIG. 28A) that is 10 times or approximately 10 times greater than the internal diameter (D3, as illustrated in FIG. 28A) through the conduit 671, or from 5 times or approximately 5 times greater to 15 times or approximately 15 times greater than the internal diameter D3 through the conduit 671, or from 7 times or approximately 7 times greater to 13 times or approximately 13 times greater than the internal diameter D3 through the conduit 671, or at least 5 times greater, or at least 10 times greater, or at least 15 times greater, than the internal diameter D3 through the conduit 671. In some embodiments, the first portion 671a of the conduit 671 can extend from point P1 to point P2. In some embodiments, the radius of curvature R1 can be an inside radius of curvature of the first portion 671a of the conduit.

In some embodiments, the second portion 671b of the conduit 671 can extend from the first portion 671a of the conduit 671 toward the clot container (such as clot container 620). The second portion 671b of the conduit 671 can have a radius of curvature that is different than the radius of curvature R1 of the first portion 671a of the conduit 671. For example and without limitation, the second portion 671b can have an inside radius of curvature, shown as R2 in FIG. 28A and an outside radius of curvature, shown as R3 in FIG. 28A. In some embodiments, the inside curvature of radius R2 can be smaller than, or significantly smaller than, the radius of curvature R1 of the first portion 671a of the conduit 671. For example and without limitation, the radius of curvature R2 of the second portion 671b of the conduit 671 can be 0.4 inches, or approximately 0.4 inches, or is from 0.2 inches or approximately 0.2 inches to 0.6 inches or approximately 0.6 inches, or is from 0.3 inches or approximately 0.3 inches to 0.5 inches or approximately 0.5 inches, or that can be 9.9 mm or approximately 9.9 mm, or 10 mm or approximately 10 mm, or from 8 mm or approximately 8 mm to 12 mm or approximately 12 mm, or from 6 mm or approximately 6 mm to 14 mm or approximately 14 mm, or of any of the values, approximate values, or ranges of values in any of the foregoing ranges.

In some embodiments, the first portion 671a of the conduit 671 can have a radius of curvature (shown as R1 in FIG. 28A) that is 7 times or approximately 7 times greater than the radius of curvature R2 of the second portion 671b of the conduit 671, or is from 6 times or approximately 6 times greater to 8 times or approximately 8 times greater than the radius of curvature R2 of the second portion 671b of the conduit 671, or is from 5 times or approximately 5 times greater to 10 times or approximately 10 times greater than the radius of curvature R2 of the second portion 671b of the conduit 671, or of any values, approximate values, or ranges of values in the foregoing ranges.

In some embodiments, the catheter can have a sealing element at a proximal end of the catheter sheath 608, the sealing element having a passageway therethrough that is part of the fluid flow path and that has a minimum internal diameter (D1) of 4.67 mm, or approximately 4.67 mm, or from 3.7 mm or approximately 3.7 mm to 5.7 mm or approximately 5.7 mm, or from 4.2 mm or approximately 4.2 mm to 5.2 mm or approximately 5.2 mm, or of any value, approximate value, or range of values in any of the foregoing ranges. In some embodiments, the sealing element has a first end that is distal to a second end, the first end being upstream of the first end in the fluid pathway. In some embodiments, the minimum internal diameter (D1) is at the first end and the second end has an internal diameter (D2) of 5.14 mm, or approximately 5.14 mm, or from 4 mm or approximately 4 mm to 6.5 mm or approximately 6.5 mm, or from 4.5 mm or approximately 4.5 mm to 6 mm or approximately 6 mm, or of any value, approximate value, or range of values in any of the foregoing ranges.

In some embodiments, the flow rate drops from the first flow rate to the second flow rate when the clot container becomes full with the fluid that has been aspirated through the catheter sheath 608. In some embodiments, this can occur within 0.3 seconds of when the first peak flow rate is reached, or from 0.1 seconds to 0.25 seconds (e.g., without limitation, no more than from 0.1 seconds to 0.25 seconds), or from 0.1 seconds to 0.2 seconds (e.g., without limitation, no more than from 0.1 seconds to 0.2 seconds) of when the first peak flow rate is achieved, or at any value, approximate value, or range of values within any of the foregoing ranges. In some embodiments, the fluid can be water. The fluid can be blood also, though the time to fill the clot container would be, in some embodiments, longer than for water.

In some embodiments, the clot container has a volume of 17.7 ml or approximately 17.7 ml, or from 15 ml or approximately 15 ml to 20 ml or approximately 20 ml, or from 17 ml or approximately 17 ml to 19 ml or approximately 19 ml, or of any value, approximate value, or range of values in any of the foregoing ranges. In some embodiments, the clot container has a volume of 40 ml or approximately 40 ml, or a volume of 60 ml or approximately 60 ml, or from 30 ml or approximately 30 ml to 80 ml or approximately 80 ml, or from 40 ml or approximately 40 ml to 70 ml or approximately 70 ml, or of any value, approximate value, or range of values in any of the foregoing ranges.

In some embodiments, the flow rate drops from the first flow rate to the second flow rate in 0.05 seconds, approximately 0.05 seconds, or less than 0.05 seconds, or in 0.075 seconds, approximately 0.075 seconds, or less than 0.075 seconds, or from 0.025 seconds or approximately 0.025 seconds to 0.075 seconds or approximately 0.075 seconds, or from 0.04 seconds or approximately 0.04 seconds to 0.06 seconds or approximately 0.06 seconds, or of any value, approximate value, or range of values in any of the foregoing ranges. In some embodiments, the first period of time can be increased by increasing the volume of the clot container and/or a volume of the fluid flow path upstream of the clot container. In some embodiments, dropping the flow rate of fluid through the fluid flow path to the second flow rate after the first period of time reduces (e.g., substantially reduces) a loss of blood from a patient during a thrombectomy procedure.

In some embodiments, a volume of the fluid flow path downstream of the catheter sheath 608 and downstream to and including the clot container is 22.6 ml or approximately 22.6 ml, or from 18 ml or approximately 18 ml to 25 ml or approximately 25 ml, or from 20 ml or approximately 20 ml to 25 ml or approximately 25 ml, or of any value, approximate value, or range of values in any of the foregoing ranges. In some embodiments, a volume of the fluid pathway from and including the suction conduit 618 upstream up to the clot container is 1.8 ml or approximately 1.8 ml, or from 1.5 ml or approximately 1.5 ml to 2.0 ml or approximately 2.0 ml, or from 1.7 ml or approximately 1.7 ml to 1.9 ml or approximately 1.9 ml, or of any value, approximate value, or range of values in any of the foregoing ranges.

In some embodiments, another conduit (referred to herein as the second conduit) can be coupled with the suction conduit 618 and can be used to fluidically couple the suction conduit 618 to a collection canister and/or source of suction. In some embodiments, this conduit can have a similar inner diameter as compared to the suction conduit 618 and can have a length of 106 inches, approximately 106 inches, from 50 inches or approximately 50 inches to 120 inches or approximately 120 inches, from 80 inches or approximately 80 inches to 110 inches or approximately 110 inches, or of any value, approximate value, or range of values in any of the foregoing ranges. In some embodiments, this second conduit can limit the flow rate of the fluid through downstream of the clot container and can contribute to the flow rate dynamics (e.g., without limitation, the deep pulse characteristics) described herein.

In some embodiments, the vacuum aspiration system can be configured such that, with the aspiration control valve 642 in a closed state and the source of suction providing the suction pressure to the fluid flow path up to the aspiration valve, when the aspiration control valve 642 is moved to an open state, the source of suction will provide the suction pressure through the fluid flow path upstream of the aspiration control valve 642 such that: (i) a flow rate of a fluid through the fluid flow path increases to a first flow rate range that is greater than zero; (ii) while the aspiration control valve 642 continues to be in the open state, the flow rate of the fluid through the fluid flow path is maintained at the first flow rate range until a first volume of the fluid has been aspirated through the catheter sheath 608; and (iii) while the aspiration control valve 642 continues to be in the open state, after the first volume of the fluid has been aspirated through the catheter sheath 608, the flow rate of the fluid through the fluid flow path drops to a second flow rate range that is greater than zero but less than the first flow rate range.

In some embodiments, the vacuum aspiration system can be configured such that, with the aspiration control valve 642 in a closed state, thereby inhibiting (e.g., preventing) a flow of a fluid through the aspiration control valve 642, and the source of suction providing suction to the fluid flow path to the aspiration control valve 642, when the aspiration control valve 642 is moved to an open state, the source of suction will provide a rapid burst of suction through the catheter such that a peak flow rate (i.e., maximum flow rate) of a fluid through the fluid flow path is achieved within 0.08 seconds or approximately 0.08 seconds after suction is provided to the fluid flow path, or is achieved within 0.1 seconds or approximately 0.1 seconds, or from 0.05 seconds or approximately 0.05 seconds to 0.12 seconds or approximately 0.12 seconds after suction is provided to the fluid flow path.

Also disclosed herein are embodiments of a method of aspirating a fluid through a catheter of an aspiration system. In some embodiments, the method includes: (i) positioning an aspiration control valve 642 of the catheter in a closed position; (ii) with the aspiration control valve 642 in the closed position and a fluid flow path of the catheter in fluid communication with the fluid, applying a suction pressure to a fluid flow path of the catheter; and (iii) moving the aspiration control valve 642 to an open position to aspirate the fluid through the fluid flow path of the catheter. In some embodiments, a flow rate of the fluid through the fluid flow path increases to a first flow rate range that is greater than 50 ml per second when the aspiration control valve is moved the open position. In some embodiments, the method further includes, when the flow rate of the fluid through the fluid flow path decreases below a second value, moving the aspiration control valve 642 back to the closed position. In some embodiments, as has been described herein, the catheter can be configured to decrease the flow rate of the fluid through the fluid flow path without changing the suction pressure being applied to the fluid flow path of the catheter. In some embodiments, the catheter can be configured to decrease the flow rate of the fluid through the fluid flow path to the second value of the flow rate range without making any changes to the aspiration system.

In some embodiments, the first flow rate range is greater than 100 ml per second or approximately 100 ml per second, or is greater than 150 ml per second or approximately 150 ml per second, or is greater than 180 ml per second or approximately 180 ml per second, or is greater than 190 ml per second or approximately 190 ml per second, or is greater than 200 ml per second or approximately 200 ml per second, or is from 100 ml per second or is approximately 100 ml per second to 200 ml per second or approximately 200 ml per second, or is from 150 ml per second or approximately 150 ml per second to 200 ml per second or approximately 200 ml per second, or of any value, approximate value, or range of values in any of the foregoing ranges. In any embodiments disclosed herein, the fluid can be water. In any embodiments disclosed herein, the fluid can be blood.

In some embodiments, the second value (i.e., the second flow rate value that the aspiration flow rate decreases to) can be between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges.

Also disclosed herein are embodiments of a method of aspirating a clot material during a thrombectomy procedure, that can include positioning an aspiration control valve 642 of the aspiration catheter 600 in a closed position, applying a suction pressure to a fluid flow path of the catheter, positioning an aspiration catheter 600 within a predetermined distance of a clot within a patient's vasculature, moving the aspiration control valve 642 to an open position to aspirate the clot through the fluid flow path of the aspiration catheter 600 wherein a flow rate of a fluid through the fluid flow path upstream of the aspiration control valve 642 increases to a first flow rate range that is greater than zero, when the flow rate of the fluid through the fluid flow path decreases below a second flow rate value or within a first period of time (as discussed above), moving the aspiration control valve 642 back to the closed position, withdrawing the aspiration catheter 600 a predetermined distance, and, after withdrawing the aspiration catheter 600 the predetermined distance, moving the aspiration control valve 642 again to the open position to continue to aspirate the clot through the fluid flow path of the aspiration catheter 600, wherein the flow rate of the fluid through the fluid flow path upstream of the aspiration control valve 642 increases again to the first flow rate range.

In some embodiments, the method can further include, when the flow rate of the fluid through the fluid flow path again decreases below the second flow rate value, moving the aspiration control valve 642 back to the closed position. In some embodiments, the method can further include withdrawing the aspiration catheter 600 a second predetermined distance and, after withdrawing the aspiration catheter 600 the second predetermined distance, moving the aspiration control valve 642 again to the open position to continue to aspirate the clot through the fluid flow path of the aspiration catheter 600, wherein the flow rate of the fluid through the fluid flow path upstream of the aspiration control valve 642 increases again to the first flow rate range or near the first flow rate range. In some embodiments, the method can further include, when the flow rate of the fluid through the fluid flow path decreases below the second flow rate value for a third time, moving the aspiration control valve 642 back to the closed position.

In some embodiments, the method can further include withdrawing the aspiration catheter 600 by a third predetermined distance and, after withdrawing the aspiration catheter 600 the third predetermined distance, moving the aspiration control valve 642 again to the open position to continue to aspirate the clot through the fluid flow path of the aspiration catheter 600, wherein the flow rate of the fluid through the fluid flow path upstream of the aspiration control valve 642 increases again to the first flow rate range or near the first flow rate range. In some embodiments, the method can further include, when the flow rate of the fluid through the fluid flow path decreases below the second flow rate value for a third time, moving the aspiration control valve 642 back to the closed position. In some embodiments, this procedure can be repeated again as many times as desired or needed for a more complete aspiration of the clot material.

In other embodiments, the user can distally advance the aspiration catheter 600 a predetermined distance, or a second predetermined distance, or a third predetermined distance, or otherwise (instead of withdrawing the aspiration catheter 600 a predetermined distance, a second predetermined distance, or a third predetermined distance, or otherwise, respectively) and, after advancing the aspiration catheter 600 the predetermined distance, the second predetermined distance, and/or the third predetermined distance, or otherwise, move the aspiration control valve 642 again to the open position to continue to aspirate the clot through the fluid flow path of the aspiration catheter 600, wherein the flow rate of the fluid through the fluid flow path upstream of the aspiration control valve 642 increases again to the first flow rate range.

In any embodiments disclosed herein, the predetermined distance (which can include the second predetermined distance and/or the third predetermined distance) can be 0 cm or approximately 0 cm, 2 cm or approximately 2 cm, 3 cm or approximately 3 cm or no greater than 3 cm, 4 cm or approximately 4 cm or no greater than 4 cm, 5 cm, approximately 5 cm, or greater than 5 cm, or from 0 cm or approximately 0 cm to 5 cm, approximately 5 cm, or more than 5 cm, or from 1 cm or approximately 1 cm to 4 cm or approximately 4 cm, or from 1 cm or approximately 1 cm to 3 cm or approximately 3 cm, or of any of the values, approximate values or range of values of any of the foregoing ranges. In any embodiments disclosed herein, the predetermined distance, the second predetermined distance, and the third predetermined distance can each be the same or can each be different. In any embodiments of the methods disclosed herein, the clot container or filter can be cleared or cleaned to remove any clot material that may be on the filter or otherwise be in the clot container at any step or point during the procedure.

In some embodiments, the first flow rate range can be greater than 50 ml per second or approximately 50 ml per second, or can be greater than 60 ml per second or approximately 60 ml per second, or can be from 40 ml per second to 120 ml per second, or can be from 50 ml per second to 100 ml per second, or any values, approximate values, or ranges of values within any of the foregoing ranges, when the fluid is blood. In some embodiments, the second flow rate value is from 2 ml per second, approximately 2 ml per second, or less than 2 ml per second and 20 ml per second or approximately 20 ml per second, or from 5 ml per second or approximately 5 ml per second and 10 ml per second or approximately 10 ml per second, or of any value, approximate value, or range of values in any of the foregoing ranges when the fluid is blood.

In some embodiments, the flow rate of the fluid through the fluid flow path decreases below the second flow rate value when a clot container of the aspiration catheter 600 becomes full with the fluid or the fluid and the clot material. In some embodiments, the aspiration catheter 600 can be configured to decrease the flow rate of the fluid through the fluid flow path below the second flow rate value without any user input.

### Experiments:

The following describes a series of experiments that were performed for aspiration catheter embodiments disclosed herein. A 100 ml graduated cylinder was filled with 100 ml of water or a blood analog liquid, as identified below. The blood analog liquid (referred to herein as a blood analog) and water and are collectively referred to as a liquid. The experimental setup also included a camera pointed at the graduated cylinder to measure the volume of the water within the graduated cylinder over time. For each of the following experiments, the catheter was fully primed with the liquid such that the fluid flow path was filled completely with the liquid entirely through the catheter sheath and from the catheter sheath all the way to the aspiration control valve. Pressure applied by the suction source for all experiments was measured at −28 inHg. The liquid being aspirated and used to prime the catheter was water or the blood analog, as identified below, at room temperature and was held within the graduated cylinder with an outlet tube passing through a bottom surface of the graduated cylinder and connected to a distal end of the catheter sheath for all experiments. A camera configured to capture images at 960 frames per second was used to measure the volume of the liquid within the graduated cylinder throughout the course of all of the experiments.

The procedure for the following four experiments (i.e., Experiment 1 through Experiment 4) is as follows. The listing of the steps of the procedure below does not indicate a particular order of the steps, which may be able to be done in other orders, unless specified below:
i. Filling the graduated cylinder with water at room temperature or near room temperature and recording the initial volume of the water in the graduated cylinder;
ii. Priming the catheter with water;
iii. Activating the source of suction, wherein the source of suction is in fluid communication with the fluid flow path up to the aspiration control valve;
iv. Moving the aspiration control valve to the open position to aspirate the liquid from the graduated cylinder;
v. Recording the volume of the water in the graduated cylinder at predetermined time intervals; and
vi. Calculating the volume of the water that has been aspirated from the graduated cylinder at the predetermined time intervals by subtracting the volume of water in the graduated cylinder at each predetermined time from the initial volume of water in the graduated cylinder.

After the desired amount of liquid has been aspirated through the catheter, the aspiration control valve can then be closed and the suction source can be turned off, in any desired order. Note that, in some embodiments, moving the aspiration control valve to the open position entails moving the lever of the aspiration control valve to the open position quickly.

### Experiment 1

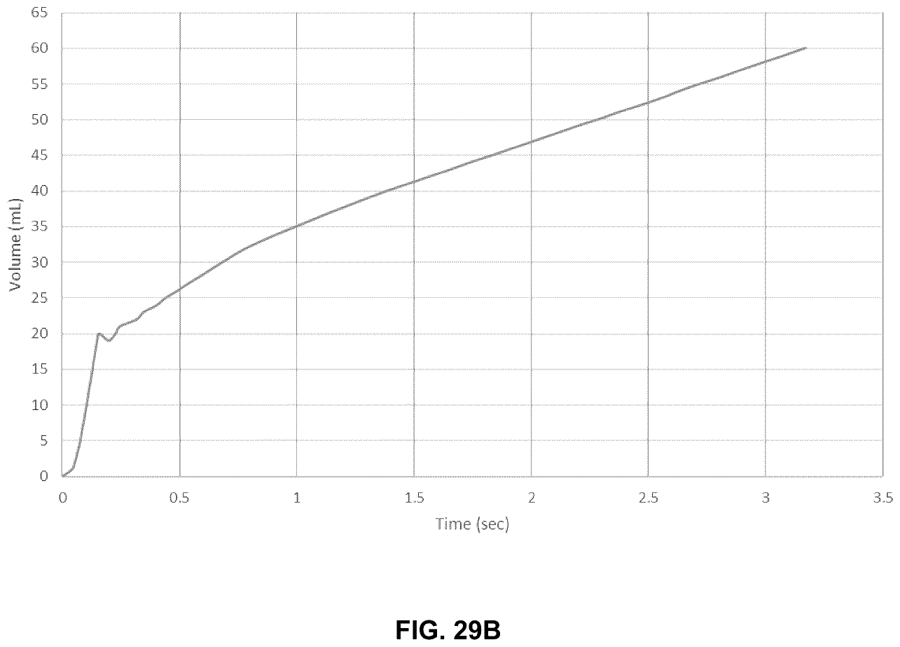

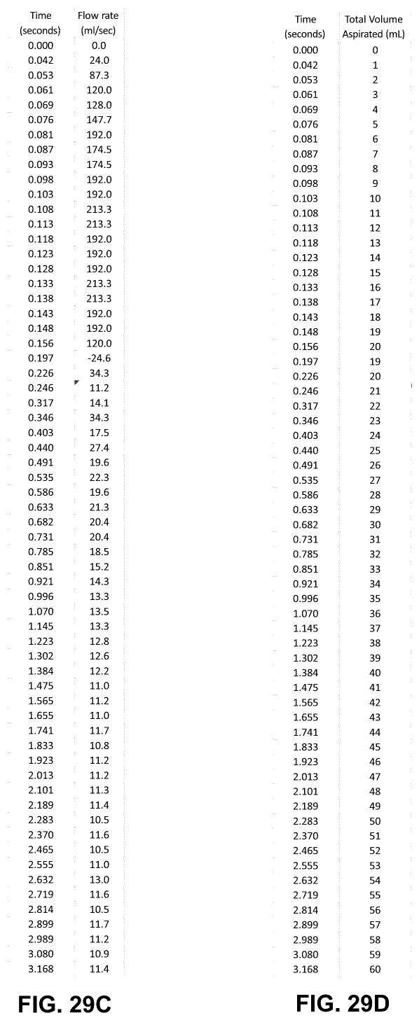

FIGS. 29A and 29B show the results of the first experiment performed on an embodiment of an aspiration catheter as disclosed herein and using an Imperative Care pump as the suction source, according to the setup details and procedure described above. The aspiration catheter is a 24 Fr catheter. The Imperative Care pump provides suction to the catheter through a tube having a length that is approximately 106 inches (for example, without limitation, 106 inches) and that has the same inner diameter as aspiration tubing 618.

FIG. 29A shows the flow rate in ml per second over time. With reference to FIG. 29A, the catheter has a flow rate of 0 ml per second at the beginning of the experiment when the aspiration control valve is in the off or closed position. This corresponds to time zero on the horizontal scale. The flow rate of the water through the catheter then quickly increases to the first flow rate range. In the embodiment of the 24 Fr aspiration catheter tested in Experiment 1, the maximum flow rate measured was 213 ml per second, the first flow rate range measured ranged from 192 ml per second to 213 ml per second over a 0.05 second interval, starting at 0.098 seconds and lasting through 0.148 seconds. The flow rate started to decrease after 0.148 seconds. The measured flow rate eventually decreased to a second flow rate range that ranged from 10.5 ml per second to 11.7 ml per second after 1.45 seconds. However, it is believed that the actual flow rate was within or close to this second flow rate range between 0.156 seconds and 0.197 seconds but that the perturbations in the graduated cylinder caused the flow rate measurements to dip below zero at 0.197 seconds and oscillate for approximately 0.5 seconds before staying within the second flow rate range described above.

FIG. 29B shows the total volume aspirated in ml over time (in seconds).

FIGS. 29C and 29D show the data used to generate the plots of FIGS. 29A and 29B, respectively.

### Experiment 2

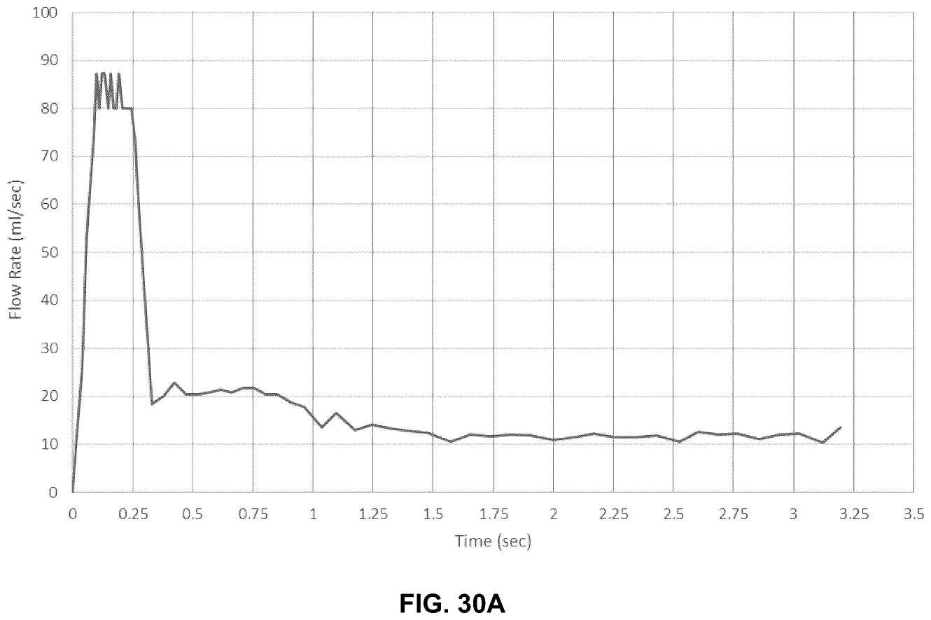

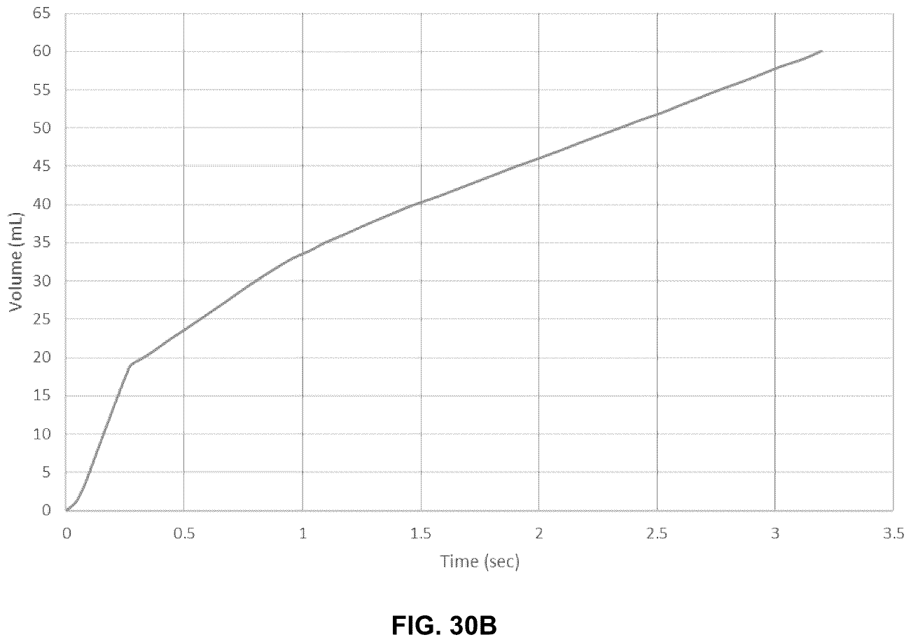

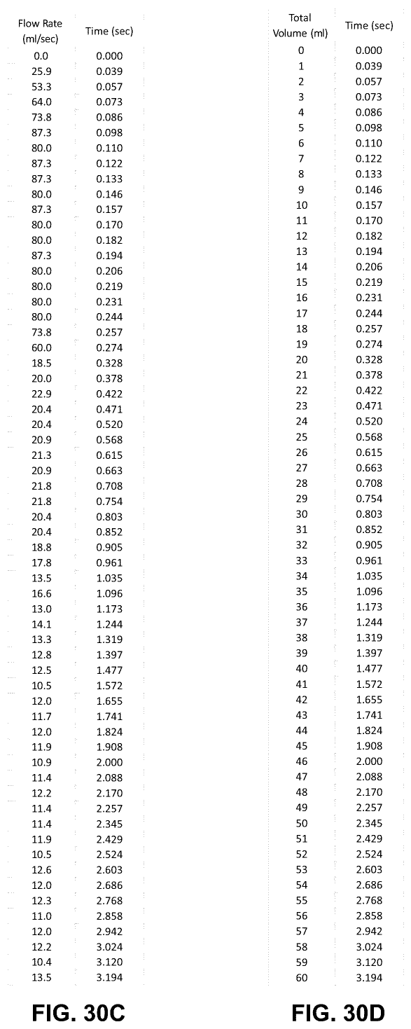

FIGS. 30A and 30B show the results of another experiment performed on an embodiment of an aspiration catheter as disclosed herein and using an Imperative Care pump as the suction source, according to the setup details and procedure described above. The aspiration catheter is a 16 Fr catheter. The Imperative Care pump provides suction to the catheter through a tube having a length that is approximately 106 inches (for example, without limitation, 106 inches) and that has the same inner diameter as aspiration tubing 618.

FIG. 30A shows the flow rate in ml per second over time. With reference to FIG. 30A, the catheter has a flow rate of 0 ml per second at the beginning of the experiment when the aspiration control valve is in the off or closed position. This corresponds to time zero on the horizontal scale. The flow rate of the water through the catheter then quickly increases to the first flow rate range. In the embodiment of the 16 Fr aspiration catheter tested in Experiment 2, the maximum flow rate measured was 87.3 ml per second, the first flow rate range measured ranged from 80.0 ml per second to 87.3 ml per second over a 0.05 second interval, starting at 0.098 seconds and lasting through 0.146 seconds. The flow rate started to decrease after 0.146 seconds and dropped to 18.5 ml per second at 0.328 seconds after aspiration pressure was applied to the fluid flow path. The measured flow rate decreased to a flow rate range of from 10.5 ml per second to 13.5 ml per second after 1.32 seconds. Perturbations in the graduated cylinder may have caused some fluctuation in the flow rate measurements.

FIG. 30B shows the total volume aspirated in ml over time (in seconds).

FIGS. 30C and 30D show the data used to generate the plots of FIGS. 30A and 30B, respectively.

### Experiment 3

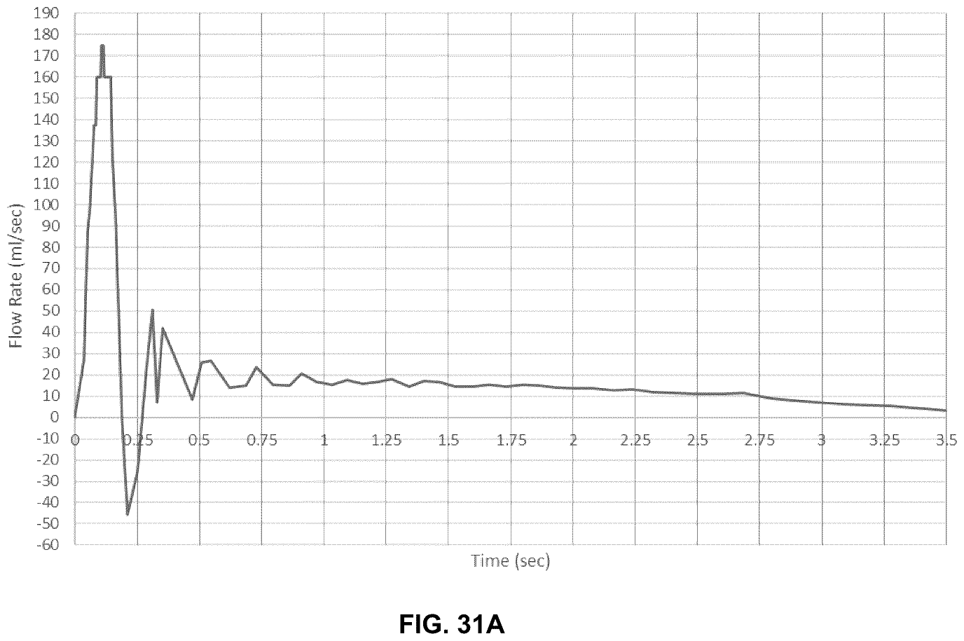

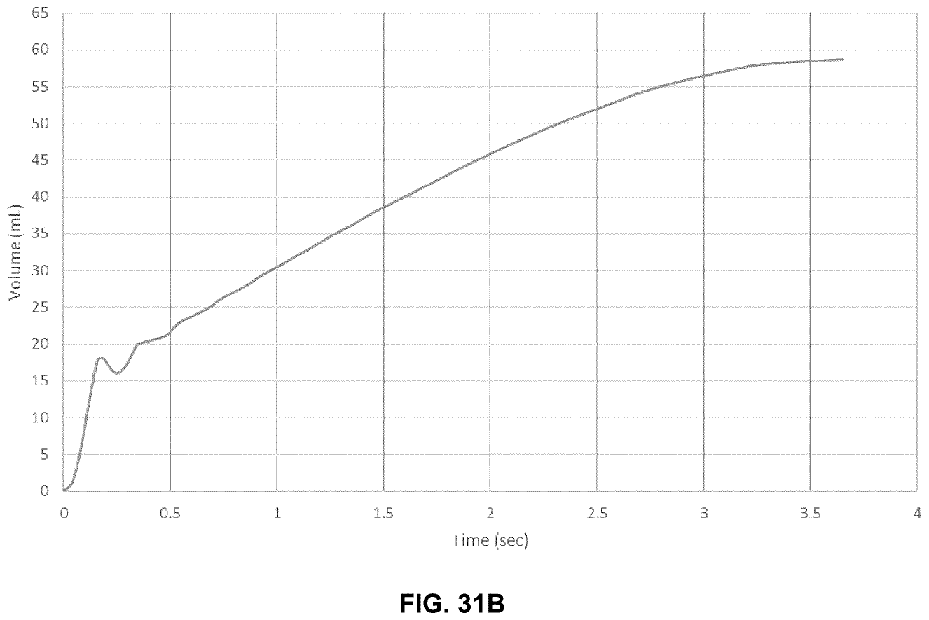

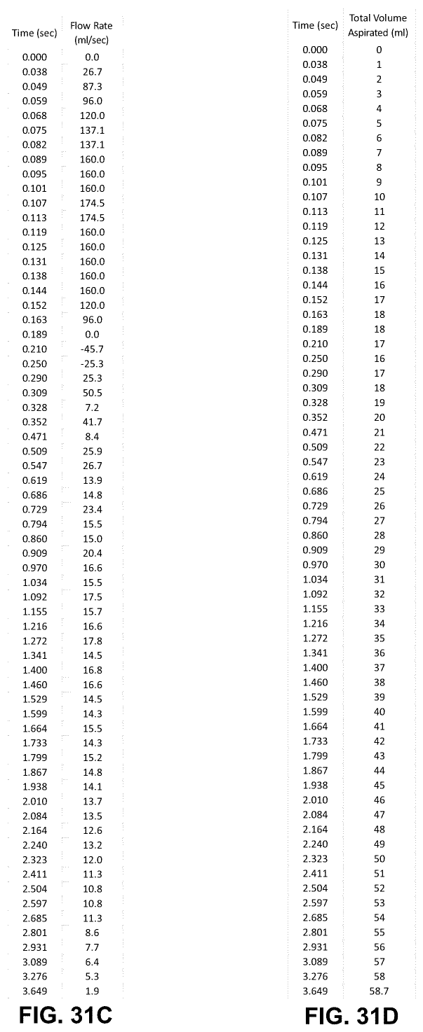

FIGS. 31A and 31B show the results of another experiment performed on an embodiment of an aspiration catheter as disclosed herein and using a 60 cc syringe as the suction source, according to the setup details and procedure described above. The aspiration catheter is a 24 Fr catheter. The 60 cc syringe is coupled directly with the aspiration tubing 618.

FIG. 31A shows the flow rate in ml per second over time. With reference to FIG. 31A, the catheter has a flow rate of 0 ml per second at the beginning of the experiment when the aspiration control valve is in the off or closed position. This corresponds to time zero on the horizontal scale. The flow rate of the water through the catheter then quickly increases to the first flow rate range. In the embodiment of the 24 Fr aspiration catheter tested in Experiment 3, the maximum flow rate measured was 174.5 ml per second and the first flow rate range measured ranged from 160.0 ml per second to 174.5 ml per second over a 0.055 second interval, starting at 0.089 seconds and lasting through 0.144 seconds. The flow rate started to decrease after 0.144 seconds. The measured flow rate eventually decreased to a second flow rate below 20 ml per second after 0.909 seconds. However, it is believed that the actual flow rate was below 20 ml per second after 0.189 seconds but that the perturbations in the graduated cylinder caused the flow rate measurements to oscillate after the flow rate started to decrease.

FIG. 31B shows the total volume aspirated in ml over time (in seconds).

FIGS. 31C and 31D show the data used to generate the plots of FIGS. 31A and 31B, respectively.

### Experiment 4

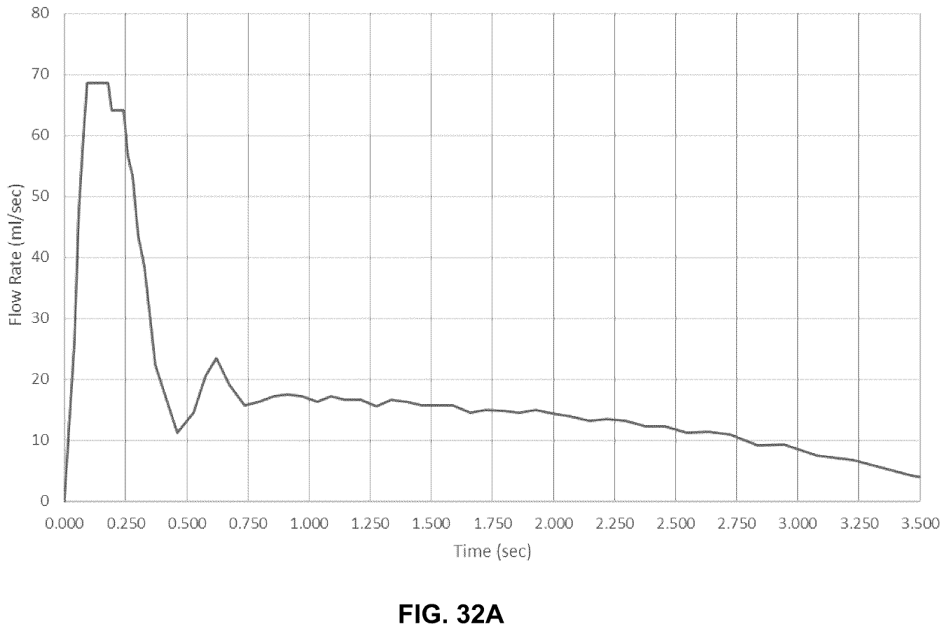

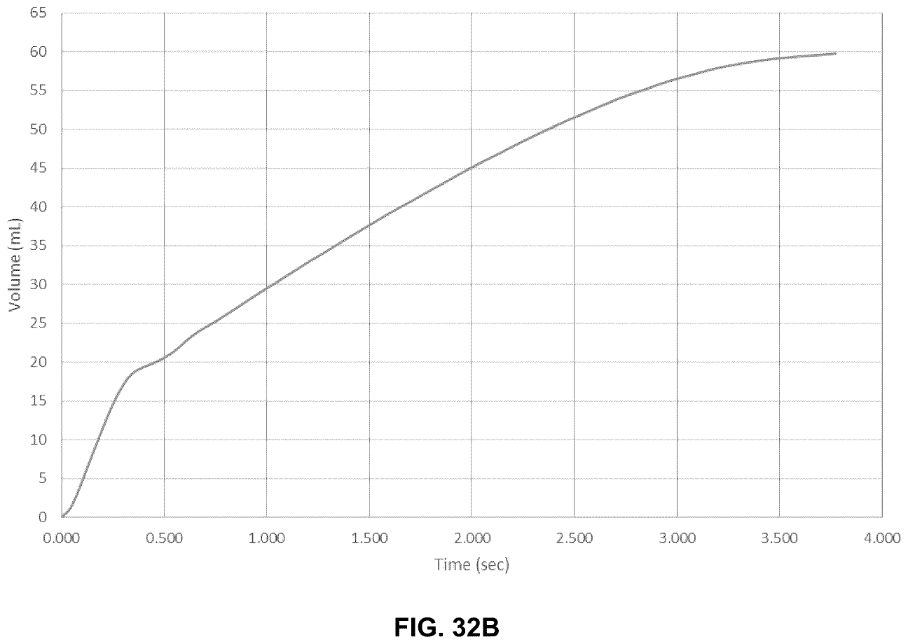

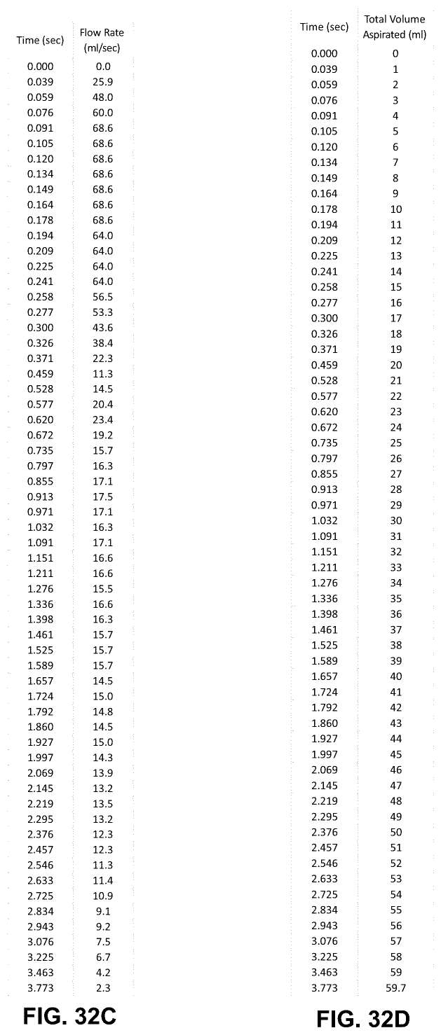

FIGS. 32A and 32B show the results of another experiment performed on an embodiment of an aspiration catheter as disclosed herein and using a 60 cc syringe as the suction source, according to the setup details and procedure described above. The aspiration catheter is a 16 Fr catheter. The 60 cc syringe is coupled directly with the aspiration tubing 618.

FIG. 32A shows the flow rate in ml per second over time. With reference to FIG. 32A, the catheter has a flow rate of 0 ml per second at the beginning of the experiment when the aspiration control valve is in the off or closed position. This corresponds to time zero on the horizontal scale. The flow rate of the water through the catheter then quickly increases to the first flow rate range. In the embodiment of the 16 Fr aspiration catheter tested in Experiment 4, the maximum flow rate measured was 68.6 ml per second and the first flow rate range measured ranged from 64.0 ml per second to 68.6 ml per second over a 0.150 second interval, starting at 0.091 seconds and lasting through 0.241 seconds, though the measured flow rate was 60.0 ml per second at 0.076 seconds. The flow rate started to decrease after 0.150 seconds. The measured flow rate eventually decreased to a second flow rate below 18 ml per second after 0.371 seconds, with the exception of one spike in the measured flow rate to a value of 23.4 ml per second at 0.620 seconds. However, it is believed that the actual flow rate was below 20 ml per second after 0.189 seconds but that the perturbations in the graduated cylinder caused the flow rate measurements to oscillate after the flow rate started to decrease.

FIG. 32B shows the total volume aspirated in ml over time (in seconds).

FIGS. 32C and 32D show the data used to generate the plots of FIGS. 32A and 32B, respectively.

### Blood Analog Experiments:

The procedure for the following two experiments (i.e., Experiment 5 and Experiment 6) was the same as for Experiments 1-4, except that the graduated cylinder was filled with 100 ml of a blood analog instead of water and the same blood analog was used to prime the catheter. Note that the blood analog used in Experiments 5 and 6 consisted of approximately 49.96% glycerin, approximately 49.96% water, and approximately 0.075% xantham gum.

When the desired amount of liquid has been aspirated through the catheter, closing the aspiration control valve and turning off the suction source, in any desired order. Note that moving the aspiration control valve to the open position entails moving the lever of the aspiration control valve to the open position quickly, in some embodiments.

### Experiment 5

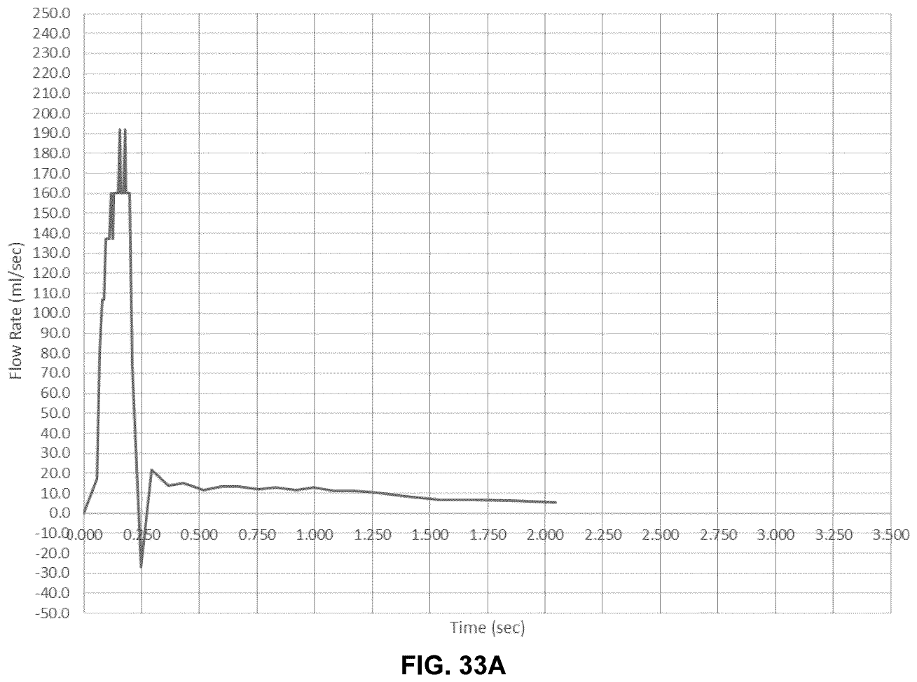

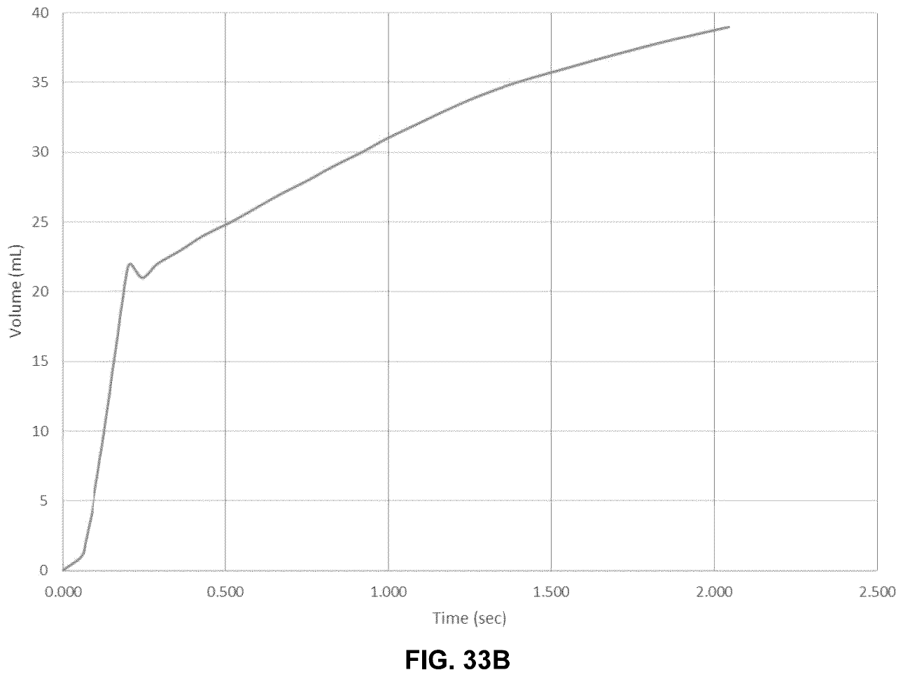

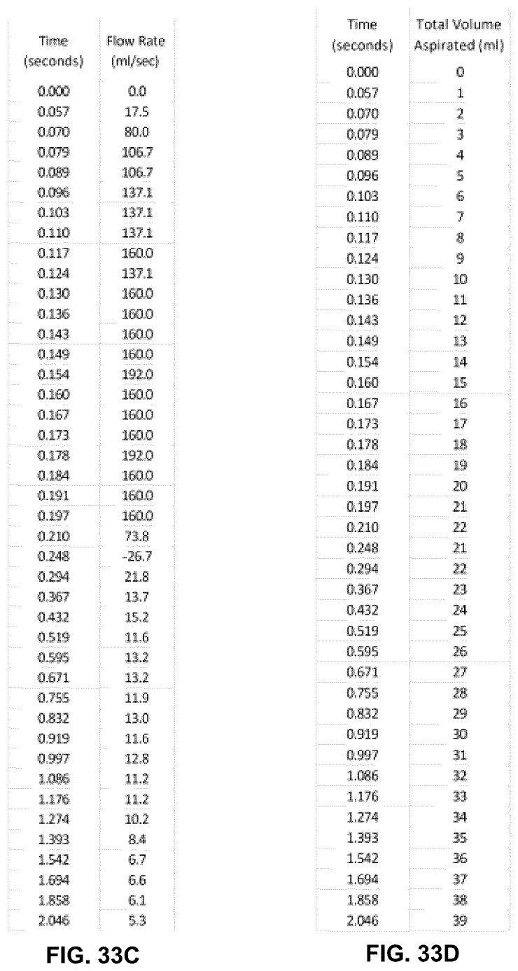

FIGS. 33A and 33B show the results of Experiment 5, performed on an embodiment of an aspiration catheter as disclosed herein and using an Imperative Care pump as the suction source, according to the setup details and procedure described above. The aspiration catheter is a 24 Fr catheter and the fluid is a blood analog. The Imperative Care pump provides suction to the catheter through a tube having a length that is approximately 106 inches (for example, without limitation, 106 inches) and that has the same inner diameter as aspiration tubing 618.

FIG. 33A shows the flow rate of the blood analog in ml per second over time. With reference to FIG. 33A, the catheter has a flow rate of 0 ml per second at the beginning of the experiment when the aspiration control valve is in the off or closed position. This corresponds to time zero on the horizontal scale. The flow rate of the blood analog through the catheter then quickly increases to the first flow rate range. In the embodiment of the 24 Fr aspiration catheter tested in Experiment 5, the maximum flow rate measured was 192 ml per second, the first flow rate range measured ranged from 160 ml per second at, for example and without limitation, 0.117 seconds to 192 ml per second at, for example and without limitation, 0.178 seconds, over a 0.08 second interval, starting at 0.117 seconds and lasting through 0.197 seconds. The flow rate started to decrease after 0.197 seconds. The measured flow rate eventually decreased to a second flow rate range that ranged from 5.3 ml per second to 15.2 ml per second after 0.432 seconds.

FIG. 33B shows the total volume aspirated in ml over time (in seconds).

FIGS. 33C and 33D show the data used to generate the plots of FIGS. 33A and 33B, respectively.

### Experiment 6

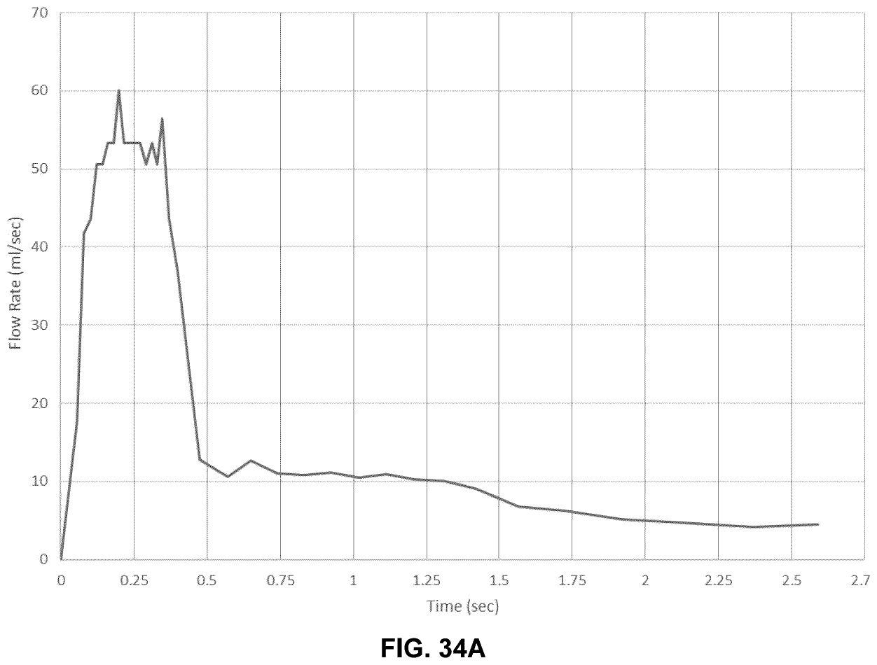

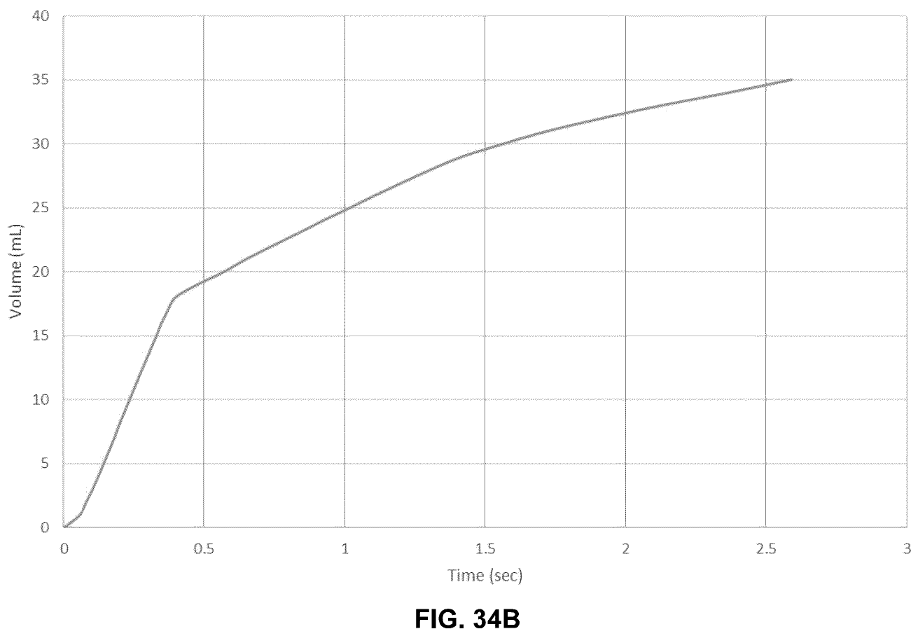

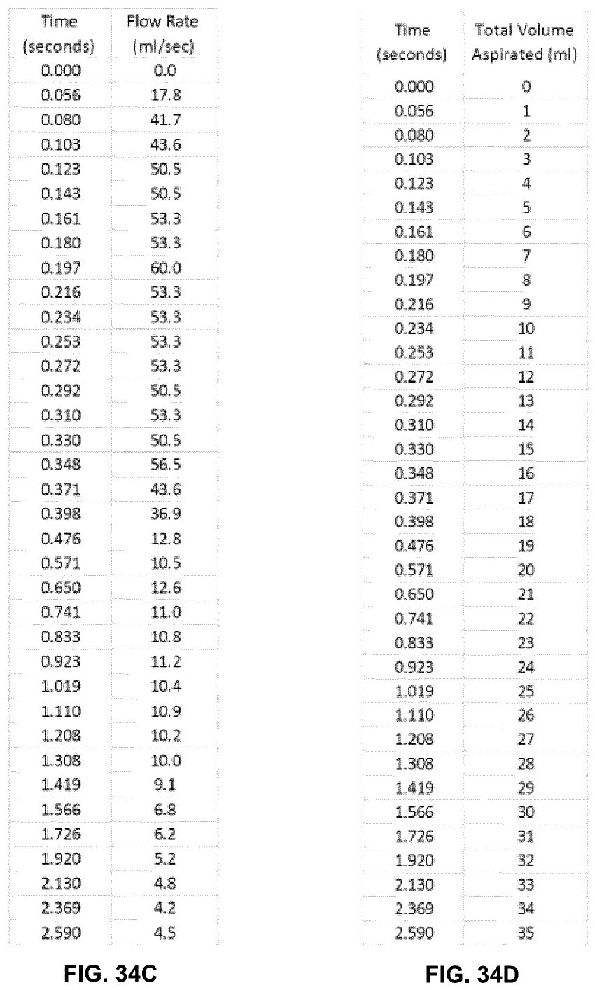

FIGS. 34A and 34B show the results of Experiment 6, performed on an embodiment of an aspiration catheter as disclosed herein and using an Imperative Care pump as the suction source, according to the setup details and procedure described above. The aspiration catheter is a 16 Fr catheter and the fluid is a blood analog. The Imperative Care pump provides suction to the catheter through a tube having a length that is approximately 106 inches (for example, without limitation, 106 inches) and that has the same inner diameter as aspiration tubing 618.

FIG. 34A shows the flow rate of the blood analog in ml per second over time. With reference to FIG. 34A, the catheter has a flow rate of 0 ml per second at the beginning of the experiment when the aspiration control valve is in the off or closed position. This corresponds to time zero on the horizontal scale. The flow rate of the blood analog through the catheter then quickly increases to the first flow rate range. In the embodiment of the 16 Fr aspiration catheter tested in Experiment 6, the maximum flow rate measured was 60 ml per second. The first flow rate range measured ranged from 50.5 ml per second at, for example and without limitation, 0.123 seconds to 60 ml per second at, for example and without limitation, 0.197 seconds, ending with a flow rate of 56.5 ml per second at 0.348 seconds, over a 0.225 second interval, starting at 0.123 seconds and lasting through 0.348 seconds. The flow rate started to decrease after 0.348 seconds. The measured flow rate eventually decreased to a second flow rate range that ranged from 4.2 ml per second to 12.8 ml per second after 0.476 seconds.

FIG. 34B shows the total volume aspirated in ml over time (in seconds).

FIGS. 34C and 34D show the data used to generate the plots of FIGS. 34A and 34B, respectively.

### NUMBERED EMBODIMENTS

Described below are a number of non-limiting, example embodiments encompassed by this application.

Embodiment 1: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that:
      a flow rate of a fluid through the fluid flow path upstream of the aspiration control valve increases to a first flow rate range that is greater than zero;
      while the aspiration control valve continues to be in the open state, the flow rate of the fluid through the fluid flow path is maintained at the first flow rate range for a first period of time;
      while the aspiration control valve continues to be in the open state, without any substantial change to the suction pressure provided by the source of suction, after the first period of time, the flow rate of the fluid through the fluid flow path drops to a second flow rate range that is greater than zero and that is less than the first flow rate range.

Embodiment 2: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that:
      a flow rate of a fluid through the fluid flow path increases to a first flow rate range that is greater than zero;
      while the aspiration control valve continues to be in the open state, the flow rate of the fluid through the fluid flow path is maintained at the first flow rate range until a first volume of the fluid has been aspirated through the catheter sheath; and
      while the aspiration control valve continues to be in the open state, after the first volume of the fluid has been aspirated through the catheter sheath, the flow rate of the fluid through the fluid flow path drops to a second flow rate range that is greater than zero but less than the first flow rate range.

Embodiment 3: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that a peak flow rate of a fluid through the fluid flow path is achieved within 0.08 seconds after suction is provided to the fluid flow path.

Embodiment 4: The vacuum aspiration system of any one of Embodiments 1-3, wherein the first flow rate range is greater than 100 ml per second.

Embodiment 5: The vacuum aspiration system of any one of Embodiments 1-3, wherein the first flow rate range is greater than 150 ml per second.

Embodiment 6: The vacuum aspiration system of any one of Embodiments 1-3, wherein the first flow rate range is greater than 180 ml per second, or greater than 190 ml per second.

Embodiment 7: The vacuum aspiration system of any one of Embodiments 1-3, wherein a total volume of the fluid aspirated through the catheter sheath is less than 60 ml when the flow rate of the fluid through the fluid flow path drops to the second flow rate range.

Embodiment 8: The vacuum aspiration system of any one of Embodiments 1-7, wherein the total volume of the fluid aspirated through the catheter sheath when the flow rate of the fluid through the fluid flow path drops to the second flow rate range is less than 55 ml when the flow rate of the fluid through the fluid flow path drops to the second flow rate range.

Embodiment 9: The vacuum aspiration system of any one of Embodiments 1-8, wherein the total volume of the fluid aspirated through the catheter sheath when the flow rate of the fluid through the fluid flow path drops to the second flow rate range is less than 50 ml when the flow rate of the fluid through the fluid flow path drops to the second flow rate range.

Embodiment 10: The vacuum aspiration system of any one of Embodiments 1-9, wherein the first period of time is from 0.1 seconds to 0.3 seconds.

Embodiment 11: The vacuum aspiration system of any one of Embodiments 1-9, wherein the first period of time is from 0.1 seconds to 0.25 seconds.

Embodiment 12: The vacuum aspiration system of any one of Embodiments 1-9, wherein the first period of time is from 0.1 seconds to 0.2 seconds.

Embodiment 13: The vacuum aspiration system of any one of Embodiments 1-12, wherein the second flow rate range is less than half of the first flow rate range.

Embodiment 14: The vacuum aspiration system of any one of Embodiments 1-12, wherein the second flow rate range is less than 25% of the first flow rate range.

Embodiment 15: The vacuum aspiration system of any one of Embodiments 1-12, wherein the second flow rate range is less than 15% of the first flow rate range.

Embodiment 16: The vacuum aspiration system of any one of Embodiments 1-12, wherein the second flow rate range is less than 10% of the first flow rate range.

Embodiment 17: The vacuum aspiration system of any one of Embodiments 1-12, wherein the second flow rate range is less than 5% of the first flow rate range.

Embodiment 18: The vacuum aspiration system of any one of Embodiments 1-12, wherein the second flow rate range is less than 30 ml per second.

Embodiment 19: The vacuum aspiration system of any one of Embodiments 1-12, wherein the second flow rate range is less than 20 ml per second.

Embodiment 20: The vacuum aspiration system of any one of Embodiments 1-12, wherein the second flow rate range is less than 10 ml per second.

Embodiment 21: The vacuum aspiration system of Embodiment 2, wherein the first volume of the fluid is 16 ml or approximately 16 ml, 17.5 ml or approximately 17.5 ml, 18 ml or approximately 18 ml, 19 ml or approximately 19 ml, 20 ml or approximately 20 ml, or from 10 ml or approximately 10 ml to 30 ml or approximately 30 ml, or from 15 ml or approximately 15 ml to 25 ml or approximately 25 ml, or of any value, approximate value, or range of values in any of the foregoing ranges.

Embodiment 22: The vacuum aspiration system of any one of Embodiments 1-21, comprising the source of suction.

Embodiment 23: The vacuum aspiration system of any one of Embodiments 1-22, wherein the source of suction applies a suction pressure of 28 inHg or approximately 28 inHg, or from 26 inHg to 29.92 inHg.

Embodiment 24: The vacuum aspiration system of any one of Embodiments 1-23, wherein the source of suction is a vacuum pump.

Embodiment 25: The vacuum aspiration system of any one of Embodiments 1-24, wherein the source of suction is a syringe.

Embodiment 26: The vacuum aspiration system of any one of Embodiments 1-25, further comprising a suction conduit 618 in communication with the fluid flow path and configured to be fluidically coupled with the source of suction.

Embodiment 27: The vacuum aspiration system of any one of Embodiments 1-26, wherein the aspiration control valve is moved from the closed state to the open state rapidly enough to not affect the flow rate of the fluid through the flow path.

Embodiment 28: The vacuum aspiration system of any one of Embodiments 1-27, wherein the vacuum aspiration system is configured to provide a rapid burst of suction through the catheter so that the catheter reaches a peak flow rate of a fluid through the fluid flow path within 0.06 seconds, wherein the fluid is water.

Embodiment 29: The vacuum aspiration system of any one of Embodiments 1-27, wherein the vacuum aspiration system is configured to provide a rapid burst of suction through the catheter so that the catheter reaches a peak flow rate of a fluid through the fluid flow path at approximately 0.055 seconds, wherein the fluid is water.

Embodiment 30: The vacuum aspiration system of any one of Embodiments 1-27, wherein the vacuum aspiration system is configured to provide a rapid burst of suction through the catheter so that the catheter reaches a peak flow rate of a fluid through the fluid flow path within 0.03 seconds to 0.06 seconds, wherein the fluid is water.

Embodiment 31: The vacuum aspiration system of any one of Embodiments 1-30, wherein the fluid is water.

Embodiment 32: The vacuum aspiration system of any one of Embodiments 1-31, wherein the fluid flow path catheter is primed with the fluid before suction is applied to the catheter.

Embodiment 33: The vacuum aspiration system of any one of Embodiments 1-32, wherein the fluid is water, and the peak flow rate of the water through the fluid flow path is 183 ml per second.

Embodiment 34: The vacuum aspiration system of any one of Embodiments 1-32, wherein the fluid is water, and the peak flow rate of the water through the fluid flow path is from 173 ml per second to 193 ml per second.

Embodiment 35: The vacuum aspiration system of any one of Embodiments 1-32, wherein the fluid is water, and the peak flow rate of the water through the fluid flow path is greater than 180 ml per second.

Embodiment 36: The vacuum aspiration system of any one of Embodiments 1-35, wherein the vacuum aspiration system is configured so that the peak flow rate of the fluid through the catheter is sustained for no more than 0.3 seconds.

Embodiment 37: The vacuum aspiration system of any one of Embodiments 1-36, wherein the vacuum aspiration system is configured so that the peak flow rate of the fluid through the catheter is sustained for no more than 0.2 seconds.

Embodiment 38: The vacuum aspiration system of any one of Embodiments 1-35, wherein the vacuum aspiration system is configured so that the peak flow rate of the fluid through the catheter is sustained for no more than 0.15 seconds.

Embodiment 39: The vacuum aspiration system of any one of Embodiments 1-38, wherein the vacuum aspiration system is configured to provide a rapid drop-off of the suction provided through the catheter such that the flow rate of the fluid through the fluid flow path drops below 20 mL per second within 0.4 seconds after the flow rate of the fluid through the fluid flow path first reaches the peak flow rate while the aspiration control valve remains in an open state.

Embodiment 40: The vacuum aspiration system of any one of Embodiments 1-38, wherein the vacuum aspiration system is configured to provide a rapid drop-off of the suction provided through the catheter such that the flow rate of the fluid through the fluid flow path drops below 10 mL per second within 0.3 seconds after the flow rate of the fluid through the fluid flow path first reaches the peak flow rate while the aspiration control valve remains in an open state.

Embodiment 41: The vacuum aspiration system of any one of Embodiments 1-38, wherein the vacuum aspiration system is configured to provide a rapid drop-off of the suction provided through the catheter such that the flow rate of the fluid through the fluid flow path drops below 10 mL per second within 0.2 seconds after the flow rate of the fluid through the fluid flow path first reaches the peak flow rate while the aspiration control valve remains in an open state.

Embodiment 42: The vacuum aspiration system of Embodiment 3, wherein the aspiration control valve is configured to move between a first position wherein the aspiration control valve is closed and a second position wherein the aspiration control valve is open, and wherein suction is provided to the fluid flow path by moving the aspiration control valve to the second position, thereby providing the suction from the source of suction to the fluid flow path.

Embodiment 43: The vacuum aspiration system of any one of Embodiments 1-42, wherein the aspiration control valve comprises a lever.

Embodiment 44: The vacuum aspiration system of any one of Embodiments 1-43, further comprising a clot container coupled with the housing, the clot container being in fluid communication with the fluid flow path.

Embodiment 45: The vacuum aspiration system of Embodiment 44, wherein the clot container has a filter therein.

Embodiment 46: The vacuum aspiration system of any one of Embodiments 1-45, wherein the fluid flow path passes through a suction conduit 618 that fluidically couples to the source of suction, wherein the suction conduit 618 has an internal diameter (D8) of 2.67 mm, or approximately 2.67 mm, or from 2 mm or approximately 2 mm to 4 mm or approximately 4 mm, or from 2.5 mm or approximately 2.5 mm to 3 mm or approximately 3 mm, or of any value, approximate value, or range of values in any of the foregoing ranges.

Embodiment 47: The vacuum aspiration system of any one of Embodiments 1-45, further comprising a clot container coupled with the housing, the clot container having an internal space, an inlet, and an outlet, wherein the outlet has a conduit that is in fluid communication with the fluid flow path.

Embodiment 48: The vacuum aspiration system of Embodiment 47, wherein the conduit of the outlet of the clot container has a minimum internal diameter (D6) of 2 mm, or approximately 2 mm, or from 1 mm or approximately 1 mm to 3 mm or approximately 3 mm, or from 1.5 mm or approximately 1.5 mm to 2.5 mm or approximately 2.5 mm, or of any value, approximate value, or range of values in any of the foregoing ranges.

Embodiment 49: The vacuum aspiration system of Embodiment 47, wherein the conduit of the outlet of the clot container has a first internal diameter (D6) of 2 mm, or approximately 2 mm, or from 1 mm or approximately 1 mm to 3 mm or approximately 3 mm, or from 1.5 mm or approximately 1.5 mm to 2.5 mm or approximately 2.5 mm, or of any value, approximate value, or range of values in any of the foregoing ranges, and a second internal diameter (D7) of 2.25 mm, or approximately 2.25 mm, or from 1.5 mm or approximately 1.5 mm to 3 mm or approximately 3 mm, or from 2 mm or approximately 2 mm to 2.5 mm or approximately 2.5 mm, or of any value, approximate value, or range of values in any of the foregoing ranges.

Embodiment 50: The vacuum aspiration system of any one of Embodiments 47-49, wherein the outlet is downstream of the inlet.

Embodiment 51: The vacuum aspiration system of any one of Embodiments 46-49, comprising a conduit extending from the aspiration control valve to the inlet of the clot container having an internal diameter (D5) of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges.

Embodiment 52: The vacuum aspiration system of any one of Embodiments 1-51, comprising a conduit extending from the aspiration control valve upstream of the aspiration control valve toward a proximal end of the catheter sheath, the conduit extending from the aspiration control valve having an internal diameter (D3, D4) through the conduit of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges.

Embodiment 53: The vacuum aspiration system of Embodiment 52, comprising a sealing element at a proximal end of the catheter sheath, the sealing element having a passageway therethrough that is part of the fluid flow path and that has a minimum internal diameter (D1) of 4.67 mm, or approximately 4.67 mm, or from 3.7 mm or approximately 3.7 mm to 5.7 mm or approximately 5.7 mm, or from 4.2 mm or approximately 4.2 mm to 5.2 mm or approximately 5.2 mm, or of any value, approximate value, or range of values in any of the foregoing ranges.

Embodiment 54: The vacuum aspiration system of Embodiment 53, wherein the sealing element has a first end that is distal to a second end, the first end being upstream of the first end in the fluid pathway, wherein the minimum internal diameter (D1) is at the first end and the second end has an internal diameter (D2) of 5.14 mm, or approximately 5.14 mm, or from 4 mm or approximately 4 mm to 6.5 mm or approximately 6.5 mm, or from 4.5 mm or approximately 4.5 mm to 6 mm or approximately 6 mm, or of any value, approximate value, or range of values in any of the foregoing ranges.

Embodiment 55: The vacuum aspiration system of any one of Embodiments 1-54, further comprising a clot container coupled with the housing, an inlet into the clot container having an internal diameter of 7 mm, or approximately 7 mm, or from 5 mm or approximately 5 mm to 9 mm or approximately 9 mm, or from 6 mm or approximately 6 mm to 8 mm or approximately 8 mm, or of any value, approximate value, or range of values in any of the foregoing ranges, and a an outlet from the clot container downstream of the inlet into the clot container, the outlet having an internal diameter of 2.8 mm, or approximately 2.8 mm, or from 1.8 mm or approximately 1.8 mm to 3.8 mm or approximately 3.8 mm, or from 2.5 mm or approximately 2.5 mm to 3.1 mm or approximately 3.1 mm, or of any value, approximate value, or range of values in any of the foregoing ranges.

Embodiment 56: The vacuum aspiration system of any one of Embodiments 1-55, wherein the flow rate drops from the first flow rate to the second flow rate in 0.05 seconds, approximately 0.05 seconds, or less than 0.05 seconds, or in 0.075 seconds, approximately 0.075 seconds, or less than 0.075 seconds, or from 0.025 seconds or approximately 0.025 seconds to 0.075 seconds or approximately 0.075 seconds, or from 0.04 seconds or approximately 0.04 seconds to 0.06 seconds or approximately 0.06 seconds, or of any value, approximate value, or range of values in any of the foregoing ranges.

Embodiment 57: The vacuum aspiration system of any one of Embodiments 1-43 or 52-56, further comprising a clot container coupled with the housing, wherein the flow rate drops from the first flow rate to the second flow rate when the clot container becomes full with the fluid that has been aspirated through the catheter sheath.

Embodiment 58: The vacuum aspiration system of Embodiment 57, wherein the clot container has a volume of 17.7 ml or approximately 17.7 ml, or 40 ml or approximately 40 ml, or 60 ml or approximately 60 ml, or from 15 ml or approximately 15 ml to 20 ml or approximately 20 ml, or from 17 ml or approximately 17 ml to 19 ml or approximately 19 ml, or of any value, approximate value, or range of values in any of the foregoing ranges.

Embodiment 59: The vacuum aspiration system of any one of Embodiments 57-58, wherein the first period of time is increased by increasing the volume of the clot container and/or a volume of the fluid flow path upstream of the clot container.

Embodiment 60: The vacuum aspiration system of any one of Embodiments 1-59, wherein dropping the flow rate of fluid through the fluid flow path to the second flow rate after the first period of time reduces (e.g., substantially reduces) a loss of blood from a patient during a thrombectomy procedure.

Embodiment 61: The vacuum aspiration system of any one of Embodiments 1-60, wherein the aspiration catheter assembly is a 24 Fr aspiration catheter.

Embodiment 62: The vacuum aspiration system of any one of Embodiments 1-60, wherein the aspiration catheter assembly is a 16 Fr aspiration catheter.

Embodiment 63: The vacuum aspiration system of any one of Embodiments 1-62, wherein the fluid is at room temperature.

Embodiment 64: The vacuum aspiration system of any one of Embodiments 1-62, wherein the fluid is at a temperature between 68 degrees F. and 73 degrees F.

Embodiment 65: The vacuum aspiration system of any one of Embodiments 1-64, wherein the fluid is homogeneous (e.g., does not have any clots, thicker, or more viscous substances therein).

Embodiment 66: The vacuum aspiration system of any one of Embodiments 1-64, wherein a volume of the fluid flow path downstream of the catheter sheath and downstream to and including the clot container is 22.6 ml or approximately 22.6 ml, or from 18 ml or approximately 18 ml to 25 ml or approximately 25 ml, or from 20 ml or approximately 20 ml to 25 ml or approximately 25 ml, or of any value, approximate value, or range of values in any of the foregoing ranges.

Embodiment 67: The vacuum aspiration system of any one of Embodiments 1-65, further comprising a suction conduit 618 in communication with the fluid flow path and configured to be fluidically coupled with the source of suction, wherein a volume of the fluid pathway from and including the suction conduit 618 upstream up to the clot container is 1.8 ml or approximately 1.8 ml, or from 1.5 ml or approximately 1.5 ml to 2.0 ml or approximately 2.0 ml, or from 1.7 ml or approximately 1.7 ml to 1.9 ml or approximately 1.9 ml, or of any value, approximate value, or range of values in any of the foregoing ranges.

Embodiment 68: A method of aspirating a fluid through a catheter of an aspiration system, comprising:
positioning an aspiration control valve of the catheter in a closed position;
with the aspiration control valve in the closed position and a fluid flow path of the catheter in fluid communication with the fluid, applying a suction pressure to a fluid flow path of the catheter;
moving the aspiration control valve to an open position to aspirate the fluid through the fluid flow path of the catheter, wherein a flow rate of the fluid through the fluid flow path increases to a first flow rate range that is greater than 50 ml per second; and
when the flow rate of the fluid through the fluid flow path decreases below a second value, moving the aspiration control valve back to the closed position;
wherein:
   the catheter is configured to decrease the flow rate of the fluid through the fluid flow path without changing the suction pressure being applied to the fluid flow path of the catheter.

Embodiment 69: The method of Embodiment 68, wherein the catheter is configured to decrease the flow rate of the fluid through the fluid flow path to the second value of the flow rate range without making any changes to the aspiration system.

Embodiment 70: The method of any one of Embodiments 68 or 71-74, wherein the first flow rate range is greater than 100 ml per second or approximately 100 ml per second, or is greater than 150 ml per second or approximately 150 ml per second, or is greater than 180 ml per second or approximately 180 ml per second, or is greater than 190 ml per second or approximately 190 ml per second, or is greater than 200 ml per second or approximately 200 ml per second, or is from 100 ml per second or is approximately 100 ml per second to 200 ml per second or approximately 200 ml per second, or is from 150 ml per second or approximately 150 ml per second to 200 ml per second or approximately 200 ml per second, or of any value, approximate value, or range of values in any of the foregoing ranges.

Embodiment 71: The method of Embodiment 68, wherein the second value is between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges.

Embodiment 72: A method of aspirating a clot material during a thrombectomy procedure, comprising:
positioning an aspiration control valve of an aspiration catheter in a closed position;
applying a suction pressure to a fluid flow path of the catheter;
positioning an aspiration catheter within a predetermined distance of a clot within a patient's vasculature;
moving the aspiration control valve to an open position to aspirate the clot through the fluid flow path of the aspiration catheter, wherein a flow rate of a fluid through the fluid flow path upstream of the aspiration control valve increases to a first flow rate range that is greater than zero;
when the flow rate of the fluid through the fluid flow path decreases below a second flow rate value, moving the aspiration control valve back to the closed position;
after the flow rate of the fluid through the fluid flow path has decreased below the second flow rate value and after moving the aspiration control valve back to the closed position, withdrawing the aspiration catheter a predetermined distance; and
after withdrawing the aspiration catheter the predetermined distance, moving the aspiration control valve again to the open position to continue to aspirate the clot through the fluid flow path of the aspiration catheter, wherein the flow rate of the fluid through the fluid flow path upstream of the aspiration control valve increases again to the first flow rate range.

Embodiment 73: The method of Embodiment 71, further comprising, when the flow rate of the fluid through the fluid flow path again decreases below the second flow rate value, moving the aspiration control valve back to the closed position.

The method of Embodiment 72, further comprising,
withdrawing the aspiration catheter a second predetermined distance; and
after withdrawing the aspiration catheter the second predetermined distance, moving the aspiration control valve again to the open position to continue to aspirate the clot through the fluid flow path of the aspiration catheter, wherein the flow rate of the fluid through the fluid flow path upstream of the aspiration control valve increases again to the first flow rate range.

Embodiment 74: The method of Embodiment 73, further comprising, when the flow rate of the fluid through the fluid flow path decreases below the second flow rate value for a third time, moving the aspiration control valve back to the closed position.

Embodiment 75: The method of any one of Embodiments 71-74, wherein the first flow rate range is greater than 40 ml per second, when the fluid is blood.

Embodiment 76: The method of any one of Embodiments 71-74, wherein the first flow rate range is greater than 50 ml per second, when the fluid is blood.

Embodiment 77: The method of any one of Embodiments 71-74, wherein the first flow rate range is greater than 60 ml per second, when the fluid is blood.

Embodiment 78: The method of any one of Embodiments 71-74, wherein the first flow rate range is from 40 ml per second to 120 ml per second, when the fluid is blood.

Embodiment 79: The method of any one of Embodiments 71-78, wherein the second flow rate value is from 50 ml per second to 100 ml per second, when the fluid is blood.

Embodiment 80: The method of any one of Embodiments 71-78, wherein the second flow rate value is between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges.

Embodiment 81: The method of any one of Embodiments 71-80, wherein the fluid is blood.

Embodiment 82: The method of any one of Embodiments 71-81, wherein the flow rate of the fluid through the fluid flow path decreases below the second flow rate value when a clot container of the aspiration catheter becomes full with the fluid or the fluid and the clot material.

Embodiment 83: The method of any one of Embodiments 71-81, wherein the aspiration catheter is configured to decrease the flow rate of the fluid through the fluid flow path below the second flow rate value without any user input.

### Additional Numbered Embodiments

Described below are a number of additional non-limiting, example embodiments encompassed by this application.

Embodiment 1: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that:
      a flow rate of a fluid through the fluid flow path upstream of the aspiration control valve increases to a first flow rate range that is greater than zero;
      while the aspiration control valve continues to be in the open state, the flow rate of the fluid through the fluid flow path is maintained at the first flow rate range for a first period of time;
      while the aspiration control valve continues to be in the open state, without any substantial change to the suction pressure provided by the source of suction, after the first period of time, the flow rate of the fluid through the fluid flow path automatically drops to a second flow rate range that is greater than zero and that is less than the first flow rate range.

Embodiment 2: The vacuum aspiration system of any one of the previous Embodiments, wherein the first flow rate range is greater than 100 ml per second.

Embodiment 3: The vacuum aspiration system of any one of the previous Embodiments, wherein the first flow rate range is greater than 150 ml per second.

Embodiment 4: The vacuum aspiration system of any one of the previous Embodiments, wherein the first flow rate range is at least 180 ml per second when the fluid being aspirated is water, the source of suction is a pump that provides at least a −28 inHg suction pressure, and the catheter sheath is a 24 Fr or larger catheter sheath.

Embodiment 5: The vacuum aspiration system of any one of the previous Embodiments, wherein the first flow rate range is at least 160 ml per second when the fluid being aspirated is water, the source of suction is a 60 cc syringe, and the catheter sheath is a 24 Fr or larger catheter sheath.

Embodiment 6: The vacuum aspiration system of any one of the previous Embodiments, wherein the first flow rate range is at least 80 ml per second when the fluid being aspirated is water, the source of suction is a pump that provides at least a −28 inHg suction pressure, and the catheter sheath is a 16 Fr or larger catheter sheath.

Embodiment 7: The vacuum aspiration system of any one of the previous Embodiments, wherein the first flow rate range is at least 60 ml per second when the fluid being aspirated is water, the source of suction is a 60 cc syringe, and the catheter sheath is a 16 Fr or larger catheter sheath.

Embodiment 8: The vacuum aspiration system of any one of the previous Embodiments, wherein a substantial change would be more than a 10% change in the suction pressure provided by the source of suction.

Embodiment 9: The vacuum aspiration system of any one of the previous Embodiments, wherein a total volume of the fluid aspirated through the catheter sheath is less than 60 ml (or less than 55 ml) when the flow rate of the fluid through the fluid flow path automatically drops to the second flow rate range.

Embodiment 10: The vacuum aspiration system of any one of the previous Embodiments, wherein a total volume of the fluid aspirated through the catheter sheath is less than 25 ml when the flow rate of the fluid through the fluid flow path automatically drops to the second flow rate range.

Embodiment 11: The vacuum aspiration system of any one of the previous Embodiments, wherein the first period of time is at least 0.1 seconds or approximately 0.1 seconds and is less than 0.3 seconds or approximately 0.3 seconds.

Embodiment 12: The vacuum aspiration system of any one of the previous Embodiments, wherein the first period of time is at least 0.1 seconds or approximately 0.1 seconds and is less than 0.25 seconds or approximately 0.25 seconds.

Embodiment 13: The vacuum aspiration system of any one of the previous Embodiments, wherein the first period of time is at least 0.1 seconds or approximately 0.1 seconds and is less than 0.2 seconds or approximately 0.2 seconds.

Embodiment 14: The vacuum aspiration system of any one of the previous Embodiments, wherein the second flow rate range is less than half of the first flow rate range.

Embodiment 15: The vacuum aspiration system of any one of the previous Embodiments, wherein the second flow rate range is less than 15% of the first flow rate range.

Embodiment 16: The vacuum aspiration system of any one of the previous Embodiments, wherein the second flow rate range is less than 20 ml per second.

Embodiment 17: The vacuum aspiration system of any one of the previous Embodiments, wherein the second flow rate range is less than 10 ml per second.

Embodiment 18: The vacuum aspiration system of any one of the previous Embodiments, comprising the source of suction.

Embodiment 19: The vacuum aspiration system of any one of the previous Embodiments, wherein the source of suction applies a suction pressure of at least −28 inHg or at least approximately −28 inHg.

Embodiment 20: The vacuum aspiration system of any one of the previous Embodiments, wherein the source of suction is a vacuum pump.

Embodiment 21: The vacuum aspiration system of any one of the previous Embodiments, wherein the source of suction is a 60 cc syringe.

Embodiment 22: The vacuum aspiration system of any one of the previous Embodiments, further comprising a suction conduit in communication with the fluid flow path and configured to be fluidically couplable with the source of suction.

Embodiment 23: The vacuum aspiration system of any one of the previous Embodiments, wherein the aspiration control valve is moved from the closed state to the open state rapidly so that the suction pressure is provided rapidly to the fluid flow path upstream of the aspiration control valve.

Embodiment 24: The vacuum aspiration system of any one of the previous Embodiments, wherein the vacuum aspiration system is configured to provide a rapid burst of suction through the catheter when the aspiration control valve is moved to the open state so that the catheter reaches a flow rate of at least 190 ml per second in less than 0.09 seconds, when the fluid is water, the source of suction is a pump that provides at least a −28 inHg suction pressure, and the catheter sheath is a 24 Fr or larger catheter sheath.

Embodiment 25: The vacuum aspiration system of any one of the previous Embodiments, wherein the vacuum aspiration system is configured to provide a rapid burst of suction through the catheter when the aspiration control valve is moved to the open state so that the catheter reaches a flow rate of at least 70 ml per second in less than 0.09 seconds, when the fluid is water, the source of suction is a pump that provides at least a −28 inHg suction pressure, and the catheter sheath is a 16 Fr or larger catheter sheath.

Embodiment 26: The vacuum aspiration system of any one of the previous Embodiments, wherein the vacuum aspiration system is configured to provide a rapid burst of suction through the catheter when the aspiration control valve is moved to the open state so that the catheter reaches a flow rate of at least 160 ml per second in less than 0.09 seconds, when the fluid is water, the source of suction is a 60 cc syringe, and the catheter sheath is a 24 Fr or larger catheter sheath.

Embodiment 27: The vacuum aspiration system of any one of the previous Embodiments, wherein the vacuum aspiration system is configured to provide a rapid burst of suction through the catheter when the aspiration control valve is moved to the open state so that the catheter reaches a flow rate of at least 60 ml per second in less than 0.08 seconds, when the fluid is water, the source of suction is a 60 cc syringe, and the catheter sheath is a 16 Fr or larger catheter sheath.

Embodiment 28: The vacuum aspiration system of any one of the previous Embodiments, wherein the vacuum aspiration system is configured to provide a rapid burst of suction through the catheter when the aspiration control valve is moved to the open state so that the catheter reaches a flow rate of at least 160 ml per second in less than 0.12 seconds, when the fluid is a blood analog, the source of suction is a pump that provides at least a −28 inHg suction pressure, and the catheter sheath is a 24 Fr or larger catheter sheath.

Embodiment 29: The vacuum aspiration system of any one of the previous Embodiments, wherein the vacuum aspiration system is configured to provide a rapid burst of suction through the catheter when the aspiration control valve is moved to the open state so that the catheter reaches a flow rate of at least 50 ml per second in less than 0.13 seconds, when the fluid is a blood analog, the source of suction is a pump that provides at least a −28 inHg suction pressure, and the catheter sheath is a 16 Fr or larger catheter sheath.

Embodiment 30: The vacuum aspiration system of any one of the previous Embodiments, wherein a portion of the fluid flow path of the catheter upstream of the aspiration control valve is primed with the fluid before the aspiration control valve is moved to the open state.

Embodiment 31: The vacuum aspiration system of any one of the previous Embodiments, wherein the fluid is blood.

Embodiment 32: The vacuum aspiration system of any one of the previous Embodiments, wherein the fluid is a blood analog.

Embodiment 33: The vacuum aspiration system of any one of the previous Embodiments, wherein the fluid is water.

Embodiment 34: The vacuum aspiration system of Embodiment 34, wherein the fluid is water, and a peak flow rate of the water through the fluid flow path is at least 183 ml per second.

Embodiment 35: The vacuum aspiration system of any one of the previous Embodiments, wherein the aspiration catheter assembly is configured to provide an average flow rate of at least 120 ml per second during a period of time from when the aspiration control valve is moved to the open state to when the flow rate drops to the second flow rate range, when the fluid is water, the source of suction is a pump that provides at least a −28 inHg suction pressure, and the catheter sheath is a 24 Fr or larger catheter sheath.

Embodiment 36: The vacuum aspiration system of any one of the previous Embodiments, wherein the aspiration catheter assembly is configured to provide an average flow rate of at least 65 ml per second during a period of time from when the aspiration control valve is moved to the open state to when the flow rate drops to the second flow rate range, when the fluid is water, the source of suction is a pump that provides at least a −28 inHg suction pressure, and the catheter sheath is a 16 Fr or larger catheter sheath.

Embodiment 37: The vacuum aspiration system of any one of the previous Embodiments, wherein the vacuum aspiration system is configured so that the first flow rate range of the fluid through the catheter is sustained for no more than 0.2 seconds.

Embodiment 38: The vacuum aspiration system of any one of the previous Embodiments, wherein the vacuum aspiration system is configured so that the first flow rate range of the fluid through the catheter is sustained for no more than 0.25 seconds.

Embodiment 39: The vacuum aspiration system of any one of the previous Embodiments, wherein the vacuum aspiration system is configured so that the first flow rate range of the fluid through the catheter is sustained for no more than 0.15 seconds.

Embodiment 40: The vacuum aspiration system of any one of the previous Embodiments, wherein the vacuum aspiration system is configured to provide a rapid drop-off of the suction provided through the catheter such that the flow rate of the fluid through the fluid flow path automatically drops below 20 mL per second within 0.25 seconds after the aspiration control valve is moved to the open state, with the aspiration control valve remaining in an open state, when the fluid is water, blood, or a blood analog.

Embodiment 41: The vacuum aspiration system of any one of the previous Embodiments, wherein the vacuum aspiration system is configured to provide a rapid drop-off of the suction provided through the catheter such that the flow rate of the fluid through the fluid flow path automatically drops below 30 mL per second within 0.5 seconds after the aspiration control valve is moved to the open state, with the aspiration control valve remaining in an open state, when the fluid is water, blood, or a blood analog and the source of suction is a suction pump or a syringe.

Embodiment 42: The vacuum aspiration system of any one of the previous Embodiments, wherein the vacuum aspiration system is configured to provide a rapid drop-off of the suction provided through the catheter such that the flow rate of the fluid through the fluid flow path drops below 10 mL per second within 0.3 seconds after the flow rate of the fluid through the fluid flow path first reaches the peak flow rate while the aspiration control valve remains in an open state.

Embodiment 43: The vacuum aspiration system of any one of the previous Embodiments, wherein the aspiration control valve comprises a lever.

Embodiment 44: The vacuum aspiration system of any one of the previous Embodiments, further comprising a clot container coupled with the housing, the clot container being in fluid communication with the fluid flow path.

Embodiment 45: The vacuum aspiration system of Embodiment 45, wherein the clot container has a filter therein.

Embodiment 46: The vacuum aspiration system of Embodiment 45, wherein the clot container has a volume of 17.7 ml or approximately 17.7 ml.

Embodiment 47: The vacuum aspiration system of Embodiment 45, wherein the clot container has a volume of at least 17 ml or at least approximately 17.7 ml.

Embodiment 48: The vacuum aspiration system of Embodiment 45, wherein the clot container has a volume of at least 40 ml or at least approximately 40 ml.

Embodiment 49: The vacuum aspiration system of Embodiment 45, wherein the clot container has a volume of at least 60 ml or at least approximately 60 ml.

Embodiment 50: The vacuum aspiration system of Embodiment 45, wherein the first period of time is increased by increasing the volume of the clot container and/or a volume of the fluid flow path upstream of the clot container.

Embodiment 51: The vacuum aspiration system of any one of the previous Embodiments, wherein the fluid flow path passes through a suction conduit downstream of aspiration control valve, wherein the suction conduit is in fluidic communication with the source of suction and wherein the suction conduit has an internal diameter of from 2.5 mm or approximately 2.5 mm to 2.8 mm or approximately 2.8 mm.

Embodiment 52: The vacuum aspiration system of any one of the previous Embodiments, wherein the fluid flow path passes through at least two tubes comprising the suction conduit downstream of aspiration control valve, wherein the at least two tubes are in fluidic communication with the source of suction, wherein an internal diameter of each of the at least two tubes is from 2.5 mm or approximately 2.5 mm to 2.8 mm or approximately 2.8 mm, and wherein a combined length of the at least two tubes is more than 100 inches.

Embodiment 53: The vacuum aspiration system of any one of the previous Embodiments, further comprising a clot container coupled with the housing, the clot container having an internal space, an inlet, and an outlet that is downstream of the inlet, wherein the outlet has a conduit that is in fluid communication with the fluid flow path, and wherein the conduit of the outlet of the clot container has a minimum internal diameter of 2 mm or approximately 2 mm.

Embodiment 54: The vacuum aspiration system of Embodiment 54, comprising at least one conduit extending from a sealing element at a proximal end of the catheter sheath to the inlet of the clot container having an internal diameter that is 7 mm or approximately 7 mm.

Embodiment 55: The vacuum aspiration system of Embodiment 54, comprising at least one conduit extending from a sealing element at a proximal end of the catheter sheath to the inlet of the clot container having an internal diameter of from 6 mm or approximately 6 mm to 8 mm or approximately 8 mm.

Embodiment 56: The vacuum aspiration system of any one of the previous Embodiments, wherein dropping the flow rate of fluid through the fluid flow path to the second flow rate range after the first period of time reduces a loss of blood from a patient during a thrombectomy procedure.

Embodiment 57: The vacuum aspiration system of any one of the previous Embodiments, wherein the catheter sheath is a 24 Fr catheter sheath.

Embodiment 58: The vacuum aspiration system of any one of the previous Embodiments, wherein the catheter sheath is a 16 Fr catheter sheath.

Embodiment 59: The vacuum aspiration system of any one of the previous Embodiments, wherein the vacuum aspiration valve is configured to be moved between the open stated and the closed state by a robotic surgical system.

Embodiment 60: The vacuum aspiration system of any one of the previous Embodiments, comprising a robotic surgical system configured to at least move the aspiration control valve between the open stated and the closed state.

Embodiment 61: The vacuum aspiration system of any one of the previous Embodiments, wherein the aspiration catheter assembly has a constrictor downstream of a clot container configured to reduce the flow rate of the fluid through the fluid flow path downstream of the clot container.

Embodiment 62: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration valve, when the aspiration control valve is moved to an open state, the source of suction will provide the suction pressure through the fluid flow path upstream of the aspiration control valve such that:
      a flow rate of a fluid through the fluid flow path increases to a first flow rate range that is greater than zero;
      while the aspiration control valve continues to be in the open state, the flow rate of the fluid through the fluid flow path is maintained at the first flow rate range until a first volume of the fluid has been aspirated through the catheter sheath; and
      while the aspiration control valve continues to be in the open state, without any substantial change to the suction pressure provided by the source of suction, after the first volume of the fluid has been aspirated through the catheter sheath, the flow rate of the fluid through the fluid flow path drops to a second flow rate range that is greater than zero but less than the first flow rate range.

Embodiment 63: The vacuum aspiration system of Embodiment 62, wherein the first volume of the fluid is from 10 ml or approximately 10 ml to 30 ml or approximately 30 ml.

Embodiment 64: The vacuum aspiration system of any one of Embodiments 62-63, wherein the first volume of the fluid is from 15 ml or approximately 15 ml to 60 ml or approximately 60 ml.

Embodiment 65: The vacuum aspiration system of any one of Embodiments 62-64, wherein the first volume of the fluid is from 15 ml or approximately 15 ml to 25 ml or approximately 25 ml.

Embodiment 66: The vacuum aspiration system of any one of Embodiments 62-65, wherein the first flow rate range is greater than 100 ml per second.

Embodiment 67: The vacuum aspiration system of any one of Embodiments 62-66, wherein the first flow rate range is greater than 150 ml per second.

Embodiment 68: The vacuum aspiration system of any one of Embodiments 62-68, wherein the first flow rate range is at least 180 ml per second when the fluid being aspirated is water, the source of suction is a pump that provides at least a −28 inHg suction pressure, and the catheter sheath is a 24 Fr or larger catheter sheath.

Embodiment 69: The vacuum aspiration system of any one of Embodiments 62-68, wherein the first flow rate range is at least 160 ml per second when the fluid being aspirated is water, the source of suction is a 60 cc syringe, and the catheter sheath is a 24 Fr or larger catheter sheath.

Embodiment 70: The vacuum aspiration system of any one of Embodiments 62-68, wherein the first flow rate range is at least 80 ml per second when the fluid being aspirated is water, the source of suction is a pump that provides at least a −28 inHg suction pressure, and the catheter sheath is a 16 Fr or larger catheter sheath.

Embodiment 71: The vacuum aspiration system of any one of Embodiments 62-70, wherein the first flow rate range is at least 60 ml per second when the fluid being aspirated is water, the source of suction is a 60 cc syringe, and the catheter sheath is a 16 Fr or larger catheter sheath.

Embodiment 72: The vacuum aspiration system of any one of Embodiments 62-71, wherein a substantial change would be more than a 10% change in the suction pressure provided by the source of suction.

Embodiment 73: The vacuum aspiration system of any one of Embodiments 62-72, wherein the second flow rate range is less than half of the first flow rate range.

Embodiment 74: The vacuum aspiration system of any one of Embodiments 62-73, wherein the second flow rate range is less than 15% of the first flow rate range.

Embodiment 75: The vacuum aspiration system of any one of Embodiments 62-74, wherein the second flow rate range is less than 20 ml per second.

Embodiment 76: The vacuum aspiration system of any one of Embodiments 62-75, wherein the second flow rate range is less than 10 ml per second.

Embodiment 77: The vacuum aspiration system of any one of Embodiments 62-76, comprising the source of suction.

Embodiment 78: The vacuum aspiration system of any one of Embodiments 62-77, wherein the source of suction applies a suction pressure of at least −28 inHg or at least approximately −28 inHg.

Embodiment 79: The vacuum aspiration system of any one of Embodiments 62-78, wherein the source of suction is a vacuum pump.

Embodiment 80: The vacuum aspiration system of any one of Embodiments 62-79, wherein the source of suction is a 60 cc syringe.

Embodiment 81: The vacuum aspiration system of any one of Embodiments 62-80, further comprising a suction conduit in communication with the fluid flow path and configured to be fluidically couplable with the source of suction.

Embodiment 82: The vacuum aspiration system of any one of Embodiments 62-81, wherein the aspiration control valve is moved from the closed state to the open state rapidly so that the suction pressure is provided rapidly to the fluid flow path upstream of the aspiration control valve.

Embodiment 83: The vacuum aspiration system of any one of Embodiments 62-82, wherein the vacuum aspiration system is configured to provide a rapid burst of suction through the catheter when the aspiration control valve is moved to the open state so that the catheter reaches a flow rate of at least 190 ml per second in less than 0.09 seconds, when the fluid is water, the source of suction is a pump that provides at least a −28 inHg suction pressure, and the catheter sheath is a 24 Fr or larger catheter sheath.

Embodiment 84: The vacuum aspiration system of any one of Embodiments 62-83, wherein the vacuum aspiration system is configured to provide a rapid burst of suction through the catheter when the aspiration control valve is moved to the open state so that the catheter reaches a flow rate of at least 70 ml per second in less than 0.09 seconds, when the fluid is water, the source of suction is a pump that provides at least a −28 inHg suction pressure, and the catheter sheath is a 16 Fr or larger catheter sheath.

Embodiment 85: The vacuum aspiration system of any one of Embodiments 62-84, wherein the vacuum aspiration system is configured to provide a rapid burst of suction through the catheter when the aspiration control valve is moved to the open state so that the catheter reaches a flow rate of at least 160 ml per second in less than 0.09 seconds, when the fluid is water, the source of suction is a 60 cc syringe, and the catheter sheath is a 24 Fr or larger catheter sheath.

Embodiment 86: The vacuum aspiration system of any one of Embodiments 62-85, wherein the vacuum aspiration system is configured to provide a rapid burst of suction through the catheter when the aspiration control valve is moved to the open state so that the catheter reaches a flow rate of at least 60 ml per second in less than 0.08 seconds, when the fluid is water, the source of suction is a 60 cc syringe, and the catheter sheath is a 16 Fr or larger catheter sheath.

Embodiment 87: The vacuum aspiration system of any one of Embodiments 62-86, wherein the vacuum aspiration system is configured to provide a rapid burst of suction through the catheter when the aspiration control valve is moved to the open state so that the catheter reaches a flow rate of at least 160 ml per second in less than 0.12 seconds, when the fluid is a blood analog, the source of suction is a pump that provides at least a −28 inHg suction pressure, and the catheter sheath is a 24 Fr or larger catheter sheath.

Embodiment 88: The vacuum aspiration system of any one of Embodiments 62-87, wherein the vacuum aspiration system is configured to provide a rapid burst of suction through the catheter when the aspiration control valve is moved to the open state so that the catheter reaches a flow rate of at least 50 ml per second in less than 0.13 seconds, when the fluid is a blood analog, the source of suction is a pump that provides at least a −28 inHg suction pressure, and the catheter sheath is a 16 Fr or larger catheter sheath.

Embodiment 89: The vacuum aspiration system of any one of Embodiments 62-88, wherein a portion of the fluid flow path of the catheter upstream of the aspiration control valve is primed with the fluid before the aspiration control valve is moved to the open state.

Embodiment 90: The vacuum aspiration system of any one of Embodiments 62-89, wherein the fluid is blood.

Embodiment 91: The vacuum aspiration system of any one of Embodiments 62-90, wherein the fluid is a blood analog.

Embodiment 92: The vacuum aspiration system of any one of Embodiments 62-91, wherein the fluid is water.

Embodiment 93: The vacuum aspiration system of Embodiment 92, wherein the fluid is water, and a peak flow rate of the water through the fluid flow path is at least 183 ml per second.

Embodiment 94: The vacuum aspiration system of any one of Embodiments 62-93, wherein the aspiration catheter assembly is configured to provide an average flow rate of at least 120 ml per second during a period of time from when the aspiration control valve is moved to the open state to when the flow rate drops to the second flow rate range, when the fluid is water, the source of suction is a pump that provides at least a −28 inHg suction pressure, and the catheter sheath is a 24 Fr or larger catheter sheath.

Embodiment 95: The vacuum aspiration system of any one of Embodiments 62-94, wherein the aspiration catheter assembly is configured to provide an average flow rate of at least 65 ml per second during a period of time from when the aspiration control valve is moved to the open state to when the flow rate drops to the second flow rate range, when the fluid is water, the source of suction is a pump that provides at least a −28 inHg suction pressure, and the catheter sheath is a 16 Fr or larger catheter sheath.

Embodiment 96: The vacuum aspiration system of any one of Embodiments 62-95, wherein the vacuum aspiration system is configured so that the first flow rate range of the fluid through the catheter is sustained for no more than 0.2 seconds.

Embodiment 97: The vacuum aspiration system of any one of Embodiments 62-96, wherein the vacuum aspiration system is configured so that the first flow rate range of the fluid through the catheter is sustained for no more than 0.25 seconds.

Embodiment 98: The vacuum aspiration system of any one of Embodiments 62-97, wherein the vacuum aspiration system is configured so that the first flow rate range of the fluid through the catheter is sustained for no more than 0.15 seconds.

Embodiment 99: The vacuum aspiration system of any one of Embodiments 62-98, wherein the vacuum aspiration system is configured to provide a rapid drop-off of the suction provided through the catheter such that the flow rate of the fluid through the fluid flow path automatically drops below 20 mL per second within 0.25 seconds after the aspiration control valve is moved to the open state, with the aspiration control valve remaining in an open state, when the fluid is water, blood, or a blood analog.

Embodiment 100: The vacuum aspiration system of any one of Embodiments 62-99, wherein the vacuum aspiration system is configured to provide a rapid drop-off of the suction provided through the catheter such that the flow rate of the fluid through the fluid flow path automatically drops below 30 mL per second within 0.5 seconds after the aspiration control valve is moved to the open state, with the aspiration control valve remaining in an open state, when the fluid is water, blood, or a blood analog and the source of suction is a suction pump or a syringe.

Embodiment 101: The vacuum aspiration system of any one of Embodiments 62-100, wherein the vacuum aspiration system is configured to provide a rapid drop-off of the suction provided through the catheter such that the flow rate of the fluid through the fluid flow path drops below 10 mL per second within 0.3 seconds after the flow rate of the fluid through the fluid flow path first reaches the peak flow rate while the aspiration control valve remains in an open state.

Embodiment 102: The vacuum aspiration system of any one of Embodiments 62-101, wherein the aspiration control valve comprises a lever.

Embodiment 103: The vacuum aspiration system of any one of Embodiments 62-102, further comprising a clot container coupled with the housing, the clot container being in fluid communication with the fluid flow path.

Embodiment 104: The vacuum aspiration system of Embodiment 103, wherein the clot container has a filter therein.

Embodiment 105: The vacuum aspiration system of Embodiment 103, wherein the clot container has a volume of 17.7 ml or approximately 17.7 ml.

Embodiment 106: The vacuum aspiration system of Embodiment 103, wherein the clot container has a volume of at least 17 ml or at least approximately 17.7 ml.

Embodiment 107: The vacuum aspiration system of Embodiment 103, wherein the clot container has a volume of at least 40 ml or at least approximately 40 ml.

Embodiment 108: The vacuum aspiration system of Embodiment 103, wherein the clot container has a volume of at least 60 ml or at least approximately 60 ml.

Embodiment 109: The vacuum aspiration system of Embodiment 103, wherein the first period of time is increased by increasing the volume of the clot container and/or a volume of the fluid flow path upstream of the clot container.

Embodiment 110: The vacuum aspiration system of any one of Embodiments 62-109, wherein the fluid flow path passes through a suction conduit downstream of aspiration control valve, wherein the suction conduit is in fluidic communication with the source of suction and wherein the suction conduit has an internal diameter of from 2.5 mm or approximately 2.5 mm to 2.8 mm or approximately 2.8 mm.

Embodiment 111: The vacuum aspiration system of any one of Embodiments 62-110, wherein the fluid flow path passes through at least two tubes comprising the suction conduit downstream of aspiration control valve, wherein the at least two tubes are in fluidic communication with the source of suction, wherein an internal diameter of each of the at least two tubes is from 2.5 mm or approximately 2.5 mm to 2.8 mm or approximately 2.8 mm, and wherein a combined length of the at least two tubes is more than 100 inches.

Embodiment 112: The vacuum aspiration system of any one of Embodiments 62-111, further comprising a clot container coupled with the housing, the clot container having an internal space, an inlet, and an outlet that is downstream of the inlet, wherein the outlet has a conduit that is in fluid communication with the fluid flow path, and wherein the conduit of the outlet of the clot container has a minimum internal diameter of 2 mm or approximately 2 mm.

Embodiment 113: The vacuum aspiration system of Embodiment 112, comprising at least one conduit extending from a sealing element at a proximal end of the catheter sheath to the inlet of the clot container having an internal diameter that is 7 mm or approximately 7 mm.

Embodiment 114: The vacuum aspiration system of Embodiment 112, comprising at least one conduit extending from a sealing element at a proximal end of the catheter sheath to the inlet of the clot container having an internal diameter of from 6 mm or approximately 6 mm to 8 mm or approximately 8 mm.

Embodiment 115: The vacuum aspiration system of any one of Embodiments 62-114, wherein dropping the flow rate of fluid through the fluid flow path to the second flow rate range after the first period of time reduces a loss of blood from a patient during a thrombectomy procedure.

Embodiment 116: The vacuum aspiration system of any one of Embodiments 62-115, wherein the catheter sheath is a 24 Fr catheter sheath.

Embodiment 117: The vacuum aspiration system of any one of Embodiments 62-116, wherein the catheter sheath is a 16 Fr catheter sheath.

Embodiment 118: The vacuum aspiration system of any one of Embodiments 62-117, wherein the vacuum aspiration valve is configured to be moved between the open stated and the closed state by a robotic surgical system.

Embodiment 119: The vacuum aspiration system of any one of Embodiments 62-118, comprising a robotic surgical system configured to at least move the aspiration control valve between the open stated and the closed state.

Embodiment 120: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration control valve, when the aspiration control valve is moved to an open state, the source of suction will provide the suction pressure through the fluid flow path upstream of the aspiration control valve such that a peak flow rate of a fluid through the fluid flow path is achieved within 0.1 seconds after the aspiration control valve is moved to the open state.

Embodiment 121: The vacuum aspiration system of Embodiment 120, wherein the peak flow rate is between at least 190 ml per second and 230 ml per second, when the fluid is water, the source of suction is a pump that provides at least a −28 inHg suction pressure, and the catheter sheath is a 24 Fr or larger catheter sheath.

Embodiment 122: The vacuum aspiration system of any one of Embodiments 120-, wherein the peak flow rate is between at least 70 ml per second and 100 ml per second, when the fluid is water, the source of suction is a pump that provides at least a −28 inHg suction pressure, and the catheter sheath is a 16 Fr or larger catheter sheath.

Embodiment 123: The vacuum aspiration system of any one of Embodiments 120-122, wherein the peak flow rate is between at least 160 ml per second and 190 ml per second, when the fluid is water, the source of suction is a syringe, and the catheter sheath is a 24 Fr or larger catheter sheath.

Embodiment 124: The vacuum aspiration system of any one of Embodiments 120-123, wherein the peak flow rate is between at least 60 ml per second and 80 ml per second, when the fluid is water, the source of suction is a syringe, and the catheter sheath is a 16 Fr or larger catheter sheath.

Embodiment 125: The vacuum aspiration system of any one of Embodiments 120-124, wherein the peak flow rate is between at least 160 ml per second and 230 ml per second, when the fluid is a blood analog, the source of suction is a pump that provides at least a −28 inHg suction pressure, and the catheter sheath is a 24 Fr or larger catheter sheath.

Embodiment 126: The vacuum aspiration system of any one of Embodiments 120-125, wherein the vacuum aspiration valve is configured to be moved between the open stated and the closed state by a robotic surgical system.

Embodiment 127: The vacuum aspiration system of any one of Embodiments 120-126, comprising a robotic surgical system configured to at least move the aspiration control valve between the open stated and the closed state.

Embodiment 128: A vacuum aspiration system comprising:
an aspiration catheter assembly comprising:
   a catheter sheath extending from a proximal end to a distal end;
   a housing at the proximal end of the catheter sheath;
   a fluid flow path extending through the housing and the catheter sheath and configured to selectively receive a suction pressure from a source of suction; and
   an aspiration control valve in fluid communication with the fluid flow path, the aspiration control valve configured to control the suction pressure through the fluid flow path from the source of suction upstream of the aspiration control valve;
wherein:
   the vacuum aspiration system is configured such that, with the aspiration control valve in a closed state and the source of suction providing the suction pressure to the fluid flow path up to the aspiration control valve, when the aspiration control valve is moved to an open state, the source of suction will provide the suction pressure through the fluid flow path upstream of the aspiration control valve such that a peak flow rate of a fluid through the fluid flow path is achieved within 0.15 seconds after the aspiration control valve is moved to the open state.

Embodiment 129: The vacuum aspiration system of Embodiment 128, wherein the peak flow rate is between at least 160 ml per second and 200 ml per second, when the fluid is a blood analog, the source of suction is a pump that provides at least a −28 inHg suction pressure, and the catheter sheath is a 24 Fr or larger catheter sheath.

Embodiment 130: The vacuum aspiration system of any one of Embodiments 128-129, wherein the peak flow rate is between at least 75 ml per second and 95 ml per second, when the fluid is a blood analog, the source of suction is a pump that provides at least a −28 inHg suction pressure, and the catheter sheath is a 16 Fr or larger catheter sheath.

Embodiment 131: A method of aspirating a fluid through a catheter of an aspiration system, comprising:
positioning an aspiration control valve of the catheter in a closed position;
with the aspiration control valve in the closed position and a fluid flow path of the catheter in fluid communication with the fluid, applying a suction pressure to the fluid flow path of the catheter from a source of suction;
moving the aspiration control valve to an open position to aspirate the fluid through the fluid flow path of the catheter, wherein a flow rate of the fluid through the fluid flow path increases to a first flow rate range that is greater than 50 ml per second; and
when the flow rate of the fluid through the fluid flow path decreases below a second value, moving the aspiration control valve back to the closed position;
wherein:
   the catheter is configured to decrease the flow rate of the fluid through the fluid flow path without changing the suction pressure being applied to the fluid flow path of the catheter by the source of suction.

Embodiment 132: The method of Embodiment 131, wherein the catheter is configured to decrease the flow rate of the fluid through the fluid flow path to the second value of the flow rate range without making any changes to the aspiration system.

Embodiment 133: The method of any one of Embodiments 130-132, wherein the first flow rate range is greater than 100 ml per second or approximately 100 ml per second, or is greater than 150 ml per second or approximately 150 ml per second, or is greater than 180 ml per second or approximately 180 ml per second, or is greater than 190 ml per second or approximately 190 ml per second, or is greater than 200 ml per second or approximately 200 ml per second, or is from 100 ml per second or is approximately 100 ml per second to 200 ml per second or approximately 200 ml per second, or is from 150 ml per second or approximately 150 ml per second to 200 ml per second or approximately 200 ml per second, or of any value, approximate value, or range of values in any of the foregoing ranges.

Embodiment 134: The method of any one of Embodiments 130-133, wherein the second value is between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges.

Embodiment 135: A method of aspirating a blood clot through a catheter of an aspiration system, comprising:
positioning an aspiration control valve of the catheter in a closed position;
with the aspiration control valve in the closed position and a fluid flow path of the catheter in fluid communication with the fluid, applying a suction pressure to the fluid flow path of the catheter from a source of suction;
moving the aspiration control valve to an open position to aspirate blood through the fluid flow path of the catheter, wherein a flow rate of the blood through the fluid flow path increases to a first flow rate range that is greater than 50 ml per second; and
when the blood begins flowing into a clot container of the catheter, moving the aspiration control valve back to the closed position;
wherein:
   the catheter is configured to automatically decrease the flow rate of the fluid through the fluid flow path to less than 15 ml per second without any change to the suction pressure being applied to the fluid flow path of the catheter by the source of suction and without moving the aspiration control valve to the closed position.

Embodiment 136: A method of aspirating a clot material during a thrombectomy procedure, comprising:
positioning an aspiration control valve of an aspiration catheter in a closed position;
applying a suction pressure to a fluid flow path of the catheter;
positioning an aspiration catheter within a predetermined distance of a clot within a patient's vasculature;
moving the aspiration control valve to an open position to aspirate the clot through the fluid flow path of the aspiration catheter, wherein a flow rate of a fluid through the fluid flow path upstream of the aspiration control valve increases to a first flow rate range that is greater than zero;
when the flow rate of the fluid through the fluid flow path decreases below a second flow rate value, moving the aspiration control valve back to the closed position;
after the flow rate of the fluid through the fluid flow path has decreased below the second flow rate value and after moving the aspiration control valve back to the closed position, withdrawing the aspiration catheter a predetermined distance; and
after withdrawing the aspiration catheter the predetermined distance, moving the aspiration control valve again to the open position to continue to aspirate the clot through the fluid flow path of the aspiration catheter, wherein the flow rate of the fluid through the fluid flow path upstream of the aspiration control valve increases again to the first flow rate range.

Embodiment 137: The method of Embodiment 136, further comprising, when the flow rate of the fluid through the fluid flow path again decreases below the second flow rate value, moving the aspiration control valve back to the closed position.

Embodiment 138: The method of Embodiment 137, further comprising,
withdrawing the aspiration catheter a second predetermined distance; and
after withdrawing the aspiration catheter the second predetermined distance, moving the aspiration control valve again to the open position to continue to aspirate the clot through the fluid flow path of the aspiration catheter, wherein the flow rate of the fluid through the fluid flow path upstream of the aspiration control valve increases again to the first flow rate range.

Embodiment 139: The method of Embodiment 138, further comprising, when the flow rate of the fluid through the fluid flow path decreases below the second flow rate value for a third time, moving the aspiration control valve back to the closed position.

Embodiment 140: The method of any one of Embodiments 136-139, wherein the first flow rate range is greater than 40 ml per second, when the fluid is blood.

Embodiment 141: The method of any one of Embodiments 136-140, wherein the first flow rate range is greater than 50 ml per second, when the fluid is blood.

Embodiment 142: The method of any one of Embodiments 136-141, wherein the first flow rate range is greater than 60 ml per second, when the fluid is blood.

Embodiment 143: The method of any one of Embodiments 136-142, wherein the first flow rate range is from 40 ml per second to 120 ml per second, when the fluid is blood.

Embodiment 144: The method of any one of Embodiments 136-143, wherein the second flow rate value is from 50 ml per second to 100 ml per second, when the fluid is blood.

Embodiment 145: The method of any one of Embodiments 136-144, wherein the second flow rate value is between 2 ml per second or approximately 2 ml per second and 20 ml per second or approximately 20 ml per second, or between 5 ml per second or approximately 5 ml per second and 10 ml per second or approximately 10 ml per second, or of any value, approximate value, or range of values in any of the foregoing ranges.

Embodiment 146: The method of any one of Embodiments 136-145, wherein the fluid is blood.

Embodiment 147: The method of any one of Embodiments 136-146, wherein the flow rate of the fluid through the fluid flow path decreases below the second flow rate value when a clot container of the aspiration catheter becomes full with the fluid or the fluid and the clot material.

Embodiment 148: The method of any one of Embodiments 136-147, wherein the aspiration catheter is configured to decrease the flow rate of the fluid through the fluid flow path below the second flow rate value without any user input.

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 30, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of aspirating a substance with an aspiration device during an aspiration procedure, comprising:
with an aspiration control valve of the aspiration device in a closed position, applying a suction pressure to a fluid flow path of the aspiration device;
positioning a catheter of the aspiration device within a patient's vasculature;
moving the aspiration control valve to an open position to aspirate the substance through at least the catheter of the aspiration device so that a flow rate of the substance through the catheter increases to at least a first flow rate range that is greater than zero;
when the flow rate of the substance through the catheter decreases below a second flow rate value that is lower than the first flow rate range, moving the aspiration control valve to the closed position;
moving the catheter in a proximal direction or in a distal direction;
after moving the catheter in the proximal direction or the distal direction, moving the aspiration control valve again to the open position to continue to aspirate the substance through at least the catheter.

2. The method of claim 1, wherein moving the aspiration control valve again to the open position to continue to aspirate the substance through at least the catheter comprises aspirating the substance through at least the catheter at a rate that is at least as great as the first flow rate range.

3. The method of claim 1, wherein moving the aspiration catheter in the proximal direction or the distal direction comprises moving the aspiration catheter in the proximal direction or the distal direction by a distance that is 2 cm or approximately 2 cm.

4. The method of claim 1, wherein moving the aspiration catheter in the proximal direction or the distal direction comprises moving the aspiration catheter in the proximal direction or the distal direction by a distance that is from 1 cm or approximately 1 cm to 3 cm or approximately 3 cm.

5. The method of claim 1, wherein moving the aspiration catheter in the proximal direction or the distal direction comprises moving the aspiration catheter in the proximal direction or the distal direction by a distance that is no greater than 3 cm.

6. The method of claim 1, further comprising, when the flow rate of the substance through the fluid flow path again decreases below the second flow rate value, moving the aspiration control valve back to the closed position.

7. The method of claim 6, further comprising:
after moving the aspiration control valve back to the closed position, moving the aspiration catheter again in the proximal direction or the distal direction; and
after again moving the aspiration catheter in the proximal direction or the distal direction, again moving the aspiration control valve to the open position to continue to aspirate the substance through the fluid flow path of the aspiration catheter.

8. The method of claim 7, wherein again moving the aspiration catheter in the proximal direction or the distal direction comprises again moving the aspiration catheter by a distance that is 2 cm or approximately 2 cm.

9. The method of claim 7, wherein again moving the aspiration catheter in the proximal direction or the distal direction comprises again moving the aspiration catheter by a distance that is from 1 cm or approximately 1 cm to 3 cm or approximately 3 cm.

10. The method of claim 7, further comprising, when the flow rate of the substance through the fluid flow path again decreases below the second flow rate value, moving the aspiration control valve back to the closed position.

11. The method of claim 1, wherein the first flow rate range is greater than 60 ml per second.

12. The method of claim 1, wherein the lowermost value of the first flow rate range is greater than 120 ml per second.

13. The method of claim 1, wherein the aspiration catheter is configured to decrease the flow rate of the substance through the fluid flow path below the second flow rate value while the aspiration control valve is in the open position without any user input or any change to the suction pressure provided to the aspiration device by a source of suction.

14. The method of claim 1, wherein the second flow rate range is greater than zero and is less than 50% of the first flow rate range.

15. A method of aspirating a substance from a patient's vasculature during an aspiration procedure, comprising:
providing an aspiration system comprising:
a catheter in communication with an aspiration pump through an aspiration line;
a chamber in the aspiration line closer to the catheter than the pump; and
a valve in the aspiration line between the catheter and the chamber;
activating the aspiration pump to apply a suction force to the chamber;
positioning the catheter within aspiration range of a clot;
opening the valve a first time to aspirate the substance toward the chamber;
closing the valve;
moving the catheter in a distal or a proximal direction; and
opening the valve a second time to aspirate the substance toward the chamber.

16. The method of claim 15, comprising opening a vent to evacuate the chamber after closing the valve.

17. The method of claim 15, wherein, when the valve is an open state, the aspiration pump aspirates the substance through at least the chamber to fill the chamber.

18. The method of claim 15, wherein moving the catheter comprises moving the catheter a distance that is from 1 to 3 cm.
